(19) United States
(12) Patent Application Publication
(10) Pub. No.: US 2024/0042067 A1
(43) Pub. Date: Feb. 8, 2024

(54) [177LU] LUTETIUM-PSMA I&T COMPOSITION, KIT, METHOD OF MAKING, AND METHOD OF USING THEREOF

(71) Applicant: Curium US LLC, St. Louis, MO (US)

(72) Inventors: Jarno Jalomäki, Helsinki (FI); Salla Seppänen, Helsinki (FI); Amanda Donovan, St. Louis, MO (US); Jessica Salem, St. Louis, MO (US)

(21) Appl. No.: 18/228,510

(22) Filed: Jul. 31, 2023

Related U.S. Application Data
(60) Provisional application No. 63/393,446, filed on Jul. 29, 2022.
(60) Provisional application No. 63/393,777, filed on Jul. 29, 2022.

(51) Int. Cl. A61K 51/08; A61K 9/00; A61K 47/22; A61K 47/18; A61K 47/10; A61P 35/04; A61B 6/00

(52) U.S. Cl. CPC A61K 51/088; A61K 9/0019; A61K 47/22; A61K 47/183; A61K 47/10; A61P 35/04; A61B 6/4057; A61J 1/065

(57) ABSTRACT

The present disclosure generally relates to a radiopharmaceutical composition comprising ¹⁷⁷Lu-PSMA I&T. The composition is formulated as a solution for injection and the solution is suitable for administration more than 72 hours after formulation. The composition may include ascorbic acid and have a pH of 3.5 to 4.5 in solution. In some examples, the composition is suitable for administration to a human patient in need thereof at least 90 hours after formulation, and the composition has a radiochemical purity of 95% or greater at administration.

### RELATED APPLICATIONS

This application claims the benefit under 35 U.S.C. § 119 of U.S. Application No. 63/393,777 filed Jul. 29, 2022, and U.S. Application No. 63/393,446 filed Jul. 29, 2022, the entire contents of each of which are incorporated herein by reference.

### FIELD

The present disclosure generally relates to a new composition of a [¹⁷⁷Lu]Lutetium-PSMA I&T ([¹⁷⁷Lu]Lu-PSMA I&T or ¹⁷⁷Lu-PSMA I&T) solution for injection, as well as a kit comprising ¹⁷⁷Lu-PSMA I&T. The ¹⁷⁷Lu-PSMA I&T solution and/or kit thereof may be used for prostate cancer radioligand therapy (PRLT).

### BACKGROUND

Prostate cancer (PC) is the most frequent non-cutaneous cancer and the second most frequent cause of cancer deaths for adult men. In 2020, there were more than 1.4 million new cases of PC, and 375,304 deaths worldwide. Most deaths related to prostate cancer are due to advanced disease, which results from any combination of lymphatic, blood, or contiguous local spread. Most patients with PC who die, die of metastatic PC, and 90% of these patients present with bone metastases. In some instances, surgery and/or chemotherapy may not be available or effective for a particular patient, including patients with metastatic castrate resistant prostate cancer (mCRPC). Therefore, alternatives are needed in the treatment of prostate cancer.

During the last decade, six new drugs have been found to increase overall survival for patients with metastatic castration-resistant prostate cancer (mCRPC). Patients with symptomatic mCRPC have initially been treated with docetaxel. Abiraterone, enzalutamide, cabazitaxel, sipuleucel, and radium-223 increase overall survival for patients who had failed treatment with docetaxel. Targeted radionuclide therapy has become an attractive and quickly developing therapy option for many different cancers, such as lymphoma, melanoma, and neuroendocrine tumors.

However, randomized trials have not evaluated the drugs for patients with failure in response to second-line treatment following recurrence after docetaxel. Therefore, international organizations such as European Association of Urology (EAU)/European Society of Radiotherapy and Oncology (ESTRO) have guidelines, but no recommendations, for third-line treatment of mCRPC. There exists a clinical need for an effective third line treatment for mCRPC with lower rates of toxicity. [¹⁷⁷Lu]Lu-PSMA-617 is a new and promising therapy option for patients with mCRPC.

Prostate-specific membrane antigen (PSMA) is highly expressed on prostate epithelial cells and strongly up-regulated in prostate cancer. The PSMA expression levels are directly correlated to androgen independence, metastasis, and prostate cancer progression. Thus, PSMA is a promising molecular target for diagnosis and therapy of metastatic prostate cancer.

Some monoclonal antibodies (mAbs) radiolabeled with Lutetium-177 (¹⁷⁷Lu) or Yttrium-90 (⁹⁰Y), showed promising results; however, there were higher rates of hematological toxicity. Due to the side effects, it is therefore prudent to consider small molecule inhibitors of PSMA instead of mAbs. ¹⁷⁷Lu-PSMA-617 and ¹⁷⁷Lu-PSMA I&T are small-molecule inhibitors of PSMA that are extremely desirable for targeted radionuclide therapy due to their low hematotoxicity and nephrotoxicity profiles, providing better effects and fewer adverse effects.

¹⁷⁷Lu is α 3- and γ-emitting radionuclide which can irradiate tumor cells. ¹⁷⁷Lu has a half-life of 6.7 days and a lower beta-particle emission energy than Iodine-131 (¹³¹I), indicating higher probability for less side effects. Compositions made with ¹⁷⁷Lu generally have a short shelf life. Previous imaging and therapy formulations with ¹⁷⁷Lu-PSMA have a shelf life of about 48 hours. This necessitates synthesis of the compounds and final solution within close proximity of where it will be administered and are generally prepared to order. Because of limited resources, this could limit use of a vital cancer treatment to a limited number of geographical locations.

There continues to be a need for an improved formulation containing ¹⁷⁷Lu-PSMA I&T with a shelf life of more than 48 hours. The present disclosure is directed to an improved ¹⁷⁷Lu-PSMA I&T solution for injection.

### SUMMARY

Briefly, therefore, the present disclosure is directed to a radiopharmaceutical composition comprising ¹⁷⁷Lu-PSMA I&T for administration to a human patient in need thereof. The composition is formulated as a solution for injection and the solution is suitable for administration to a human patient in need thereof more than 48 hours, more than 72 hours, more than 96 hours, or more than 100 hours after formulation.

The present disclosure is further directed to a radiopharmaceutical composition comprising ¹⁷⁷Lu-PSMA I&T and ascorbic acid at a pH of 3.5 to 4.5 in solution. In some examples, the composition is suitable for administration to a human patient in need thereof at least 90 hours after formulation, and the composition has a radiochemical purity of 95.0% or greater at administration.

Another aspect of the present disclosure is a reaction composition comprising ¹⁷⁷Lu, about 463 to 500 μg/mL PSMA I&T precursor in an ascorbic acid and acetate buffer. Another aspect of the present disclosure is a reaction composition comprising ¹⁷⁷Lu, about 463 to 500 μg/mL PSMA I&T precursor, about 4 ml 0.4 M sodium acetate, about 1.6 mL 0.05 M hydrochloric acid, and about 150 μl 20% L-ascorbic acid in a total of 6 to 8 mL of solution. In yet another aspect of the present disclosure is a reaction composition comprising ¹⁷⁷Lu, about 463 to 1000 μg/mL PSMA I&T precursor in an ascorbic acid and acetate buffer. In some examples, the reaction composition has a ¹⁷⁷Lu radioactivity ≤61 GBq.

Another aspect of the present disclosure is a reaction composition comprising ¹⁷⁷Lu, PSMA I&T precursor, ascorbic acid/acetate buffer, hydrochloric acid, and L-ascorbic acid in a solution. In some examples, the reaction composition has a ¹⁷⁷Lu radioactivity ≤296 GBq.

The present disclosure is further directed to a radiopharmaceutical composition comprising ¹⁷⁷Lu-PSMA I&T solution for injection comprising ¹⁷⁷Lu-PSMA I&T, ascorbic acid, and ethanol. wherein the ¹⁷⁷Lu-PSMA I&T is in sufficient amounts of radioactivity for intended use, wherein the total amount of ascorbic acid in the solution is about 210-700 mg and the total amount of ethanol in the solution is about 274-706 mg; wherein pH of the solution is about 5 or below, wherein upon administration of the composition to a subject, the subject maintains low levels of hematotoxic and nephrotoxic toxicity, and wherein the prostate-specific antigen decline is more than about 50%.

The present disclosure is further directed to a radiopharmaceutical composition comprising ¹⁷⁷Lu-PSMA I&T solution for injection comprising ¹⁷⁷Lu-PSMA-I&T in an amount from about 5 μg/ml to about 15 μg/ml, ascorbic acid in a concentration from about 10 mg/ml to about 50 mg/ml, and ethanol in a concentration of about 1% (v/v) to about 10% (v/v), wherein pH of the solution is between about 3 and about 5, and wherein upon administration of the composition to a subject, the subject maintains low levels of hematotoxic and nephrotoxic toxicity.

The present disclosure is further directed to a radiopharmaceutical composition comprising ¹⁷⁷Lu-PSMA I&T solution for injection comprising ¹⁷⁷Lu-PSMA I&T in an amount from about 5 μg/ml to about 15 μg/ml, ascorbic acid in a concentration from about 10 mg/ml to about 50 mg/ml, ethanol in a concentration of about 1% (v/v) to about 10% (v/v), and a chelating agent in an amount from about 0.001% to about 0.15% (w/w) of the total weight of the radiopharmaceutical composition, wherein pH of the solution is between about 3 and about 5, and wherein upon administration of the composition to a subject, the subject maintains low levels of hematotoxic and nephrotoxic toxicity.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.1 GBq dose.

The present disclosure is further related to a radiopharmaceutical kit, comprising a vial containing at least a single dose of a ¹⁷⁷Lu-PSMA I&T solution for injection to a human patient in need thereof, wherein the injection comprises a dose of 177Lu-PSMA-I&T, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy. The present disclosure is also related to a radiopharmaceutical kit, comprising a vial containing at least a single dose of a ¹⁷⁷Lu-PSMA I&T solution for injection to a human patient in need thereof, wherein the injection comprises a dose of 177Lu-PSMA-I&T, and the projected or actual cumulated kidney absorbed dose at 6 cycles is less than 23 Gy and no renal toxicities are observed. The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed. The present disclosure is further related to a radiopharmaceutical kit, comprising a vial containing at least a single dose of a ¹⁷⁷Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed. The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq (mean 7.52±0.16 GBq) dose of 177Lu-PSMA-I&T, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed. The present disclosure is also further related to a radiopharmaceutical kit, comprising a vial containing at least a single dose of a ¹⁷⁷Lu-PSMA I&T solution for injection to a human patient in need thereof, wherein the injection comprises a 7.4 GBq (mean 7.52±0.16 GBq) dose of 177Lu-PSMA-I&T, wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed. The present disclosure is also further related to a radiopharmaceutical kit, comprising a vial containing at least a single dose of a ¹⁷⁷Lu-PSMA I&T solution for injection to a human patient in need thereof, wherein the injection comprises a 7.4 (+/−10%) GBq dose of 177Lu-PSMA-I&T, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed. Note: the term “cumulative” may be substituted for “cumulated.”

The present disclosure is further related to a method of administering a radiopharmaceutical composition, the method comprising administering the radiopharmaceutical composition into a human patient in need thereof, optionally administering more than 48 hours after formulation, the radiopharmaceutical composition comprising a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose of ¹⁷⁷Lu-PSMA I&T in a solution comprising a pH of 3.5 to 4.5, the solution optionally comprising ascorbic acid and/or ethanol, and the solution optionally comprising a radiochemical purity of more than 95%, more than 96% more than 97%, more than 98%, more than 99%, or more than 99.5% when administered, and wherein 177Lu-PSMA I&T treatment with 6 cycles at the dose is possible without the risk of kidney toxicities and/or wherein 177Lu-PSMA I&T treatment with 6 cycles provides a mean projected dose that is below an absorbed dose limit of 23 Gy and/or the projected or actual cumulated kidney absorbed dose at 6 cycles is less than 23 Gy and no renal toxicities are observed.

The present disclosure is further related to a method of diagnosing or treating a tumor of a patient in need thereof, the method comprising administering by injection a radiopharmaceutical composition comprising ¹⁷⁷Lu-PSMA I&T and ascorbic acid at a pH of 3.5 to 4.5 in solution, wherein 20 hours after injection the radiopharmaceutical composition has an activity of at least 20% IA to 30% IA in the whole body.

The present disclosure is further related to a method of diagnosing a tumor of a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 8% IA to 10% IA in the kidneys.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.7% IA to 1% IA in the parotid glands.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.2% IA to 0.5% IA in lymph node lesions of the patient.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.1% IA to 0.4% IA in bone lesions of the patient.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein the radiopharmaceutical composition has an effective half-life of about 30 hours to 40 hours in the whole body of the patient.

Various refinements exist of the features noted above in relation to the various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the brief summary presented above is intended only to familiarize the reader with certain aspects and contexts of the present disclosure without limitation to the claimed subject matter.

### BRIEF DESCRIPTION OF THE FIGURES

Various features, aspects, and advantages of the present disclosure will become better understood when the following detailed description is read with reference to the accompanying figures in which like characters represent like parts throughout the figures, wherein:

FIG. 1A presents the structural formula of the precursor PSMA I&T.

FIG. 1B presents the structural formula for the R isomer of ¹⁷⁷Lu-PSMA I&T. However, the PSMA I&T doesn't have a specification for enantiomeric purity of R and S isomers.

FIG. 2 is a flowchart representation of an example method of preparing the disclosed radiopharmaceutical composition.

FIG. 3A is a flowchart of the synthetic procedure of the ¹⁷⁷Lu-PSMA I&T in one embodiment.

FIG. 3B is a flowchart of the synthetic procedure of the ¹⁷⁷Lu-PSMA I&T in one embodiment.

FIG. 4 depicts a drawing of an example product vial. The drug product is delivered in a sterile pyrogen free glass vial of Type 1 glass with a fluorocoated bromobutyl rubber septum. The septum is sealed with a crimped aluminum capsule. During transportation, the glass vial containing the radiopharmaceutical is kept in a lead shielded container. The transport container including lead shield and outer packaging complies to type A requirements (IAEA standards).

FIG. 5 shows ¹⁷⁷Lu-PSMA I&T radiochemical purity measured by HPLC at different time points.

FIGS. 6A and 6B show HPLC radio-chromatograms of high radioactivity concentration formulation containing 42.5 mg/ml ascorbic acid at pH of 7±0.1, 0 hours and 71 hours post EOS as detailed in example 3.

FIGS. 7A and 7B show HPLC radio-chromatograms of high radioactivity concentration formulation containing 42.4 mg/ml ascorbic acid at pH of 4.5±0.1, 0 hours and 71 hours post EOS as detailed in example 3.

FIGS. 8A and 8B show HPLC radio-chromatograms of high radioactivity concentration formulation containing 42.5 mg/ml ascorbic acid at pH of 3.5±0.1, 0 hours and 90 hours post EOS as detailed in example 3.

FIGS. 9A and 9B show HPLC radio-chromatograms of low radioactivity concentration formulation containing 21 mg/ml ascorbic acid at pH of 4.5±0.1, 0 hours and 92 hours post EOS as detailed in example 3.

FIGS. 10A and 10B show HPLC radio-chromatograms of low radioactivity concentration formulation containing 31 mg/ml ascorbic acid at pH of 5±0.1, 0 hours and 71 hours post EOS as detailed in example 3.

FIGS. 11A and 11B show HPLC radio-chromatograms of low radioactivity concentration formulation containing 31 mg/ml ascorbic acid at pH of 4.5±0.1, 0 hours and 93 hours post EOS as detailed in example 3.

FIGS. 12A and 12B presents a summary of the kinetics in normal organs as well as tumor lesions for one embodiment of the composition.

FIG. 12C presents a summary of the effective half-lives in normal organs for one embodiment of the composition.

FIG. 12D presents a summary of the mean absorbed doses (presented as medians) in normal organs for one embodiment of the composition.

FIG. 13 presents the baseline and on-treatment disease status evaluation and treatment decisions.

### DETAILED DESCRIPTION

The patent or application file contains at least one drawing executed in color. Copies of this patent or patent application publication with color drawing(s) will be provided by the Office upon request and payment of the necessary fee.

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Disclosed herein is a small molecular inhibitor of PSMA that has the desirable attributes of large monoclonal antibodies with reduced negative aspects, e.g., poor permeability and toxicity. The radiopharmaceutical composition disclosed herein comprises ¹⁷⁷Lu-PSMA I&T. ¹⁷⁷Lu-PSMA I&T is a short-lived radiolabeled substance from which the product is formulated immediately after finished synthesis.

Headings included herein are simply for ease of reference and are not intended to limit the disclosure in any way.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein. All publications, patents and patent applications cited herein, whether supra or infra, are hereby incorporated by reference in their entirety.

### I. Definitions

Several definitions that apply throughout the above disclosure will now be presented. As used herein, the terms “comprising,” “having,” and “including” are used interchangeably in their open, non-limiting sense. The terms “a,” “an,” and “the” are understood to encompass the plural as well as the singular. Thus, the term “a mixture thereof” also relates to “mixtures thereof.”

Generally, the ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, “PSMA” refers to prostate-specific membrane antigen, also known as folate hydrolase I or glutamate carboxypeptidase II, is a type II transmembrane protein, which is anchored in the cell membrane of prostate epithelial cells. PSMA is highly expressed on prostate epithelial cells and strongly up-regulated in prostate cancer. The PSMA expression levels are directly correlated to androgen independence, metastasis, and prostate cancer progression. Thus, PSMA is a promising molecular target for diagnosis and therapy of metastatic prostate cancer at present.

As used herein, “Lutetium-177” and “¹⁷⁷Lu” are used interchangeably. ¹⁷⁷Lu is a β- and γ-emitting radionuclide with a physical half-life of 6.7 days. It has a maximum and mean β-particle energy of 0.498 MeV and 0.133 MeV, respectively. The maximum and mean soft-tissue penetration depth of ¹⁷⁷Lu is 1.7 mm and 0.23 mm, respectively. It has two main gamma emission lines: 113 keV (6% relative abundance) and 208 keV (11% relative abundance).

As used herein, “¹⁷⁷Lu-PSMA-617,” refers to a DOTA derivative of the Glu-urea-Lys motif that has been developed in the German Cancer Research Center (DKFZ) Heidelberg, Germany, for the treatment of patients with metastatic prostate cancer.

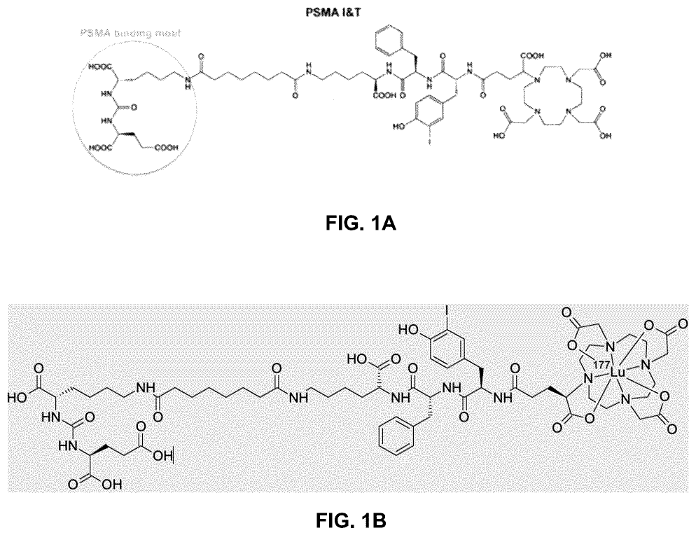

As used herein, “[¹⁷⁷Lu]Lu-PSMA I&T” and “¹⁷⁷Lu-PSMA I&T” refer to ¹⁷⁷Lu-PSMA for imaging and therapy (I&T), a third-generation derivative of ¹⁷⁷Lu-PSMA-compounds which has been used here. The chemical name of ¹⁷⁷Lu-PSMA I&T is (3S,7S,26R,29R,32R,37R)-29-benzyl-32-(4-hydroxy-3-iodobenzyl)-5,13,20,28,31,34-hexaoxo-37-(4,7,10-tris(carboxymethyl)-1,4,7,10-tetraazacyclododecan-1-yl)-4,6,12,21,27,30,33-heptaazaheptatriacontane-1,3,7,26,37-pentacarboxylic acid, lutetium-177 (III). The chemical structure of ¹⁷⁷Lu-PSMA I&T is provided in FIG. 1B.

The term “half-life” as used herein refers to the biological half-life, for example, the time required for a drug's blood or plasma concentration to decrease by one half. This decrease in drug concentration is a reflection of its excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein the term “half-life” also includes the “apparent half-life” of a drug. The apparent half-life may be a composite number that accounts for contributions from other processes besides elimination, such as absorption, reuptake, or enterohepatic recycling.

As used herein, “PRLT” refers to prostate radioligand therapy and “RLT” refers to radioligand therapy. PRLT in this context involves the systemic intravenous administration of a specific radiopharmaceutical composed of a β-emitting radionuclide chelated to a small molecule for the purpose of delivering cytotoxic radiation to cancer cells.

The term “CRPC,” as used herein, refers to castrate resistant prostate cancer. In an example, a patient with CRPC may have castrate serum testosterone <50 μg/l or 1.7 nmol/l plus one of the following types of progression: biochemical progression or radiologic progression, as defined below.

The term “biochemical progression,” as used herein, refers to three consecutive rises in PSA one week apart, resulting in two 50% increases over the nadir, and PSA >2 μg/l.

The term “RAC” as used herein, refers to radioactivity concentration.

The term “radiologic progression,” as used herein, refers to the appearance of new lesions; either two or more new bone lesions on bone scan or a soft tissue lesion using the Response Evaluation Criteria in Solid Tumors (RECIST).

As used herein, the terms “end of synthesis”, “after formulation”, and “end of formulation” are used interchangeably to mean when the process of preparing the composition has completed. This may also include the time after quality control and release of the drug product by a Qualified Person.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “adverse event” (AE) is any untoward medical occurrence in a subject administered an investigational drug, which does not necessarily have a causal relationship with the treatment. An AE can be any unfavorable or unintended sign (e.g., an abnormal laboratory finding), symptom, or disease temporally associated with the use of a drug, whether or not it is considered to be drug related. This includes any newly occurring event or previous condition that has increased in severity or frequency since the administration of the drug.

The terms “subject” or “patient” are used interchangeably herein and refer to a vertebrate, preferably a mammal. Mammals include, but are not limited to, humans.

### II. Introduction

The present disclosure is directed to a radiopharmaceutical composition comprising ¹⁷⁷Lu-PSMA I&T. In some embodiments, the composition may be formulated as a radiopharmaceutical solution for injection. The present disclosure further relates to a high-energy, high purity, and/or low toxicity radiopharmaceutical composition comprising ¹⁷⁷Lu-PSMA I&T that performs as an anti-tumor agent for targeted radionuclide therapy.

The present disclosure is also directed to methods of making the radiopharmaceutical composition. Provided herein are methods of increasing the shelf life of a radiopharmaceutical product.

The present disclosure further relates to the properties of the radiopharmaceutical composition and methods of use of the radiopharmaceutical composition.

¹⁷⁷Lu-PSMA I&T is also known by its synonyms as follows: [¹⁷⁷Lu]Lutetium-PSMA I&T, ¹⁷⁷Lu-ITG-PSMA-1, PSMA-TUM3, ¹⁷⁷Lu-DOTAGA-(I-y)fk(Sub-KuE) or ¹⁷⁷Lu-(3S,7S)-29-benzyl-32(3-iodo, 4-hydroxy)-benzyl-5, 12, 20, 28, 31, 34-hexaoxo-37-(4,7,10-tris(carboxymethyl)-1,4,7,10-tetraazacyclododecan-1-yl)-4, 6, 12, 21, 27, 30, 33-heptaazaheptatriacontane-1,3,7,26,37-pentacarboxylic acid. The molecular formula of the unlabeled precursor is C₆₃H₉₂IN₁₁O₂₃·4TFA·3H₂O with a relative molecular mass of 1498 g/mol.

The labelled substance ¹⁷⁷Lu-PSMA I&T may be labelled with non-carrier-added Lutetium-177 (T_{1/2}=6.6d) solution. ¹⁷⁷Lu-PSMA I&T is a short-lived radiolabeled substance from which the product is formulated immediately after finished synthesis. Therefore, there are no specifications or batch analysis results for the labelled substance. Controls are performed on the labelled drug product.

The synthetized ¹⁷⁷Lu-PSMA I&T solution may be formulated in an injections grade water solution containing stabilizing agents such as ascorbic acid. The solution may be sterilized by aseptic filtration through a 0.22 μm filter prior to dispensing in multidose vials. Administration of the formulated solution may be within 72 h of the end of the synthesis after quality control and release of the drug product by a Qualified Person.

Ascorbic acid may be employed to minimize radiolysis of radiolabeled preparations. In addition to ascorbic acid, dose formulation pH of 5 or below may stabilize the labelled product against radiolytic decomposition and enhancing it shelf life. Thus, in another aspect, the present disclosure further provides a dose formulation containing ascorbic acid at pH of 5 or below that improves stability of the radiopharmaceutical composition against radiolytic decomposition, thus improving the shelf life of the composition.

The stability enhancing conditions may be applied as early as possible in the manufacturing process. For example, ascorbic acid solutions at pH 5 or below may be used instead of water in the purification steps of the labelled ¹⁷⁷Lu-PSMA I&T to minimize radiolytic damage.

The composition, when administered to a subject, may result in low hematotoxicity and nephrotoxicity profiles, providing better effects and fewer adverse effects than monoclonal antibody treatments and other comparable third-line treatments.

The composition is an improved composition in that it has a shelf life of more than 72 hours after formulation. Additionally, the improved composition has a radiochemical purity of greater than 95% at administration. That is, the improved formulation maintains a high level of radiochemical purity more than 72 hours after formulation. Therefore, the improved formulation is suitable for administration up to 24 or up to 72 hours longer than other compositions comprising ¹⁷⁷Lu-PSMA I&T.

### III. Radiopharmaceutical Compositions

The disclosed radiopharmaceutical composition or formulation includes a dose of ¹⁷⁷Lu-PSMA I&T and at least one of a stabilizing agent, an antioxidant, a pH adjuster, a metal ion chelator, water, or a combination thereof.

In one specific embodiment, the stabilizing agent is ethanol. In another embodiment the antioxidant may be ethanol, ascorbic acid, gentisic acid, or a combination thereof. In another embodiment, the pH adjuster includes but is not limited to sodium hydroxide, sodium bicarbonate, hydrochloric acid, or combinations thereof. In yet another embodiment, the chelator may be EDTA or DTPA.

In one embodiment, the medicinal product or radiopharmaceutical composition (or formulation) may be a sterile filtered radiopharmaceutical solution containing a dose of ¹⁷⁷Lu-PSMA I&T in an aqueous ascorbic acid solution containing ethanol. For example, the total amount of ascorbic acid in the solution may be about 25 to about 65 mg/mL and the total amount of ethanol in the solution may be about 3.8% (v/v) to about 7.5% (v/v). In some embodiments, the total amount of ascorbic acid in the solution is from about 21 mg/mL to about 42.5 mg/m L. The ¹⁷⁷Lu-PSMA I&T is present in sufficient amounts of radioactivity for intended use. Experiments performed with various dose formulations suggests that ¹⁷⁷Lu-PSMA I&T formulation composition containing about 31 mg/ml of ascorbic acid at pH of about 4.5 and radioactivity concentration of about 640 MBq/ml or below may provide adequate radiochemical stability of four days. The adequate radiochemical stability referred herein is radiopharmaceutical composition where the radiochemical purity of the ¹⁷⁷Lu-PSMA I&T is at least 95%, 95.5% or greater, 96.5% or greater, 97.0% or greater, 97.5% or greater, 98.0% or greater, 98.5% or greater, 99.0% or greater, or 99.5% or greater at administration.

In one embodiment, the radiopharmaceutical composition is a sterile filtered radiopharmaceutical solution containing a micro dose of ¹⁷⁷Lu-PSMA I&T in an aqueous ascorbic acid and ethanol solution. In another embodiment, the radiopharmaceutical composition is a sterile filtered radiopharmaceutical solution containing a micro dose of ¹⁷⁷Lu-PSMA I&T in an aqueous ascorbic acid without ethanol solution. For example, the radiopharmaceutical composition may be a sterile filtered radiopharmaceutical solution containing a micro dose of ¹⁷⁷Lu-PSMA I&T in an aqueous ascorbic acid and acetate buffer comprising DTPA (e.g., comprising ethanol or in the complete absence of ethanol). The product is diluted to a standard radioactivity concentration and therefore the final volume of the bulk product varies depending on the starting radioactivity of ¹⁷⁷Lu introduced.

One aspect of the disclosure provides for a radiopharmaceutical composition with a pH from about 3 to about 9, from about 4 to about 9, from about 5 to about 9, from about 3 to about 8, from about 4 to about 8, from about 3 to about 5, or from about 5 to about 8. The pH of the radiopharmaceutical composition may be about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, or about 9.

Having a pH of 5 or below may stabilize the radiopharmaceutical composition against radiolytic decomposition and may enhance its shelf life.

In one embodiment, the pH of the radiopharmaceutical composition is from about 3 to about 5. This pH range may stabilize the radiopharmaceutical composition against radiolytic decomposition and may enhance its shelf life. In yet another embodiment, a radiopharmaceutical composition comprising ascorbic acid and having a pH from about 3 to about 5 has improved stability and extended shelf life compared to known radiopharmaceutical compositions of ¹⁷⁷Lu-PSMA I&T which have higher pH values and may comprise gentisic acid.

The pH of the radiopharmaceutical composition may range from 3.0 to 5.0, 3.0 to 3.5, 3.0 to 3.05, 3.05 to 3.1, 3.0 to 3.1, 3.1 to 3.15, 3.1 to 3.2, 3.15 to 3.2, 3.2 to 3.25, 3.0 to 3.25, 3.2 to 3.3, 3.25 to 3.3, 3.3 to 3.35, 3.3 to 3.4, 3.35 to 3.4, 3.4 to 3.45, 3.4 to 3.5, 3.45 to 3.5, 3.25 to 3.5, 3.5 to 3.55, 3.5 to 3.6, 3.55 to 3.6, 3.6 to 3.65, 3.6 to 3.7, 3.65 to 3.7, 3.7 to 3.75, 3.5 to 3.75, 3.7 to 3.8, 3.75 to 3.8, 3.8 to 3.85, 3.8 to 3.9, 3.85 to 3.9, 3.9 to 3.95, 3.9 to 4.0, 3.95 to 4.0, 3.5 to 4.0, 3.75 to 4.0, 4.0 to 4.05, 4.0 to 4.1, 4.05 to 4.1, 4.1 to 4.15, 4.1 to 4.2, 4.15 to 4.2, 3.5 to 4.2, 4.2 to 4.25, 4.0 to 4.25, 4.2 to 4.3, 4.25 to 4.3, 4.3 to 4.35, 4.3 to 4.4, 4.35 to 4.4, 4.4 to 4.45, 4.4 to 4.5, 4.45 to 4.5, 4.25 to 4.5, 4.0 to 4.5, 4.5 to 4.55, 4.5 to 4.6, 4.55 to 4.6, 4.6 to 4.65, 4.6 to 4.7, 4.65 to 4.7, 4.7 to 4.75, 4.7 to 4.8, 4.75 to 4.8, 4.8 to 4.85, 4.8 to 4.9, 4.85 to 4.9, 4.9 to 4.95, 4.9 to 5.0, 4.95 to 5.0, 4.5 to 5.0, or 4.75 to 5.0. In some examples, the pH of the radiopharmaceutical composition may be adjusted to a final pH of 3.0, 3.5, 4.0, 4.5, or 5.0. In some embodiments, including the above listed pH numbers and ranges, the pH values are inclusive of ±0.05, 0.10, 0.15, 0.20, or 0.25.

In another embodiment, the radiopharmaceutical composition or formulation has a purity of at least about 90%, at least about 95%, or at least about 99%. In another embodiment, the radiopharmaceutical composition or formulation has a purity of about 90%, about 91%, about 92%, about 93%, about 94%, about 95%, about 96%, about 97%, about 98%, about 99%, or about 99.5%.

In another embodiment, radiopharmaceutical composition or formulation has a purity of at least about 90%, at least about 95%, or at least about 99% as measured by HPLC, TLC, or liquid chromatography. In another embodiment, the radiopharmaceutical composition or formulation has a purity of at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, or at least about 99.5% as measured by HPLC, TLC, or liquid chromatography. In some examples, the radiopharmaceutical composition may have a radiochemical purity of 95.0% or greater, 95.5% or greater, 96.0% or greater, 96.5% or greater, 97.0% or greater, 97.5% or greater, 98.0% or greater, 98.5% or greater, 99.0% or greater, or 99.5% or greater at administration. FIG. 5 shows the radiochemical purity at different pH formulations.

In another embodiment, the purity of the radiopharmaceutical composition or formulation is measured by HPLC, TLC, or liquid chromatography at any time post end of synthesis (EOS). In one embodiment, the purity of the radiopharmaceutical composition or formulation is measured by HPLC, TLC, or liquid chromatography at about 0 hour, about 10 hours, about 20 hours, about 30 hours, about 40 hours, about 50 hours, about 50 hours, about 60 hours, about 70 hours, about 80 hours, about 90 hours, about 100 hours post EOS.

In one specific embodiment, the radiopharmaceutical composition or formulation has a purity of at least about 99% as measured by HPLC, TLC, or liquid chromatography 0 hours post EOS. In another specific embodiment, the radiopharmaceutical composition or formulation has a purity of at least about 96.5% as measured by HPLC, TLC, or liquid chromatography 24 hours post EOS, at least about 93% as measured by HPLC, TLC, or liquid chromatography 46 hours post EOS, at least about 95% as measured by HPLC, TLC, or liquid chromatography 67 hours post EOS, at least about 96% as measured by HPLC, TLC, or liquid chromatography 92 hours post EOS.

In another embodiment, the radioactivity is measured in a dose calibrator. The radioactive amount of ¹⁷⁷Lu-PSMA I&T is determined when the dose is dispensed.

In yet another embodiment, the radiochemical purity of ¹⁷⁷Lu-PSMA I&T is determined by liquid chromatography with radioactivity detection and thin layer chromatography.

In one embodiment, bacterial endotoxin content is determined for each batch before release using a PTS-tester (Ph Eur method D) and sterility is determined according to Ph Eur.

In one embodiment, the radiopharmaceutical composition or formulation is stored at a temperature from about +5° C. to +40° C., about +10° C. to +35° C. or about +20° C. to +30° C. In one specific embodiment, the radiopharmaceutical composition or formulation is stored at a temperature at about +10° C., about +15° C., about +22° C., about +22.5° C., about +25° C., or at room temperature.

Another aspect of the disclosure provides for a radioactive content of about 70% to 130%. The radioactive content of the radiopharmaceutical composition may be about 70% to 125%, 70% to 120%, 70% to 115%, 70% to 110%, 80% to 130%, 85% to 130%, 90% to 130%, 95% to 130%, 75% to 125%, 75% to 120%, 75% to 115%, 75% to 110%, 80% to 125%, 80% to 120%, 80% to 115%, 80% to 110%, 85% to 125%, 85% to 120%, 85% to 115%, 85% to 110%, 90% to 125%, 90% to 120%, 90% to 115%, or 90% to 110%.

In one specific embodiment, the radioactive content of the formulation is about 70%, about 75%, about 80%, about 85%, about 90%, about 95%, about 100%, about 105%, about 110%, about 115%, about 120%, about 125%, or about 130%.

Another aspect of the disclosure provides for a radiopharmaceutical composition with a mean whole-body effective dose of about 23±20 Gy (3.3 Gy/GBq), with a mean absorbed organ doses of about 26±20 Gy (3.4 Gy/GBq), 24±16 Gy (3.2 Gy/GBq), 8.5±4.7 Gy (1.28 Gy/GBq), and 13±7.4 Gy (1.7 Gy/GBq) for the bone, lymph node, liver, and lung metastases, respectively.

In some embodiments, the radiopharmaceutical composition may have a low radioactivity concentration (“low RAC”) of about 579 MBq/ml to about 626 MBq/ml. For example, the radiopharmaceutical composition may have a low radioactivity that may be about 11,580 MBq (313 mCi), about 11,770 MBq (318 mCi), or about 12,520 MBq (338 mCi) in a 20 ml volume of the solution. In other embodiments the radiopharmaceutical composition may have a low radioactivity concentration of at least about 550 MBq/ml, at least about 560 MBq/ml, at least about 570 MBq/ml, at least about 580 MBq/ml, at least about 590 MBq/ml, at least about 600 MBq/ml, at least about 610 MBq/ml, at least about 620 MBq/ml, at least about 630 MBq/ml, at least about 640 MBq/ml, or at least about 650 MBq/ml. In still other embodiments the radiopharmaceutical composition may have a low radioactivity concentration from about 550 MBq/ml to about 575 MBq/ml, from about 575 MBq/ml to about 600 MBq/ml, from about 600 MBq/ml to about 625 MBq/ml, or from about 625 MBq/ml to about 650.

In additional embodiments, the radiopharmaceutical composition may have a high radioactivity concentration (“High RAC”) of about 1,270 MBq/ml to about 1,311 MBq/ml. For example, the radiopharmaceutical composition may have a high radioactivity that may be about 12,780 MBq (345 mCi), about 12,810 MBq (346 mCi), or about 13,110 MBq (354 mCi) in a 10 ml volume of the solution. In other embodiments the radiopharmaceutical composition may have a high radioactivity concentration of at least about 1,100 MBq/ml, at least about 1,110 MBq/ml, at least about 1,120 MBq/ml, at least about 1,130 MBq/ml, at least about 1,140 MBq/ml, at least about 1,150 MBq/ml, at least about 1,160 MBq/ml, at least about 1,170 MBq/ml, at least about 1,180 MBq/ml, at least about 1,190 MBq/ml, at least about 1,200 MBq/ml, 1,200 MBq/ml, at least about 1,210 MBq/ml, at least about 1,220 MBq/ml, at least about 1,230 MBq/ml, at least about 1,240 MBq/ml, at least about 1,250 MBq/ml, at least about 1,260 MBq/ml, at least about 1,270 MBq/ml, at least about 1,280 MBq/ml, at least about 1,290 MBq/ml, at least about 1,300 MBq/ml, at least about 1,310 MBq/ml, at least about 1,320 MBq/ml, at least about 1,330 MBq/ml, at least about 1,340 MBq/ml, or at least about 1,350 MBq/ml. In still other embodiments the radiopharmaceutical composition may have a high radioactivity concentration from about 1,000 MBq/ml to about 1,400 MBq/ml, from about 1,050 MBq/ml to about 1,350 MBq/ml, from about 1,100 MBq/ml to about 1,300 MBq/ml, from about 1,150 MBq/ml to about 1,250 MBq/ml, from about 1,200 MBq/ml to about 1,300 MBq/ml, from about 1,250 MBq/ml to about 1,350 MBq/ml, or from about 1,250 MBq/ml to about 1,300 MBq/ml.

(i) ¹⁷⁷Lu-PSMA I&T

The total amount of ¹⁷⁷Lu-PSMA I&T present in the radiopharmaceutical composition can and will vary. FIGS. 1A and 1B show the chemical structure of precursor PSMA I&T and ¹⁷⁷Lu-PSMA I&T respectively.

In one embodiment, the mass of radioactive pharmaceutical ingredient (¹⁷⁷Lu-PSMA I&T) in the drug product is less than about 30 μg, less than about 25 μg, less than about 20 μg, less than about 15 μg, or less than about 10 μg per vial. In yet another embodiment, the mass of radioactive pharmaceutical ingredient (¹⁷⁷Lu-PSMA I&T) in the drug product is about 1 μg, about 2 μg, about 3 μg, about 4 μg, about 5 μg, about 6 μg, about 7 μg, about 8 μg, about 9 μg, or about 10 μg, about 11 μg, about 12 μg about, about 13 μg, about 14 μg, about 15 μg, about 16 μg, about 17 μg, about 18 μg, about 17.2 μg, about 18 μm about 19 μg, about 20 μg, about 21 μg, about 22 μg, about 23 μg, about 24 μg, about 25 μg, about 26 μg, about 27 μg, about 28 μg, about 29 μg, or about 30 μg of ¹⁷⁷Lu-PSMA I&T per vial.

In some embodiments, the total amount of ¹⁷⁷Lu-PSMA I&T present in the radiopharmaceutical composition may range from about 1.0 μg/ml to about 3 μg/ml, from about 1 μg/ml to about 2 μg/ml, from about 1.1 μg/ml to about 2 μg/ml, from about 1.1 μg/ml to about 1.5 μg/ml, from about 1.1 μg/ml to about 1.4 μg/ml, or from about 1.1 μg/ml to about 1.3 μg/ml. In another embodiment, the total amount of ¹⁷⁷Lu-PSMA I&T in the radiopharmaceutical composition may range from about 0.5 μg/ml to about 1.5 μg/ml. In various embodiments, the total amount of ¹⁷⁷Lu-PSMA I&T present in the radiopharmaceutical composition may be about 0.5 μg/ml, about 0.6 μg/ml, about 0.7 μg/mi, about 0.8 μg/ml, about 0.9 μg/ml, about 1.0 μg/ml, about 1.1 μg/ml, about 1.2 μg/mi, about 1.3 μg/ml, about 1.4 μg/ml, about 1.5 μg/ml, about 1.6 μg/ml, about 1.7 μg/mi, or about 1.8 μg/ml.

In some embodiments, the total amount of ¹⁷⁷Lu-PSMA I&T present in the radiopharmaceutical composition may range from about 3.0 μg/ml to about 9.0 μg/mi, from about 3.5 μg/ml to about 8.5 μg/ml, from about 4.0 μg/ml to about 8.0 μg/ml, from about 4.5 μg/ml to about 7.5 μg/ml, from about 5.0 μg/ml to about 7.0 μg/ml, or from about 5.5 μg/ml to about 6.5 μg/ml. In another embodiment, the total amount of ¹⁷⁷Lu-PSMA I&T in the radiopharmaceutical composition may range from about 0.5 μg/mi to about 1.5 μg/mi.

In some embodiments, the total amount of ¹⁷⁷Lu-PSMA I&T present in the radiopharmaceutical composition may be less than 3.0 μg/ml. In other embodiments, the total amount of ¹⁷⁷Lu-PSMA I&T present in the radiopharmaceutical composition may be less than 4.0 μg/ml. In other embodiments, the total amount of ¹⁷⁷Lu-PSMA I&T present in the radiopharmaceutical composition may be less than 5.0 μg/ml. In other embodiments, the total amount of ¹⁷⁷Lu-PSMA I&T present in the radiopharmaceutical composition may be less than 6.0 μg/ml.

In some embodiments, the total amount of ¹⁷⁷Lu-PSMA I&T present in the radiopharmaceutical composition may range from about 9 μg/ml to 20 μg/ml, 10 μg/ml to 20 μg/ml, 11 μg/ml to 20 μg/ml, 11 μg/ml to 15 μg/ml, 11 μg/ml to 14 μg/ml, or 11 μg/ml to 13 μg/ml. In another embodiment, the total amount of ¹⁷⁷Lu-PSMA I&T in the radiopharmaceutical composition may range from about 5 μg/ml to about 15 μg/ml. In various embodiments, the total amount of ¹⁷⁷Lu-PSMA I&T present in the radiopharmaceutical composition may be about 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, or 18 μg/ml. The composition may have less than 12 μg/ml or less than 6 μg/ml of Lu-PSMA I&T.

The radioactivity/volume of ¹⁷⁷Lu-PSMA I&T in the composition may be adjusted according to dose strength. In an embodiment, the composition may include 0.5 GBq (13.5 mCi) of ¹⁷⁷Lu-PSMA I&T in a 1 ml solution. In other words, the composition may include 10 GBq (270 mCi) of ¹⁷⁷Lu-PSMA I&T in a 20 ml solution. In another embodiment, the composition may include 1 GBq (27 mCi) of ¹⁷⁷Lu-PSMA I&T in a 1 ml solution. In other words, the composition may include 10 GBq (270 mCi) of ¹⁷⁷Lu-PSMA I&T in a 10 ml solution.

In one embodiment, the radioactivity concentration of the ¹⁷⁷Lu-PSMA I&T in the radiopharmaceutical composition is less than about 50 mCi/ml, less than about 45 mCi/ml, less than about 40 mCi/ml, less than about 35 mCi/ml, less than about 30 mCi/ml, less than about 25 mCi/ml, less than about 20 mCi/ml, or less than about 15 mCi/ml. In another embodiment, the radioactivity concentration of the ¹⁷⁷Lu-PSMA I&T in the radiopharmaceutical composition is from about 5 mCi/ml to about 30 mCi/ml, from about 10 mCi/ml to about 20 mCi/ml, or from about 13 mCi/ml to about 30 mCi/ml. In one specific embodiment, the radioactivity concentration of the ¹⁷⁷Lu-PSMA I&T in the radiopharmaceutical composition is about 5 mCi/ml, about 10 mCi/ml, about 13.5 mCi/ml, about 15 mCi/ml, about 20 mCi/ml, about 27 mCi/ml, about 30 mCi/ml, about 30 mCi/ml, about 35 mCi/ml or about 40 mCi/ml.

In one embodiment, the radioactivity of the ¹⁷⁷Lu-PSMA I&T in the radiopharmaceutical composition is less than about 500 mCi, less than about 450 mCi, less than about 400 mCi, less than about 350 mCi, less than about 300 mCi, less than about 250 mCi, or less than about 200 mCi per vial. In another embodiment, the radioactivity of the ¹⁷⁷Lu-PSMA I&T in the radiopharmaceutical composition is from about 10 mCi to about 750 mCi, from about 200 mCi to about 600 mCi, from about 300 mCi to about 400 mCi per vial. In one specific embodiment, the radioactivity of the ¹⁷⁷Lu-PSMA I&T in the radiopharmaceutical composition is about 27 mCi, 150 mCi, about 160 mCi, about 170 mCi, about 180 mCi, about 190 mCi, about 200 mCi, about 250 mCi, about 270 mCi, about 300 mCi, about 313 mCi, about 318 mCi, about 338 mCi, about 345 mCi, about 346 mCi, about 354 mCi, about 360 mCi, about 370 mCi, about 380 mCi, about 390 mCi, about 400 mCi, about 450 mCi, about 500 mCi, about 550 mCi, about 600 mCi or about 700 mCi per vial.

In yet another embodiment, the ¹⁷⁷Lu-PSMA I&T drug product has a standard radioactivity concentration of about 12 mCi/ml or about 32 mCi/ml at the end of production. In one embodiment, the ¹⁷⁷Lu-PSMA I&T drug product has standard radioactivity concentration of about 13.5 mCi/ml or about 27 mCi/ml at the end of production.

(ii) Antioxidant

The antioxidant may act as a buffer and/or stabilizing agent. The total amount of antioxidant in the radiopharmaceutical composition can and will vary. Examples of suitable antioxidants include but are not limited to ascorbic acid or gentisic acid. The amount of antioxidant in the composition may range from about 10 mg/ml to 90 mg/ml, about 15 mg/ml to 85 mg/ml, about 20 mg/ml to 80 mg/ml, about 25 mg/ml to 75 mg/ml, about 30 mg/ml to 70 mg/ml, about 35 mg/ml to 65 mg/ml, about 40 mg/ml to 60 mg/ml, or about 45 mg/ml to 55 mg/ml. Said in another way, the amount of antioxidant in the composition may range from about 10 mg to 90 mg, about 15 mg to 85 mg, about 20 mg to 80 mg, about 25 mg to 75 mg, about 30 mg to 70 mg, about 35 mg to 65 mg, about 40 mg to 60 mg, or about 45 mg to 55 mg per ml.

In an embodiment, the antioxidant may be ascorbic acid. Ascorbic acid may minimize or reduce radiolysis of radiolabeled compositions.

In some embodiments, ascorbic acid present in the radiopharmaceutical composition may range from about 10 mg to about 90 mg, from about 10 to about 80 mg, from about 10 to about 70 mg, from about 10 to about 60 mg, from about 10 to about 50 mg, from about 20 to about 50 mg, from about 30 to about 50 mg or from about 35 to about 45 mg per ml. In another embodiment, ascorbic acid in the radiopharmaceutical composition may range from about 5 mg to about 50 mg per ml.

In various embodiments, ascorbic acid present in the radiopharmaceutical composition may be about 5 mg, about 10 mg, about 15 mg, about 20 mg, about 25 mg, about 30 mg, about 31 mg, about 35 mg, about 36 mg, about 37 mg, about 38 mg, about 39 mg, about 40 mg, about 40.5 mg, about 41 mg, about 41.5 mg, about 42 mg, about 42.5 mg, about 43 mg, about 43.5 mg, about 44 mg, about 44.5 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, or about 90 mg per ml. For example, the amount of ascorbic acid in 1 ml of the composition may be about 25 mg to 30 mg, about 30 mg to 35 mg, about 35 mg to 40 mg, or about 40 mg to 45 mg.

In yet another embodiment, the concentration of ascorbic acid in the radiopharmaceutical composition may be from about 10 mg/ml to about 80 mg/ml, from about 10 mg/ml to about 75 mg/mi, from about 10 mg/ml to about 70 mg/mi, from about 15 mg/ml to about 80 mg/ml, from about 15 mg/ml to about 75 mg/ml, from about 15 mg/ml to about 70 mg/ml, from about 20 mg/ml to about 80 mg/ml, from about 20 mg/ml to about 75 mg/mi, or from about 20 mg/ml to about 70 mg/mi.

In one specific embodiment, the concentration of ascorbic acid in the radiopharmaceutical composition is about 10 mg/ml, about 15 mg/ml, about 20 mg/ml, about 25 mg/ml, about 21 mg/ml, about 25 mg/ml, about 30 mg/ml, about 31 mg/ml, about 35 mg/ml, about 40 mg/ml, about 42.5 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, or about 100 mg/ml.

In at least one embodiment, the total amount of ascorbic acid in the radiopharmaceutical composition may be about 31 mg/ml. In a further embodiment, the total amount of ascorbic acid in the radiopharmaceutical composition may be about 15 mg/ml, about 21 mg/ml, about 25 mg/ml, about 31 mg/ml, or about 42.5 mg/ml.

(iii) Stabilizing Agent

The stabilizing agent may be separate from the antioxidant. The total amount of stabilizing agent present in the radiopharmaceutical composition can and will vary. The stabilizing agent may further be used to limit or reduce radiolysis. The stabilizing agent may also function as a vehicle for the composition.

The stabilizing agent includes but is not limited to ethanol, para-aminebenzoic acid (PABA), dihydroxybenzoic acid (gentisate compounds), gentisic acid, cysteine, selenomethionine, ascorbic acid/sodium ascorbate, methionine, and combinations thereof.

In some embodiments, the stabilizing agent is ethanol. Ethanol may be present in the pharmaceutical composition at about 0.01% (v/v) to about 10% (v/v), 0.01% (v/v) to 3% (v/v), about 0.5% (v/v) to 1% (v/v), about 1% (v/v) to 2% (v/v), about 2% (v/v) to about 3% (v/v), about 3% (v/v) to 4% (v/v), about 3.5% to 4.5% (v/v), about 4% to 5% (v/v), about 4.5% (v/v) to 5.5% (v/v), about 5% (v/v) to 6% (v/v), about 5.5% (v/v) to 6.5% (v/v), about 6% (v/v) to 7% (v/v), about 6.5% (v/v) to 7.5% (v/v), or about 7% (v/v) to 8% (v/v). In some embodiments, the pharmaceutical composition comprises zero (0.00% v/v) ethanol (i.e., ethanol may be absent from the pharmaceutical composition).

In one embodiment, the total amount of ethanol present in the radiopharmaceutical composition is from about 3% (v/v) to about 8% (v/v), or from 2% (v/v) to about 4% (v/v), or from about 7% (v/v) to about 8% (v/v). In various embodiments, the total amount of ethanol present in the radiopharmaceutical composition may be about 1% (v/v), about 2% (v/v), about 3% (v/v), about 3.5% (v/v), about 3.8% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v).

In at least one example, the radiopharmaceutical composition includes 3.8% (v/v) ethanol. In another example, the radiopharmaceutical composition includes 7.5% (v/v).

Stated alternatively, the total amount of ethanol present in the radiopharmaceutical composition may be about 5 mg, about 10 mg, about 15 mg, about 20 mg, about 25 mg, about 30 mg, about 35 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 95 mg, about 100 mg, about 105 mg, about 110 mg, about 115 mg, or about 120 mg per ml.

In some embodiments, the total amount of ethanol present in the radiopharmaceutical composition may range from about 20 mg to about 35 mg per ml. In another embodiment, the total amount of ethanol in the radiopharmaceutical composition may range from about 43 mg to about 63 mg per ml.

In some embodiments, the total amount of ethanol present in the radiopharmaceutical composition may range from about 25 mg to 80 mg, about 30 to 40 mg, about 40 to 50 mg, about 50 to 60 mg, about 60 to 70 mg, or about 70 to 80 mg per. In another embodiment, the total amount of ethanol in the radiopharmaceutical composition may range from about 30 mg to about 60 mg per ml.

In a further embodiment, the ratio of ethanol in the radiopharmaceutical composition may be about 300 mg per 10 ml or about 30 mg/ml. In another embodiment, the ratio of ethanol in the radiopharmaceutical composition may be about 200 mg per 10 ml. In still another embodiment, the ratio of ethanol in the radiopharmaceutical composition may be about 350 mg per 10 ml.

Stated alternatively, the amount of ethanol in the composition may range from about 35 μl/ml to about 75 μl/ml. For example, the amount of ethanol in 1 ml of the composition may be about 35 μl to 40 μl, about 40 μl to 45 μl, about 45 μl to 50 μl, about 50 μl to 55 μl, about 55 μl to 60 μl, about 60 μl to 65 μl, about 65 μl to 70 μl, or about 70 μl to 75 μl. In at least one example, 1 ml of the composition includes 37.5 μl (29.5 mg) of ethanol. In another example, 1 ml of the composition includes 75 μl (58.9 mg) of ethanol.

(iv) Metal Ion Chelator (Chelating Agent)

In some embodiments, the disclosure provides for a radiopharmaceutical composition with a micro dose of ¹⁷⁷Lu-PSMA I&T solution and at least one metal ion chelator. A suitable chelating agent may include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA.

In some embodiments, the metal ion chelator may be ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the metal ion chelator may be disodium EDTA. In one embodiment, the metal ion chelator may be DPTA.

In one embodiment, the amount of chelating agent present in the radiopharmaceutical composition may range from about 5 μg to 500 μg. In some embodiments, the amount of metal ion chelator present in the radiopharmaceutical composition may range from about 5 μg to 50 μg.

In some embodiments, the amount of chelating agent present may be about 5 μg, about 6 μg, about 7 μg, about 8 μg, about 9 μg, about 10.5 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 45 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, or about 500 μg.

The concentration of metal ion chelator in the composition may range from about 5 μg/ml to about 500 μg/ml. In another embodiment, the concentration of chelating agent present in the radiopharmaceutical composition may range from about 5 μg/ml to 75 μg/ml, 10 μg/ml to about 25 μg/ml, about 25 μg/ml to about 50 μg/ml, about 50 μg/ml to about 75 μg/ml, or about 75 μg/ml to about 100 μg/ml. In some embodiments, the concentration of chelating agent present may be about 5 μg/ml, about 6 μg/ml, about 7 μg/ml, about 8 μg/ml, about 9 μg/ml, about 10.5 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, about 20 μg/ml, about 21 μg/ml, about 22 μg/mi, about 23 μg/ml, about 24 μg/ml, about 25 μg/ml, about 26 μg/ml, about 27 μg/ml, about 28 μg/ml, about 29 μg/ml, about 30 μg/ml, about 31 μg/ml, about 32 μg/ml, about 33 μg/ml, about 34 μg/ml, about 35 μg/ml, about 36 μg/ml, about 37 μg/ml, about 38 μg/mi, about 39 μg/ml, about 40 μg/ml, about 45 μg/ml, or about 50 μg/ml.

In another embodiment, the amount of metal ion chelator in the radiopharmaceutical composition may be from about 0.001% to about 0.20% (w/w), about 0.20% to about 0.40% (w/w), about 0.40% to about 0.60% (w/w), about 0.60% to about 0.80% (w/w), or about 0.80% to about 1.00% (w/w) of such radiopharmaceutical composition. In some embodiments, the amount of metal ion chelator present in a radiopharmaceutical composition may be about 0.001%, 0.002%, 0.003%, 0.004%, 0.005%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of the radiopharmaceutical composition.

For example, the amount of disodium EDTA, diethylenetriamine-pentaacetic acid (DTPA), or a combination thereof in 1 ml of the composition may be about 10 μg to 15 μg, about 13 μg to 18 μg, about 15 μg to 20 μg, about 20 μg to 25 μg, about 25 μg to 50 μg, about 50 μg to 75 μg, or about 75 μg to 150 μg. In some embodiments, the amount of disodium EDTA present may be about 5 μg, about 6 μg, about 7 μg, about 8 μg, about 9 μg, about 10.5 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 45 μg, or about 50 μg.

In at least one example, 1 ml of the composition includes 15.5 μg of disodium EDTA. In another example, 1 ml of the composition includes 21 μg of disodium EDTA.

(v) pH adjuster

Suitable pH adjusters include but are not limited to any one of hydrochloric acid, sodium hydroxide, sodium bicarbonate, or combinations thereof.

In some embodiments, hydrochloric acid may be used to adjust the pH of the radiopharmaceutical composition. In an embodiment, the amount of hydrochloric acid in the composition may range from 0 mg/ml to about 2 mg/ml. In some embodiments, the amount of HCl may range from 1.6 ml of 0.05 M HCl to 2 ml of 0.04 M HCl. The amount of HCl in the composition may vary, to adjust the final formulation pH. In various embodiments, the final formulation pH ranges from pH 3.0 to 5.0. In at least one example, HCl is added to the composition until a final pH of 3.5±0.1 to 4.5±0.1 is reached.

In an embodiment, the amount of sodium bicarbonate in the composition may be a sufficient quantity control the pH of the composition to 5.5 to 7.0 prior to the addition of HCl.

In an embodiment, the amount of NaOH in the composition may be a sufficient quantity control the pH of the composition to 5.5 to 7.0 prior to the addition of HCl.

(vi) Water

The composition may further include a sufficient amount of water to make the desired final volume for the solution for injection. For example, water may be added to make a final volume of 1 ml, 10 ml, or 20 ml. The 10 ml or 20 ml solutions may be stored in a vial and divided into smaller volumes for administration.

### IV. Process of Making the Radiopharmaceutical Composition

The whole manufacturing process is a one-step radiolabelling process using a PSMA I&T precursor. The success of the labelling is depending on temperature, time and pH. The reaction takes place in a reactor vial at elevated temperatures. For example, the reactor maybe heated at setpoint of 110° C. and the maximum temperature reached in the reaction solution is about 95° C. The radiolabeled product is isolated on a C18 cartridge and formulated to the final composition after elution to the bulk vial. The final product is dispensed in a grade A controlled environment.

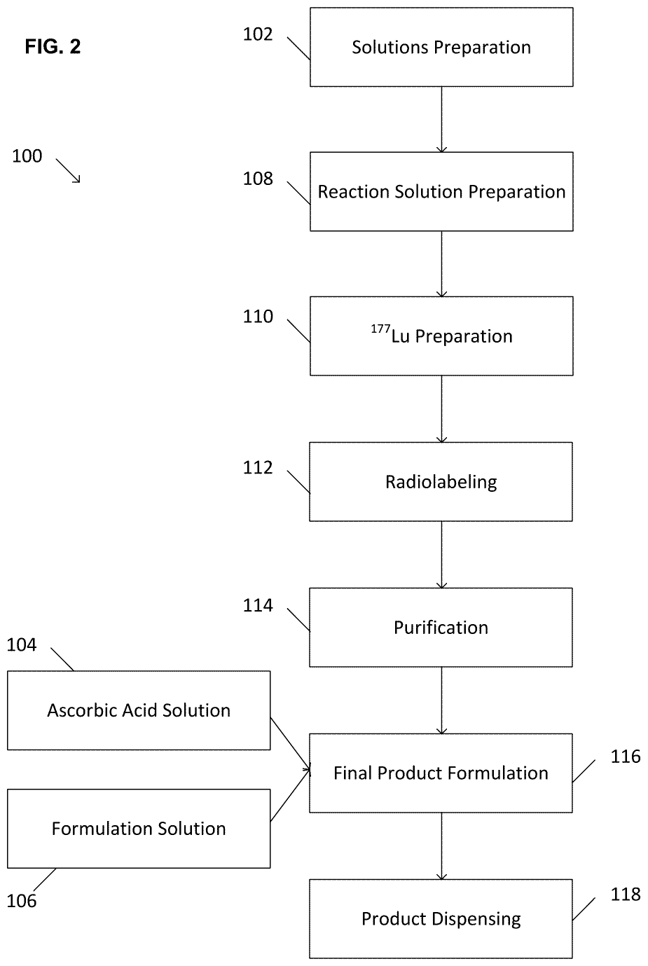

The ¹⁷⁷Lu-PSMA I&T composition solution may be prepared using the following method 100, for example as shown in FIG. 2. The order of the steps may vary, such as the order of preparing the various solutions.

In an embodiment, step 102 may include preparing four solutions for the synthesis. The four solutions may include 0.04 M hydrochloric acid, 0.4 M sodium acetate, 20% (w/w) L-ascorbic acid, and PSMA I&T in water at about 460 to about 500 μg/ml. The PSMA I&T precursor may be dissolved in sterile water for injection. For example, 120 μg to 600 μg of precursor may be used in the reaction depending on the number of doses produced. In at least one example, 463 μl/ml of PSMA I&T precursor may be used to produce the composition.

In an embodiment, step 104 may include preparing an ascorbic acid solution (dilution buffer). In some examples, the ascorbic acid solution may be a 50 mg/ml ascorbic acid solution. The solution pH may be adjusted to 4.5±0.25. For example, 50 mg/ml ascorbic acid solution is prepared, and the pH of the solution is adjusted to 4.5 using 30% hydrochloric acid. In another example, the ascorbic acid solution may include 33 mg/ml ascorbic acid/sodium ascorbate at pH 4.25±0.25 and 0.1 mg/ml DTPA.

In an embodiment, optional step 106 may include preparing a formulation solution/buffer. The formulation solution is prepared from an injections grade solution containing ascorbic acid, absolute ethanol and injections grade water. In an example, the formulation solution is prepared by adding sufficient amounts of following solutions into the bulk vial: about 50 mg/ml ascorbic acid pH 4.5 solution (prepared in step 104), 30% ethanol solution, and water. The formulation solution may include 31 mg/ml to 42.5 mg/ml ascorbic acid and 3.8% to 7.5% ethanol (v/v %). In some embodiments, the formulation buffer may be adjusted to enable the final composition to have an extended shelf life. In at least one example, the formulation solution comprises 31 mg/ml ascorbic acid, 3.8% (v/v) ethanol, and pH 4.5. The formulation buffer is prepared ex tempore as part of the synthesis preparation and predetermined amount is added to the bulk vial as part of the synthesis preparations.

In an embodiment, step 108 may include preparing a reaction solution. A reaction solution may include sodium acetate, HCl, and L-ascorbic acid. Alternatively, a reaction solution may include sodium ascorbate. The reaction solution may be prepared in the reactor using solutions prepared in step 102. In an example, the reaction solution may include 4 ml 0.4 M sodium acetate, a volume of about 463 μg/ml PSMA I&T solution, and 150 μl 20% (w/w) L-ascorbic acid. In another example, the reaction solution may include 0.33M sodium ascorbate (reaction buffer) and PSMA I&T in the reaction buffer. In some examples, the reaction solution may include 1.6 ml 0.05 M HCl or 2 ml 0.04 M HCl (0.08 mmol HCl). Ascorbic acid concentration in the reaction solution may range from 3.75 mg/ml to 5.00 mg/ml.

In an embodiment, step 110 may include preparing ¹⁷⁷Lu. In some embodiments, ¹⁷⁷Lu may be provided in HCl. [¹⁷⁷Lu]LuCl₃ may be provided in 0.04 M or 0.05 M HCl. For example, 40-44 GBq/ml of ¹⁷⁷Lu may be provided in 0.04 M HCl. In another example, less than 61 GBq of ¹⁷⁷Lu may be provided in 0.05 M HCl. The [¹⁷⁷Lu]LuCl₃ in 0.04 M or 0.05 M hydrochloric acid may be transferred into the reactor and the [¹⁷⁷Lu]LuCl₃ vial may be rinsed with an additional required volume of 0.04 M hydrochloric acid (prepared in step 102) that is also then transferred into the reactor.

The reaction volume may range from 6 ml to 8 ml. The volume may be dependent on the amount of precursor used.

In an embodiment, step 112 may include radiolabeling the PSMA-I&T with ¹⁷⁷Lu. The reaction mixture may be heated up to about 75° C., up to about 80° C., up to about 85° C., up to about 90° C., or up to about 95° C. In an example, the setpoint for heating is 110° C. and the actual maximum temperature reached is about 95° C. The reaction volume may be heated for up to 5 minutes, up to 10 minutes, up to 15 minutes, or up to 20 minutes. In at least one example, the reaction mixture is heated at setpoint of 110° C. for 15 minutes. In at least one additional example, the reaction mixture is heated at a setpoint of 75° C. for 10 minutes.

In an embodiment, optional step 114 may include purifying the reaction mixture. For example, the solution may be run through a cassette/cartridge containing a hydrophobic, reverse-phase, silica-based bonded phase. A Sep-Pak C18 may be used to purify the composition. In at least one example, the reaction mixture may be passed through a C18 Sep Pak cartridge and the cartridge is rinsed with water. ¹⁷⁷Lu-PSMA I&T product is retained in the cartridge. In some embodiments, the reaction mixture may not be purified.

In an embodiment, step 116 may include eluting or diluting the final product. In an example, the ¹⁷⁷Lu-PSMA I&T is diluted with dilution buffer prepared in step 104 to the desired radioactivity concentration. The composition may be eluted using 1.5 ml of ethanol-water in a 1:1 ratio. The cassette may then be flushed using 8.5 ml ascorbic acid 50 mg/ml. A formulation solution may then be added to form the final composition. In at least one example, ¹⁷⁷Lu-PSMA I&T is eluted from the C18 cartridge with 1.5 ml of 50% (v/v) ethanol followed by 8.5 ml of 50 mg/ml pH 4.5 ascorbic acid solution (prepared in step 104) into the bulk vial where it is diluted with formulation solution/buffer (prepared in step 106, already in the bulk vial). The resulting solution may have a pH of 3.5 to 4.5. In some embodiments, the pH may be adjusted. In an example, the 50 mg/ml ascorbic acid solution pH is adjusted to 3.5 to 4.5. In other examples the pH is adjusted to 5.0 or less.

Presumably the stability enhancing conditions, such as ascorbic acid solution at pH of about 5 or below, should preferably be applied as early as possible in the process. For example, ascorbic acid solutions at pH 5 or below may be used instead of water at step 114 to minimize radiolytic damage.

In some embodiments, at step 118 the final composition may be sterile filtered. The sterile filter may be a 0.22 μm sterile filter. The final product may be dispensed through a 0.22 μm sterile filter into single dose vials containing suitable volume and radioactivity referenced to the prescribed calibration time. For example, the final composition may be dispensed through a 0.22 μm sterile filter under a Class A environment into doses containing suitable volume and radioactivity at a calibration time.

The final composition may be formulated as a solution suitable for injection. The product is diluted to a standard radioactivity concentration, and therefore, the final volume of the bulk composition varies depending on the starting radioactivity of ¹⁷⁷Lu introduced. The solution meets the requirements for sterility and bacterial endotoxins according to the European pharmacopoeia confirming an acceptable manufacturing process from a microbial point of view.

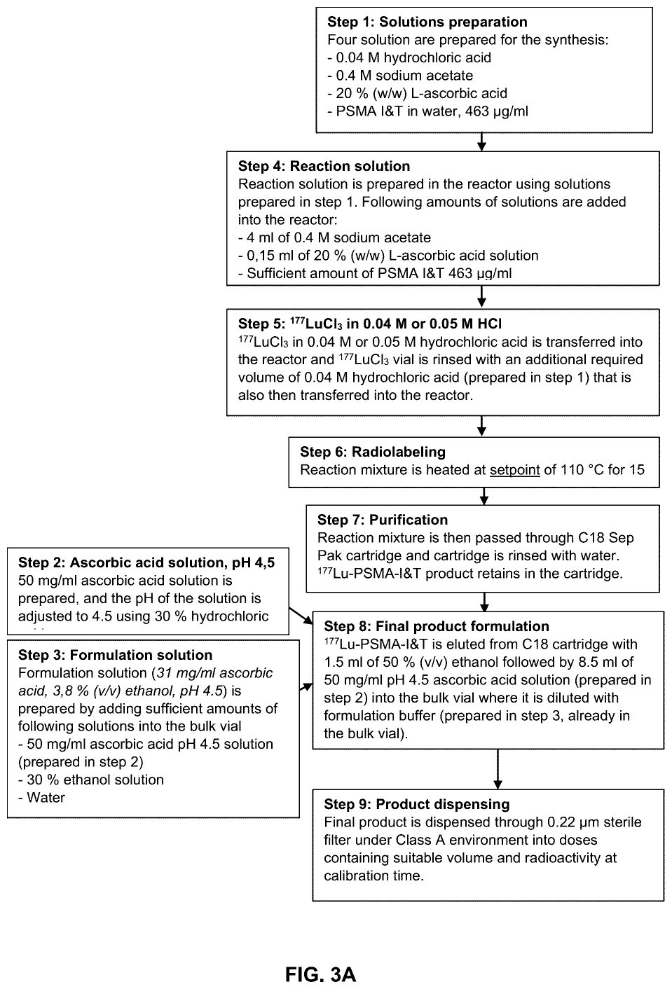

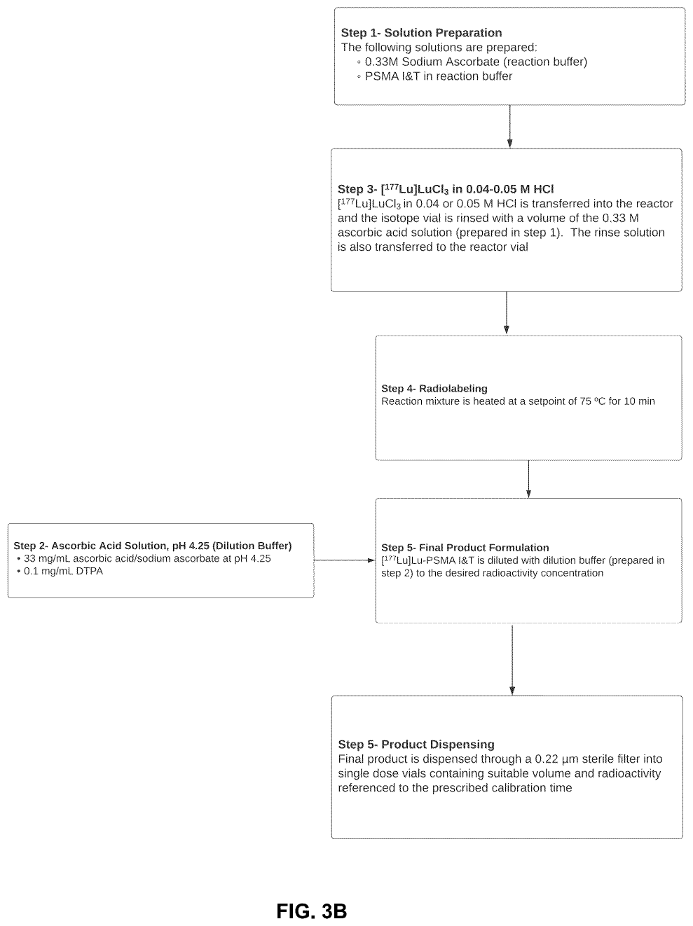

FIG. 3A provides one example of the process of making a radiopharmaceutical composition with purification of the reaction mixture and the formulation solution with ethanol. FIG. 3B provides one example of the process of making a radiopharmaceutical composition without purification and without ethanol.

Provided herein are methods of increasing the shelf life of a radiopharmaceutical product comprising ¹⁷⁷Lu-PSMA I&T. The method may include adjusting the pH of the composition to 3.5, 3.75, 4.0, 4.25, or 4.5, adjusting the amount of ascorbic acid in the composition, and/or adjusting the radioactivity to increase the shelf life of the composition by 0.25, 0.5, 0.75, 1, 1.25, 1.5, 2, 2.25, 2.5, 2.75, or 3 days. For example, the radiopharmaceutical composition may have a shelf life of 1, 1.5, 2, 2.25, 2.5, 2.75, 3, 3.25, 3.5, 3.75, 4, 4.25, 4.5, 4.75, or 5 days. In an embodiment, adjusting the pH, radioactivity, and/or ascorbic acid may increase the radiochemical purity of the composition to at least 99%, at least 98.5%, at least 98%, at least 97.5%, at least 97%, at least 96.5%, at least 96%, at least 95.5%, or at least 95% for up to 1, 1.5, 2, 2.25, 2.5, 2.75, 3, 3.25, 3.5, 3.75, 4, 4.25, 4.5, 4.75, or 5 days.

Target pharmaceutical formulations in accordance with the present disclosure are as provided in Table 1A.

TABLE 1A
Target pharmaceutical formulations

| | Target Pharmaceutical Formulation | | | |
|---|---|---|---|---|
| | Compositions | | | |
| Component | 1 and 2 | | Composition 3 | |
| Radioactivity concentration | ~1.0 GBq/mL | | 0.5-0.6 GBq/mL or | |
| | or 27 mCi/mL | | 13.5-16.2 mCi/mL | |
| Ascorbic acid | 42.5 | mg/mL | 31 | mg/mL |
| Disodium EDTA | 21 | μg/mL | 15.5 | μg/mL |
| Ethanol | 7.5% | (v/v %) | 3.8% | (v/v %) |
| pH | 5.5 | | 4.5 | |
| Shelf-life (tested up to 40° C.) | 48 | h | 72 | h |

### V. Stability

A stable non-radioactive labelled standard may be used for identifying the product peak in the HPLC analysis. The formulation may be prepared from an injection grade solution containing ascorbic acid, chelating agent (EDTA), absolute ethanol, and injections grade water. The formulation matrix may be prepared ex tempore as part of the synthesis preparation and predetermined amount is added to the bulk vial as part of the synthesis preparations.

Without being limited to any one theory, the radioactivity, the amount of ascorbic acid, and/or the pH of the solution may have an impact on the shelf life of the composition. Surprisingly, a lower concentration of ascorbic acid (e.g. 31 mg/ml vs. 42.5 mg/ml) in the composition, a pH 4.5 or lower, a low RAC, and/or the combinations thereof may result in a higher stability profile and a longer shelf life for the composition as compared to compositions with a pH 5 or higher, a high RAC, and/or combinations thereof. For example, this may be seen in FIG. 5. The shelf life may generally be determined based on the radiochemical purity of the composition after formulation or at the end of synthesis (EOS). The radiochemical purity may be confirmed by HPLC.

In one or more embodiments, ¹⁷⁷Lu-PSMA I&T formulation compositions with 31 mg/ml of ascorbic acid and pH of about 4.5 in a dose formulation at radioactivity concentration of 640 MBq/ml or below, can provide adequate stability of four days.

A composition having a low radioactivity concentration (e.g. 588.5 MBq/ml), a pH of 4.5, and 31 mg/ml ascorbic acid has 99.1% radiochemical purity 0 ours post EOS, 98.7% radiochemical purity 20 hours post EOS, 98.0% radiochemical purity 44 hours post EOS, 97.4% radiochemical purity 69 hours post EOS, and 97.0% radiochemical purity 93 hours post EOS. A composition having a low radioactivity concentration (e.g. 626 MBq/ml), a pH of 5.0, and 31 mg/ml ascorbic acid has 99.2% radiochemical purity 0 ours post EOS, 98.4% radiochemical purity 25 hours post EOS, 97.3% radiochemical purity 47 hours post EOS, and 96.5% radiochemical purity 71 hours post EOS. A composition having a low radioactivity concentration (e.g. 579 MBq/ml), a pH of 4.5, and 21 mg/ml ascorbic acid has 99.4% radiochemical purity 0 ours post EOS, 98.3% radiochemical purity 19 hours post EOS, 97.5% radiochemical purity 46 hours post EOS, 96.8% radiochemical purity 71 hours post EOS, and 96.0% radiochemical purity 92 hours post EOS. A composition having a high radioactivity concentration (e.g. 1,278 MBq/ml), a pH of 4.5, and 42.5 mg/ml ascorbic acid has 99.4% radiochemical purity 0 hours post EOS, 98.0% radiochemical purity 24 hours post EOS, 96.7% radiochemical purity 46 hours post EOS, 95.3% radiochemical purity 67 hours post EOS, and 95.2% radiochemical purity 71 hours post EOS.

The radiopharmaceutical composition may be stored at temperatures ranging from 2° C. to 40° C., about 2° C. to 5° C., about 5° C. to 10° C., about 10° C. to 15° C., about 15° C. to 20° C., about 20° C. to 25° C., about 25° C. to 30° C., about 30° C. to 35° C., or about 35° C. to 40° C.

In an embodiment, the radiopharmaceutical composition is stored at a temperature from about 5° C. to 40° C., about 10° C. to 35° C. or about 20° C. to 30° C. In one specific embodiment, the radiopharmaceutical composition is stored at a temperature at about 10° C., about 15° C., about 22° C., about 22.5° C., about 25° C., or at room temperature.

In one embodiment, the radiopharmaceutical composition is stored at about 22.5° C. In another embodiment, the radiopharmaceutical composition is stored at room temperature.

### VI. Specific Radiopharmaceutical Composition

In some embodiments, the medicinal product is a sterile filtered radiopharmaceutical solution containing a micro dose of ¹⁷⁷Lu-PSMA I&T solution in a 42.5 mg/ml aqueous ascorbic acid solution containing 7.5% (v/v) or 59 mg/n ethanol. The product is diluted to a standard radioactivity concentration and therefore the final volume of the bulk product varies depending on the starting radioactivity introduced. The composition of the final product described in Table 1B (¹⁷⁷Lu-PSMA I&T composition 1):

TABLE 1B
Composition of Final Product^{a} (¹⁷⁷Lu-PSMA I&T composition 1)

| Component | Quantity | | Function |
|---|---|---|---|
| ¹⁷⁷Lu-PSMA I&T | q.s.^{b} | | API |
| Ethanol | 7.5% (v/v) | | Vehicle/Stabilizing agent |
| | | | (radiolysis) |
| Ascorbic acid | 42.5 | mg | Stabilizing agent (radiolysis) |
| Disodium EDTA | 21 | μg | Metal ion chelator |

| | | |
|---|---|---|
| Sodium bicarbonate* | q.s. | pH adjuster |
| Sodium hydroxide* | q.s. | pH adjuster |
| WFI (injections grade | ad 1 ml | Vehicle |
| water) | | |

^{a}Max volume per vial is 10 ml
^{b}sufficient amount of radioactivity for intended use

In yet another embodiment, the medicinal product is a sterile filtered radiopharmaceutical solution containing a micro dose of ¹⁷⁷Lu-PSMA I&T solution in 31 mg/ml aqueous ascorbic acid solution containing 3.8% (v/v) or 30 mg/ml ethanol, at pH of about 4.5. The product is diluted to a standard radioactivity concentration, and therefore, the final volume of the bulk product varies depending on the starting radioactivity introduced. The composition is described below in Table 10C (¹⁷⁷Lu-PSMA I&T composition 2):

TABLE 1C
Composition of Final Product* (¹⁷⁷Lu-PSMA I&T composition 3)

| Component | Quantity | | Function |
|---|---|---|---|
| ¹⁷⁷Lu-PSMA I&T | q.s.** | | API |
| Ethanol | 3.8% (v/v) | | Vehicle/Stabilizing agent |
| | | | (radiolysis) |
| Ascorbic acid | 31 | mg/ml | Stabilizing agent (radiolysis) |
| Disodium EDTA | 15.5 | μg | Metal ion chelator |

| | | |
|---|---|---|
| Sodium bicarbonate | q.s | pH adjuster |
| Sodium hydroxide | q.s | pH adjuster |
| Hydrochloric acid | q.s | pH adjuster |
| WFI (injections grade | ad 1 ml | Vehicle |
| water) | | |

*Max volume per vial is 20 ml
**sufficient amount of radioactivity for intended use

### VII. Formulating the Drug Product

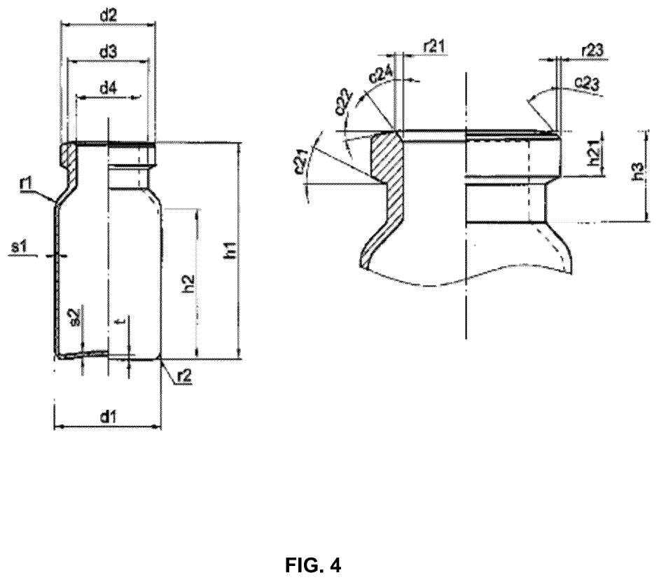

The drug product may be delivered in a sterile pyrogen free glass vial of Type 1 glass with a fluorocoated bromobutyl rubber septum. The septum is sealed with a crimped aluminum capsule. During transportation, the glass vial containing the radiopharmaceutical is kept in a lead shielded container. The transport container including lead shield and outer packaging complies to type A requirements (IAEA standards). FIG. 4 depicts a drawing of a product vial that can be used in this embodiment.

In one embodiment, the volume of the solution comprising the formulation or radiopharmaceutical composition is from about 10 ml to about 20 ml, from about 20 ml to about 30 ml, from about 30 ml to about 40 ml, from about 40 ml to about 50 ml, from about 50 ml to about 60 ml, from about 60 ml to about 70 ml, from about 70 ml to about 80 ml, from about 80 ml to about 90 ml, or from about 90 ml to about 100 ml. In one specific embodiment, the volume of the solution comprising the formulation or radiopharmaceutical composition is about 1 ml, about 5 ml, about 7 ml, about 8 ml, about 9 ml, about 10 ml, about 15 ml, about 20 ml, about 25 ml, or about 30 ml.

In one specific embodiment, the final volume in the dose vial is adjusted to between 7 ml and 10 ml or between 15 ml and 20 ml in order to provide the required amount of radioactivity at the date and time of infusion.

In another embodiment, ¹⁷⁷Lu-PSMA I&T injection is supplied as a single-dose vial or multi-dose vial. For example, provided herein is a radiopharmaceutical kit including a vial comprising a single dose of the ¹⁷⁷Lu-PSMA I&T injection product composition. In one embodiment, the strength of the ¹⁷⁷Lu-PSMA I&T injection product composition is about 0.1 GBq/ml, about 0.2 GBq/ml, about 0.3 GBq/ml, about 0.4 GBq/ml, about 0.5 GBq/ml, about 0.6 GBq/ml, about 0.7 GBq/ml, about 0.8 GBq/ml, about 0.9 GBq/ml, about 1.0 GBq/ml, about 1.1 GBq/ml, about 1.2 GBq/ml, about 1.3 GBq/ml, about 1.4 GBq/ml, about 1.5 GBq/ml, about 1.6 GBq/ml, about 1.7 GBq/ml, about 1.8 GBq/ml, about 1.9 GBq/ml, or about 2.0 GBq/ml. In another embodiment, the strength of the ¹⁷⁷Lu-PSMA I&T injection product composition is less than about 2.0 GBq/ml, less than about 1.5 GBq/ml, less than about 1.0 GBq/ml, or less than about 0.5 GBq/ml.

In yet another embodiment, the shelf life of the ¹⁷⁷Lu-PSMA I&T injection product composition is from about 30 hours to about 90 hours, from about 40 hours to about 80 hours, or from about 48 hours to about 72 hours. In one specific embodiment, the shelf life of the ¹⁷⁷Lu-PSMA I&T injection product composition is about 30 hours, about 35 hours, about 40 hours, about 45 hours, about 48 hours, about 50 hours, about 55 hours, about 60 hours, about 65 hours, about 70 hours, about 72 hours, about 75 hours, about 80 hours, about 85 hours, or about 90 hours.

In some embodiments, the radiopharmaceutical composition should have a radiochemical purity of 95% for ¹⁷⁷Lu-PSMA I&T to be sufficient for administration to a patient. The combined radiochemical impurities in the composition may be ≤5%. In various embodiments, the radiopharmaceutical composition may have a chemical purity such that Lu-PSMA I&T is present in the composition in a concentration of less than about 12 μg/ml, less than about 11 μg/ml, less than about 10 μg/ml, less than about 9 μg/ml, less than about 8 μg/ml, less than about 7 μg/ml, less than about 6 μg/ml, less than about 5 μg/ml, less than about 4 μg/ml, less than about 3 μg/ml, less than about 2 μg/ml, or less than about 1 μg/ml.

In some embodiments, the radiopharmaceutical composition may have an amount of colloidal ¹⁷⁷Lu of less than about 5% of radioactivity, less than about 4.5% of radioactivity, less than about 4% of radioactivity, less than about 3.5% of radioactivity, less than about 3% of radioactivity, less than about 2.5% of radioactivity, less than about 2% of radioactivity, less than about 1.5% of radioactivity, less than about 1% of radioactivity, less than about 0.5% of radioactivity, less than about 0.3% of radioactivity, less than about 0.2% of radioactivity, or less than about 0.1% of radioactivity In one embodiment, the radiopharmaceutical composition administered to the human patient in need thereof comprises less than 0.5%, less than 0.4%, less than 0.3%, less than 0.2%, or less than 0.1% colloidal ¹⁷⁷Lu.

In some embodiments, the radiopharmaceutical composition may have less than about 17.5 EU/ml, less than about 17 EU/ml, less than about 16.5 EU/ml, less than about 16 EU/ml, less than about 15.5 EU/ml, less than about 15 EU/ml, less than about 14.5 EU/ml, less than about 14 EU/ml less than about 13.5 EU/ml, less than about 13 EU/ml, less than about 12.5 EU/ml, less than about 12 EU/ml, less than about 11.5 EU/ml, less than about 11 EU/ml, less than about 10.5 EU/ml, less than about 10 EU/ml, less than about 9.5 EU/ml, less than about 9 EU/ml, less than about 8.5 EU/ml, less than about 8 EU/ml, less than about 7.5 EU/ml, less than about 7 EU/ml, less than about 6.5 EU/ml, less than about 6 EU/ml, less than about 5.5 EU/ml, less than about 5 EU/ml, less than about 4.5 EU/ml, less than about 4 EU/ml, less than about 3.5 EU/ml, less than about 3 EU/ml, less than about 2.5 EU/ml, less than about 2 EU/ml, less than about 1.5 EU/ml, less than about 1 EU/ml, less than about 0.5 EU/ml, or no bacterial endotoxins.

In an embodiment, the radiochemical purity of the composition is 95% at 1 day, up to 2 days, up to 3 days, up to 4 days, or up to 5 days after formulation. In additional embodiments, the radiochemical purity of the composition is 95% at 24 hours, up to 36 hours, up to 48 hours, up to 72 hours, or up to 96 hours after formulation. In further embodiments, the radiochemical purity of the composition is suitable for injection and suitable for administration to a patient in need thereof more than 72 hours after formulation, more than 96 hours after formulation, or more than 100 hours after formulation. The radiopharmaceutical composition may have a radiochemical purity of at least 95%, at least 95.5%, at least 96%, at least 96.5%, at least 97%, at least 97.5%, at least 98%, at least 98.5%, or at least 99% at 24 hours, 48 hours, 72 hours, and/or 96 hours after formulation. In some examples, the radiopharmaceutical composition may have a radiochemical purity of 95.0% or greater, 95.5% or greater, 96.0% or greater, 96.5% or greater, 97.0% or greater, 97.5% or greater, 98.0% or greater, 98.5% or greater, 99.0% or greater, or 99.5% or greater at administration. For example, the radiopharmaceutical composition may have a radiochemical purity of more than 95% at 46 to 48 hours after formulation, a radiochemical purity of more than 96% at 46 to 48 hours after formulation, a radiochemical purity of more than 97% at 46 to 48 hours after formulation, a radiochemical purity of more than 95% at 69 to 72 hours after formulation, a radiochemical purity of more than 96% at 69 to 72 hours after formulation, a radiochemical purity of more than 97% at 69 to 72 hours after formulation, a radiochemical purity of more than 95% at 90 to 93 hours after formulation, a radiochemical purity of more than 96% at 90 to 93 hours after formulation, and/or a radiochemical purity of more than 97% at 90 to 93 hours after formulation.

In some examples, the radiochemical purity of the composition may range from about 99.0% to about 99.4% 0 hours post EOS. In various embodiments, the radiochemical purity of the composition may range from about 96.5% to about 98.7% 19-25 hours post EOS. In other examples, the radiochemical purity of the composition may range from about 93.3% to about 98.0% 44-47 hours post EOS. In additional examples, the radiochemical purity of the composition may range from about 91.2% to about 97.4% 69-71 hours post EOS. In some examples, the radiochemical purity of the composition may range from about 94.5% to about 97.0% 90-93 hours post EOS.

In another embodiment, ¹⁷⁷Lu-PSMA I&T injection is supplied as a single-dose vial or multi-dose vial.

In yet another embodiment, a patient in need of radioligand therapy during a treatment receives a single intravenous radioactive dose at the beginning of a treatment cycle. The treatment cycle is from 1 to 10 weeks. In one embodiment, the treatment includes 1 to 6 treatment cycles. In another embodiment, a dose reduction or a dose increase is introduced during treatment.

In one embodiment, the volume of the patient dose is calculated depending on the radioactive dose to be administered.

In another embodiment, ¹⁷⁷Lu-PSMA I&T is injected by intravenous (IV) route slowly, over about 10 minutes, and followed by infusion of 500-1000 mL of Ringer's or normal saline solution. An extra 7 mL injection when the total blood volume is over 5,000 mL is of no consequence. The dose is administered every 6 weeks for 4 cycles.

In yet another embodiment, a patient in need of radioligand therapy during a treatment receives a single intravenous radioactive dose at the beginning of a treatment cycle. The treatment cycle is from 1 to 10 weeks. In one embodiment, the treatment includes 1-6 treatment cycles. In another embodiment, a dose reduction or a dose increase is introduced during treatment.

In one embodiment, the volume of the patient dose is calculated depending on the radioactive dose to be administered.

### VIII. Method of Diagnosing or Treatment of Prostate Cancer

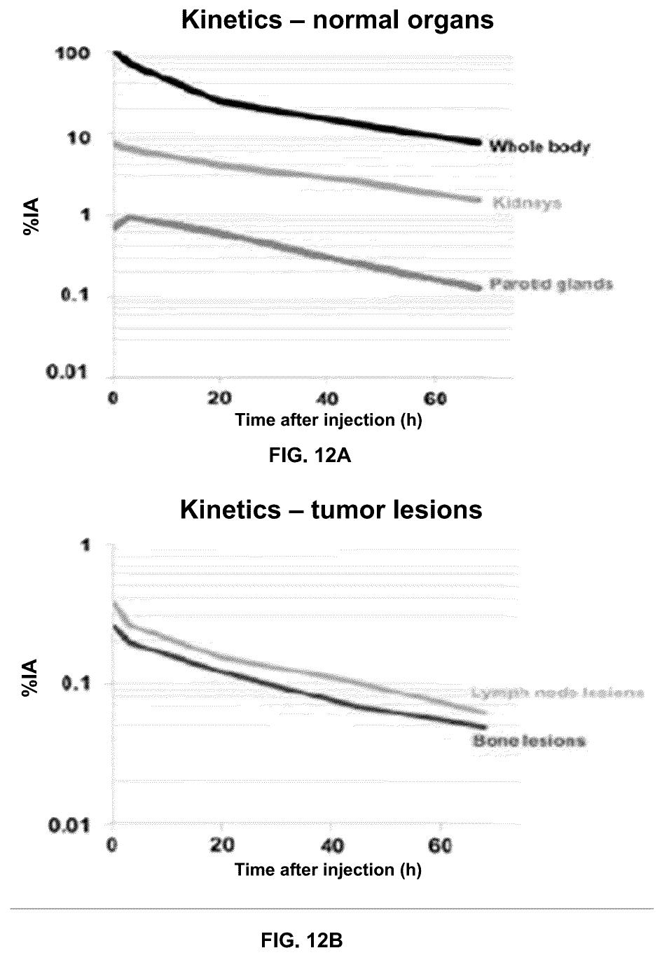

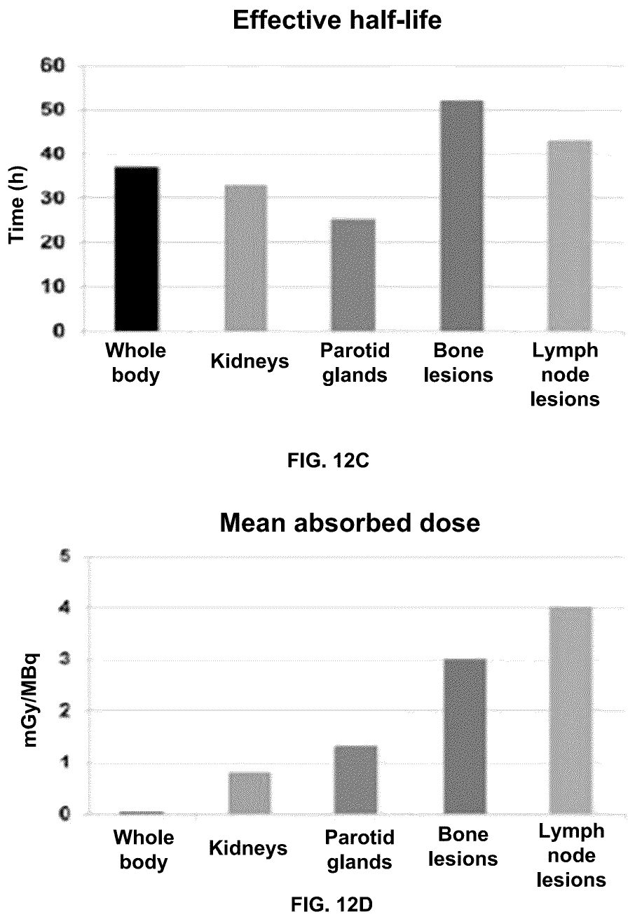

Provided herein are methods of diagnosing or treating a tumor of a patient in need thereof. The method may include administering by injection a radiopharmaceutical composition comprising 177Lu-PSMA I&T and ascorbic acid at a pH of 3.5 to 4.5 in solution. After administration, the radiopharmaceutical composition may have one or more of the kinetic properties shown in FIGS. 12A-12D.

FIG. 12A shows the kinetics of the radiopharmaceutical composition in normal organs, including the whole body, kidneys, and parotid glands over 60 hours. In an embodiment, less than 20 hours after injection, the radiopharmaceutical composition may have an activity of about 30% IA to 100% IA, 30% IA to 40% IA, 40% IA to 50% IA, 50% IA to 60%, 60% IA to 70% IA, 70% IA to 80% IA, 80% IA to 90% IA, or 90% IA to 100% IA. IA In some embodiments, 20 hours after injection the radiopharmaceutical composition may have an activity of at least 20% IA to 30% IA, 20% IA to 21% IA, 21% IA to 22% IA, 22% IA to 23% IA, 23% IA to 24% IA, 24% IA to 25% IA, 25% IA to 26% IA, 26% IA to 27% IA, 27% IA to 28% IA, 28% IA to 29% IA, or 29% IA to 30% IA in the whole body. In some embodiments, 40 hours after injection the radiopharmaceutical composition may have an activity of at least 10% IA to 20% IA, 11% IA to 11% IA, 11% IA to 12% IA, 12% IA to 13% IA, 13% IA to 14% IA, 14% IA to 15% IA, 15% IA to 16% IA, 16% IA to 17% IA, 17% IA to 18% IA, 18% IA to 19% IA, 11% IA to 12% IA in the whole body. In some embodiments, 60 hours after injection the radiopharmaceutical composition may have an activity of at least 5% IA to 10% IA, 5% IA to 6% IA, 6% IA to 7% IA, 7% IA to 8% IA, 8% IA to 9% IA, or 9% IA to 10% IA in the whole body.

In an embodiment, less than 20 hours after injection the radiopharmaceutical composition may have an activity of at least 8% IA to 10% IA, 8% IA to 8.5% IA, 8.5% IA to 9% IA, 9% IA to 9.5% IA, or 9.5% IA to 10% IA in the kidneys. In some embodiments, 20 hours after injection the radiopharmaceutical composition may have an activity of at least 3% IA to 8% IA, 3% IA to 4% IA, 4% IA to 5% IA, 5% IA to 6% IA, 6% IA to 7% IA, or 7% IA to 8% IA in the kidneys. In some embodiments, 40 hours after injection the radiopharmaceutical composition may have an activity of at least 1% IA to 5% IA, 1% IA to 2% IA, 2% IA to 3% IA, 3% IA to 4% IA, or 4% IA to 5% IA in the kidneys. In some embodiments, 60 hours after injection the radiopharmaceutical composition may have an activity of at least 1% IA to 5% IA, 1% IA to 2% IA, 2% IA to 3% IA, 3% IA to 4% IA, or 4% IA to 5% IA in the kidneys.

In another embodiment, less than 20 hours after injection the radiopharmaceutical composition may have an activity of at least 0.7% IA to 1.0% IA, 0.7% IA to 0.8% IA, 0.8% IA to 0.9% IA, or 0.9% IA to 1.0% IA in the parotid glands. In some embodiments, 20 hours after injection the radiopharmaceutical composition may have an activity of at least 0.3% IA to 0.8% IA, 0.3% IA to 0.4% IA, 0.4% IA to 0.5% IA, 0.5% IA to 0.6% IA, 0.6% IA to 0.7% IA, or 0.7% IA to 0.8% IA in the parotid glands. In some embodiments, 40 hours after injection the radiopharmaceutical composition has an activity of at least 0.2% IA to 0.5% IA, 0.2% IA to 0.3% IA, 0.3% IA to 0.4% IA, or 0.4% IA to 0.5% IA in the parotid glands. In some embodiments, 60 hours after injection the radiopharmaceutical composition has an activity of at least 0.1% IA to 0.3% IA, 0.1% IA to 0.15% IA, 0.15% IA to 0.2% IA, 0.2% IA to 0.25% IA, 0.25% IA to 0.3% IA in the parotid glands.

FIG. 12B shows the kinetics of the radiopharmaceutical composition in tumor lesions, including lymph node lesions and bone lesions over 60 hours. In further embodiments, less than 20 hours after injection the radiopharmaceutical composition may have an activity of at least 0.2% IA to 0.5% IA, 0.2% IA to 0.3% IA, 0.3% IA to 0.4% IA, or 0.4% IA to 0.5% IA in lymph node lesions of the patient. In some embodiments, 20 hours after injection the radiopharmaceutical composition may have an activity of at least 0.1% IA to 0.3% IA, 0.1% IA to 0.15% IA, 0.15% IA to 0.2% IA, 0.2% IA to 0.25% IA, 0.25% IA to 0.3% IA in the lymph node lesions. In some embodiments, 40 hours after injection the radiopharmaceutical composition has an activity of at least 0.08% IA to 0.2% IA, 0.08% IA to 0.1% IA, 0.1% IA to 0.12% IA, 0.12% IA to 0.14% IA, 0.14% IA to 0.16% IA, 0.16% IA to 0.18% IA, 0.18% IA to 0.2% IA in the lymph node lesions. In some embodiments, 60 hours after injection the radiopharmaceutical composition has an activity of at least 0.05% IA to 0.1% IA, 0.06% IA to 0.06% IA, 0.06% IA to 0.07% IA, 0.07% IA to 0.08% IA, 0.08% IA to 0.09% IA, or 0.09% IA to 0.1% IA in the lymph node lesions.

In yet further embodiments, less than 20 hours after injection the radiopharmaceutical composition may have an activity of at least 0.1% IA to 0.4% IA, 0.1% IA to 0.2% IA, 0.2% IA to 0.3% IA, or 0.3% IA to 0.4% IA in bone lesions of the patient. In some embodiments, 20 hours after injection the radiopharmaceutical composition may have an activity of at least 0.1% IA to 0.2% IA, 0.1% IA to 0.12% IA, 0.12% IA to 0.14% IA, 0.14% IA to 0.16% IA, 0.16% IA to 0.18% IA, 0.18% IA to 0.2% IA in the bone lesions. In some embodiments, 40 hours after injection the radiopharmaceutical composition may have an activity of at least 0.05% IA to 0.1% IA, 0.06% IA to 0.06% IA, 0.06% IA to 0.07% IA, 0.07% IA to 0.08% IA, 0.08% IA to 0.09% IA, or 0.09% IA to 0.1% IA in the bone lesions. In some embodiments, 60 hours after injection the radiopharmaceutical composition has an activity of at least 0.02% IA to 0.05% IA, 0.02% IA to 0.03% IA, 0.03% IA to 0.04% IA, or 0.04% IA to 0.05% IA in the bone lesions.

FIG. 12C shows the effective half-life of the radiopharmaceutical composition in both normal organs and tumor lesions. In an embodiment, the radiopharmaceutical composition may have an effective half-life of about 30 hours to 40 hours, 30 hours to 32 hours, 32 hours to 34 hours, 34 hours to 36 hours, 36 hours to 38 hours, or 38 hours to 40 hours in the whole body of the patient. In some embodiments, the effective half-life of the radiopharmaceutical composition may be about 25 hours to 35 hours, 25 hours to 27 hours, 27 hours to 29 hours, 29 hours to 31 hours, 31 hours to 33 hours, or 33 hours to 35 hours in the kidneys of the patient. In some embodiments, the effective half-life of the radiopharmaceutical composition may be about 20 hours to 30 hours, 20 hours to 22 hours, 22 hours to 24 hours, 24 hours to 26 hours, 26 hours to 28 hours, or 28 hours to 30 hours in the parotid glands of the patient. In some embodiments, the effective half-life of the radiopharmaceutical composition may be about 45 hours to 55 hours, 45 hours to 47 hours, 47 hours to 49 hours, 49 hours to 51 hours, 51 hours to 53 hours, or 53 hours to 55 hours in bone lesions of the patient. In some embodiments, the effective half-life of the radiopharmaceutical composition may be about 35 hours to 45 hours, 35 hours to 37 hours, 37 hours to 39 hours, 39 hours to 41 hours, 41 hours to 43 hours, or 43 hours to 45 hours in lymph node lesions of the patient.

FIG. 12D shows the mean absorbed dose of the radiopharmaceutical composition in both normal organs and tumor lesions. In an embodiment, a mean absorbed dose of the radiopharmaceutical is about 0.01 mGy/MBq to 0.5 mGy/MBq, 0.01 mGy/MBq to 0.05 mGy/MBq, 0.05 mGy/MBq to 0.1 mGy/MBq, 0.1 mGy/MBq to 0.2 mGy/MBq, 0.2 mGy/MBq to 0.3 mGy/MBq, 0.3 mGy/MBq to 0.4 mGy/MBq, or 0.4 mGy/MBq to 0.5 mGy/MBq in the whole body of the patient. In some embodiments, the mean absorbed dose of the radiopharmaceutical composition may be about 0.5 mGy/MBq to 1.0 mGy/MBq, 0.5 mGy/MBq to 0.6 mGy/MBq, 0.6 mGy/MBq to 0.7 mGy/MBq, 0.7 mGy/MBq to 0.8 mGy/MBq, 0.8 mGy/MBq to 0.9 mGy/MBq, 0.9 mGy/MBq to 1.0 mGy/MBq in the kidneys of the patient. In some embodiments, the mean absorbed dose of the radiopharmaceutical composition may be about 1 mGy/MBq to 1.5 mGy/MBq, 1.0 mGy/MBq to 1.1 mGy/MBq, 1.1 mGy/MBq to 1.2 mGy/MBq, 1.2 mGy/MBq to 1.3 mGy/MBq, 1.3 mGy/MBq to 1.4 mGy/MBq, or 1.4 mGy/MBq to 1.5 mGy/MBq in the parotid glands of the patient. In some embodiments, the mean absorbed dose of the radiopharmaceutical composition may be about 2.5 mGy/MBq to 3.5 mGy/MBq, 2.5 mGy/MBq to 2.7 mGy/MBq, 2.7 mGy/MBq to 2.9 mGy/MBq, 2.9 mGy/MBq to 3.1 mGy/MBq, 3.1 mGy/MBq to 3.3 mGy/MBq, or 3.3 mGy/MBq to 3.5 mGy/MBq in bone lesions of the patient. In some embodiments, the mean absorbed dose of the radiopharmaceutical composition may be about 3.5 mGy/MBq to 4.5 mGy/MBq, 3.5 mGy/MBq to 3.7 mGy/MBq, 3.7 mGy/MBq to 3.9 mGy/MBq, 3.9 mGy/MBq to 4.1 mGy/MBq, 4.1 mGy/MBq to 4.3 mGy/MBq, or 4.3 mGy/MBq to 4.5 mGy/MBq in lymph node lesions of the patient.

Treatment aimed at eradicating the primary tumor, typically with surgery or radiation, is unsuccessful in ˜30% of men, who develop recurrent disease that usually manifests first as a rise in plasma prostate-specific antigen (PSA) followed by metastasis to distant sites (Stephenson et al. J Clin Oncol, 2005; 23:8253-61). Given that prostate cancer cells depend on androgen receptors (AR) for their proliferation and survival, the standard treatment for patients with recurrent disease is androgen deprivation therapy (ADT) with a gonadotropin releasing hormone analog (GnRHa)) with or without an anti-androgen.

Treatment results with ADT are generally predictable: a decline in PSA followed by tumor regression, a period of stability in which the tumor does not proliferate and stable PSA, followed by rising PSA and regrowth defined as a castration-resistant disease. Nearly all men with progressive prostate cancer eventually develop castration-resistant disease. Prostate cancer progression despite castrate levels of testosterone represents a transition to a lethal disease stage. Docetaxel with prednisone, cabazitaxel with prednisone, enzalutamide, and abiraterone with prednisone have become the Standard of Care based on the National Comprehensive Cancer Network (NCCN) guidelines (Mohler et al., NCCN Clinical practice guidelines in oncology. Prostate Cancer, (Version 2.2019). JNCCN.org; 17(5), 479-505) in men with metastatic castration-resistant prostate cancer (mCRPC).

Abiraterone, enzalutamide, and docetaxel with prednisone are all indicated for patients with mCRPC as first line of treatment, while cabazitaxel with prednisone is indicated only for mCRPC patients who progressed on docetaxel. George et al. reported the sequencing of treatments for mCRPC patients in a real-world clinical setting in the United States (George et al. 2020). A higher proportion of patients in the US receive androgen receptor axis-targeted therapy (ARAT, namely abiraterone and enzalutamide) as a first line treatment versus docetaxel; similarly, a higher proportion of mCRPC patients receive the alternative ARAT as second line of treatment (enzalutamide after abiraterone, or vice versa).

Targeted radionuclide therapy has become an attractive and emerging therapy option for many different cancers, including lymphoma, melanoma, and neuroendocrine tumors (Kraeber-Bodéré et al., Semin Oncol, 2014, 41, 613-22; Mier et al., J Nucl Med, 2014, 55, 9-14; Bodei et al., Eur J Nucl Med Mol Imaging, 2015, 42, 5-19). Prostate-specific membrane antigen (PSMA) is a key target for radionuclide diagnosis and therapy for PC. PSMA is normally expressed in prostate cells as well as some extraprostatic tissue but its overexpression in prostate cancer cells makes it an attractive target for therapeutic agents with the potential to limit systemic toxicity (Silver et al, Clin Cancer Res. 1997 January; 3(1):81-5. PMID: 9815541). Initial clinical experience with PSMA-based radionuclide treatment of PC using ¹³¹I-labeled PSMA showed promising results with a PSA decrease >50% in 60% of all treated prostate cancer patients and with mild hematotoxicity (Zechmann et al., Eur J Nucl Med Mol Imaging, 2014, 41, 1280-92).

### IX. Administration

Further provided herein are methods of administering the radiopharmaceutical composition. The radiopharmaceutical composition may be administered by injection to a human patient in need thereof.

There can be about six main aspects of administration.

First, cooling the salivary glands, the patients receive ice packs over the parotid and submandibular glands from 30 min prior to and up to 4 hours after administration of ¹⁷⁷Lu-PSMA I&T to reduce the risk of salivary gland radiation injuries. There is no scientific evidence of whether cooling the salivary glands is an effective therapy for saving these glands from radiation; however, it is tolerable and not harmful for the patients.

Second, using a urinary catheter in incontinent patients in the first 48 hours to avoid any contamination.

Third, activity of 6.5-7.5 GBq (range: 6.0-8.0 GBq)¹⁷⁷Lu-PSMA I&T. The amount of activity can be reduced to 4.0-5.0 GBq in the case of impaired renal function (e.g., Creatinine within 1.0-1.5 UNL). According to the preliminary results, an activity of 7.4 GBq can be administered safely; however, more data are required to increase the amount of activity.

Fourth, infusion of the activity intravenously as a slow bolus (over about 1-15 minutes) followed by 500-1000 ml Ringer or NaCl solution. The patients should be encouraged to void as frequently as possible and drink about 2 liters of water daily. In patients with dilated non-obstructive renal disease an administration of diuretics may be meaningful.

Fifth, in average 3-5 cycles of the RLT every 5-8 weeks, experience up to 11 cycles has been reported. In the case of continuously increasing PSA, after the first two cycles accompanied by worsening of the general condition, the indication of further RLTs should be re-evaluated. In case of a decreasing PSA to <1.0 μg/l during the therapy cycles, a PSMA imaging could evaluate existence of small PSMA-positive metastases after completion of RLT when post injection SPECT study is not enough informative. In case of a significant decline of platelets or leukocytes, the time interval between the 2 cycles can be prolonged.

Sixth, at least one whole body scan 24-48 hours post injection (preferably with SPECT(/CT). In patients with diffuse bone and bone marrow metastases as well as in patients with brain metastases a concomitant corticosteroid therapy (e.g., prednisolone 20 mg/daily) in the first two weeks after administration is advisable.

In some embodiments, the method may include injecting the radiopharmaceutical composition into a patient in need thereof more than 48 hours after formulation. In some examples, the radiopharmaceutical composition may include ¹⁷⁷Lu-PSMA I&T and ascorbic acid in a solution having a pH of 3.5 to 4.5, and the solution may have a radiochemical purity of more than 96% when administered. In an embodiment, the pH of the solution is about 3.5 to 4.2. The composition may include <6 μg/ml of Lu-PSMA I&T, about 7 μg/ml to about 18 μg/ml disodium EDTA, about 25 μl/ml to about 45 μl/ml ethanol, and/or about 15 to about 35 mg/ml ascorbic acid. The composition may have a radioactivity of about 0.5 GBq/ml or about 13.5 mCi/ml and have a radiochemical purity of at least 98% at 44 hours after formulation, at least 97% at 69 hours after formulation, and/or at least 97% at 93 hours after formulation.

The pharmaceutical composition may be administered as 2-11 cycles/treatments every 5-8 weeks. In some embodiments, the patient may be administered up to 1, 2, 3, 4, 5, 6, 7, 8, 9, 10, or 11 treatments and the treatments may be administered every 4, 5, 6, 7, or 8 weeks. In an example, the patient may be administered up to 4 treatments, with each treatment administered every 6 weeks.

In various embodiments, the patient may be administered a dose of 0.5 GBq to 10 GBq. For example, the radiopharmaceutical composition may contain a standard radioactivity of about 200 mCi at the time of expiry with a standard radioactivity concentration of about 27 mCi/mL at end of production; therefore, the final volume of the dose vial may be adjusted to between 7 and 10 mL in order to provide the required amount of radioactivity at the date and time of infusion. In at least one example, the patient may be administered a dose of about 200 mCi (7.4 GBq±0.1 GBq) for each treatment. In one aspect, the patient may be administered a dose of about 200 mCi (7.4 GBq±0.1 GBq) for each treatment for four, five, six, or more treatments. In another aspect, the patient may be administered a dose of about 200 mCi (7.4 GBq±0.1 GBq) for each treatment for four or more treatments, five or more, six or more, seven or more, or eight or more treatments. In yet another aspect, the patient may be administered a dose of about 200 mCi (7.1 GBq) for each treatment for four, five, six, seven, eight, or more treatments.

Upon administration of the radiopharmaceutical composition to a patient, the patient may maintain low levels of hematotoxic and nephrotoxic toxicity. In some embodiments, the prostate-specific antigen (PSA) decline is more than about 40%, more than about 45%, more than about 50%, more than about 55%, more than about 60%, more than about 65%, more than about 70%, more than about 75%, or more than about 80%.

Further provided herein are methods of treating a patient with mCRPC by administering the radiopharmaceutical composition comprising ¹⁷⁷Lu-PSMA I&T. The method may further comprise imaging the patient using PSMA-PET to document and confirm the patient is mCRPC positive prior to administering the radiopharmaceutical composition. For example, the patient may have a PSMA-PET scan (e.g. [⁶⁸Ga]Ga-PSMA-11 or [¹⁸F]DCFPyL) positive as determined by central reader.

### Indications and Contraindications

RLT with ¹⁷⁷Lu-PSMA I&T may be indicated for the treatment of patients with mCRPC, who do not have any other approved therapy option planned by a multidisciplinary team.

In some embodiments, the patient also has histologically or pathologically confirmed prostate adenocarcinoma without predominant small cell component, has progressive disease by one or more of the following criteria: a) Serum/plasma PSA progression defined as 2 consecutive increases in PSA over a previous reference value measured at least 1 week apart with a minimum start value of >2 ng/mL; or b) Progression of measurable disease (RECIST 1.1) or presence of at least two new bone lesions (PCWG3 criteria), and/or has had previous treatment with a next-generation androgen receptor (AR)-directed therapy (e.g. abiraterone, enzalutamide, apalutamide, darolutamide). In additional embodiments, the patient may have effective castration with a serum testosterone level of <50 ng/dL and plan to continue with chronic medical or surgical castration. mCRPC patients should undergo hormone therapy and chemotherapy as well as bone targeted therapy, if indicated.

In at least one example, a patient in need of RLT using ¹⁷⁷Lu-PSMA I&T may fulfill the following criteria:
1) mCRPC with PSMA positive metastatic disease based upon PSMA-PET or SPECT imaging. There are no limitations regarding the number or site of metastases, i.e. bone or soft tissue metastases. Caution should be given to patients e.g. with diffuse bone marrow, perineural and brain metastases.
2) After initial hormone therapy (LH-RH agonists/antagonists). Progressive disease, i.e. biochemical and/or radiologic progression, despite newly developed hormone therapies (Abiraterone/Enzalutamide) or these medications may be avoided by the patient. Progressive disease despite chemotherapy (Docetaxel and Cabazitaxel) or the patient being unfit for chemotherapy or avoiding chemotherapy.
3) Not suitable for ¹⁵³Sm-EDTMP or [²²³Ra]RaCl₂ or other local available radiopharmaceuticals for bone-targeted therapies due to extra-osseous metastases or diffuse bone marrow metastases or avoided by the patient. In patients without adequate response to bone-targeted therapies for pain palliation or exacerbation of pain even by such therapy, an RLT with ¹⁷⁷Lu-PSMA I&T can be evaluated.
4) Life expectancy longer than 4-6 months.
5) Decision for salvage therapy at the institutional interdisciplinary tumor board.

In summary, mCRPC patients should undergo hormone therapy and chemotherapy as well as bone targeted therapy, if indicated. In the case of any contraindication for one of these therapies, it should be discussed and documented in an interdisciplinary tumor board.

Contraindications are as follows:
(1) WBC ≤1×10⁹/l.
(2) Hb ≤80 g/l. (In the case of symptomatic anemia, a red blood cell transfusion should precede the therapy. RLT with ¹⁷⁷Lu-PSMA I&T may have a positive effect on bone marrow depression, causing the need for less blood to be transfused due to tumor regression in bone marrow. It should be noted that pure anemia without thrombocytopenia and leucopenia is not a contraindication for RLT.)
(3) Platelets ≤70×10⁹/l.
(4) Creatinine >1.5 UNL; Kidney failure with creatinine clearance <30 mL/min
(5) Absolute obstruction in renal excretion.
(6) Previous chemotherapy or bone-targeted radionuclide therapy and extended external beam irradiation fields to the bone marrow (pelvis, spine), if performed during 4 weeks preceding the RLT
(7) ECOG performance status >2.
(8) Hypersensitivity to the active substance or to any of the excipients.

After a patient in need thereof has been identified, the activity of the radiopharmaceutical composition may be confirmed prior to administration. The radioactivity of the ¹⁷⁷Lu-PSMA I&T composition may be 6.5-7.5 GBq or within a range of 6.0-8.0 GBq. The radioactivity may be reduced to 4.0-5.0 GBq in the case of impaired renal function (e.g. Creatinine within 1.0-1.5 UNL).

The radiopharmaceutical composition solution may be infused intravenously as a slow bolus (over about 10-15 minutes) followed by 500-1000 ml Ringer or NaCl solution. The patient may be encouraged to void as frequently as possible and drink about 2 liters of water daily. Patients with dilated non-obstructive renal disease may be administered diuretics.

The pharmaceutical composition may be administered as 2-11 cycles of the RLT every 5-8 weeks. In the case of continuously increasing PSA, after the first two cycles accompanied by worsening of the general condition, the indication of further RLTs may be re-evaluated. In the case of a decreasing PSA to <1.0 μg/l during the therapy cycles, a PSMA imaging may evaluate existence of small PSMA-positive metastases after completion of RLT when a post injection SPECT study is not enough to be informative. In the case of a significant decline of platelets or leukocytes, the time interval between 2 cycles may be prolonged.

At least one whole body scan 24-48 hours post injection (preferably with SPECT/CT) may be performed. In patients with diffuse bone and bone marrow metastases as well as in patients with brain metastases, a concomitant corticosteroid therapy (e.g. prednisolone 20 mg/daily) may be administered in the first two weeks after administration of the radiopharmaceutical composition.

In some embodiments, after administration of the radiopharmaceutical composition, the patient may have improved radiographic progression free survival (rPFS). A patient administered the radiopharmaceutical composition may have an rPFS of about 6 to about 12 months after initiating administration of the radiopharmaceutical composition. In various embodiments, the patient administered the radiopharmaceutical composition may have an rPFS of at least 6 months, 7 months, 8 months, 9 months, 10 months, 11 months, or 12 months after initiating administration of the radiopharmaceutical composition. For example, treatment of patients with ¹⁷⁷Lu-PSMA I&T may increase the rPFS from 6 months using standard of care to up to 10 months using the radiopharmaceutical composition. Radiographic progression free survival may be defined as the time from randomization to radiographic progression (using PCWG3 and RECIST 1.1 criteria as assessed by blinded independent central review [BICR]) or death due to any cause.

In an embodiment, the patient may have improved overall survival (OS) after initiating administration of the radiopharmaceutical composition. A patient administered the radiopharmaceutical composition may have an overall survival of about 18 to about 26 months after initiating administration of the radiopharmaceutical composition. In various embodiments, the patient administered the radiopharmaceutical composition may have an OS of at least 18 months, 19 months, 20 months, 21 months, 22 months, 23 months, 24 months, 25 months, or 26 months after initiating administration of the radiopharmaceutical composition. For example, treatment of patients with ¹⁷⁷Lu-PSMA I&T may increase the OS from 18 months using standard of care to up to 25 months using the radiopharmaceutical composition.

In another embodiment, the patient may have improved second radiographic progression free survival (rPFS 2) after initiating administration of the radiopharmaceutical composition.

In some embodiments, the patient may have improved progression free survival after initiating administration of the radiopharmaceutical composition. In additional embodiments, the patient may have improved second progression-free survival after initiating administration of the radiopharmaceutical composition. The second progression-free survival may be the second occurrence of PCWG3 progression, clinical/symptomatic progression and/or pain progression, or death due to any cause.

In an embodiment, the patient may have an improved PSA₅₀ response rate after initiating administration of the radiopharmaceutical composition. The PSA₅₀ response rate may be the response rate of patients who achieve a reduction of 50% in PSA from the baseline PSA assessment.

In an embodiment, the patient may have an improved time to first symptomatic skeletal event (SSE) after initiating administration of the radiopharmaceutical composition. An SSE may be the occurrence of either bone-directed radiotherapy to relieve bone pain, new symptomatic pathologic fractures, spinal cord compression, or tumor-related orthopedic surgery.

In an embodiment, the patient may have an improved time to soft tissue progression (STP) after initiating administration of the radiopharmaceutical composition. STP may include the occurrence of radiographic progression in soft tissue. In another embodiment, the patient may have an improved time to chemotherapy (TTC) after initiating administration of the radiopharmaceutical composition.

In an embodiment, the patient may have improved results on a Quality of Life Questionnaire after initiating administration of the radiopharmaceutical composition. For example, the Quality of Life (QoL) may be assessed via European Organisation for Research and Treatment of Cancer Quality of Life Questionnaire C30 (EORTC QLQ-C30). The EORTC QLQ-C30 is a questionnaire of thirty quality of life (QoL) questions developed to assess the QoL of cancer patients. The EORTC QLQ-C30 comprises 30 items, 24 of which are aggregated into nine multi-item scales, which are scored from 0 to 100.

### Examples

The following non-limiting examples are provided for illustrative purposes only, and therefore should not be viewed in a limiting sense.

### Analytical Procedures

The product was identified by subsequent injection of reference solution of Lu-PSMA I&T and formulated solution to a liquid chromatography system. Radio nuclidic identity was determined by gamma ray energy detection.

pH was estimated by pH paper. Radioactivity was measured in a dose calibrator. Radiochemical purity was determined by liquid chromatography with radioactivity detection and thin layer chromatography.

The radioactivity of ¹⁷⁷Lu-PSMA I&T was determined by a dose calibrator when the dose is dispensed.

Bacterial endotoxin content was determined for each batch before release using a PTS-tester (Ph Eur method D). Sterility was determined according to Ph Eur.

Qualities of analytical procedures used for the drug product—e.g., specificity, linearity, and reproducibility—were investigated by using a known reference standard for the unlabeled precursor. All analytical procedures were found suitable for their intended use.

An acceptance criterion for the radioactivity in the formulation is not set since this will vary depending on the individual clinical need assessed by the health care professional responsible for administering the formulation. The radioactive content must be within 90%-110% of the stated value at the date and time stated on the label.

### Example 1: Method of Manufacturing ¹⁷⁷LU-PSMA I&T Radiopharmaceutical Formulation

Multiple radiopharmaceutical compositions were produced using the process as outline in Table 2 below. Radiolabeling was performed using non-carrier added [¹⁷⁷Lu]LuCl₃. Compositions 1 and 2 are essentially the same. In Composition 3, the pH of the ascorbic acid solution was adjusted to 4.5, the amount of ascorbic acid was lowered, the amount of ethanol was lowered, and the pH of the final radiopharmaceutical composition was adjusted to 4.5. This resulted in Composition 3 having an extended shelf life as compared to Compositions 1 and 2.

The synthesis of ¹⁷⁷Lu-PSMA I&T takes place in an automated synthesis module in a controlled environment, used for the radiolabelling. The labelling solution containing [¹⁷⁷Lu]Lutetium chloride (¹⁷⁷LuCl₃) is connected to the synthesis cassette containing the other chemicals components required for the labelling process. ¹⁷⁷LuCl₃ solution is transferred into reactor for radiolabeling and may be rinsed with additional required amount of 0.04 M HCl solution. The labelling solutions varies in volume depending on the ¹⁷⁷Lu radioactivity.

The ¹⁷⁷LuCl₃ solution is mixed in the reaction chamber with a solution of 0.4 M Sodium acetate buffer containing the diluted PSMA I&T precursor. The solution is heated in the reactor. After heating the produced ¹⁷⁷Lu-PSMA I&T is trapped into a pre-conditioned (ethanol) C18 cartridge. The cartridge is rinsed with sterile water and after that the final product is eluted from the C18 cartridge with 1.5 ml of 50% sterile ethanol into a bulk vial. The drug substance is isolated in situ and directly formulated into the drug product.

For final volume adjustment a formulation matrix containing 50 mg/ml ascorbic acid and ethanol in injections grade water is added to the bulk vial. The composition of final product is fixed, and the amount of formulation matrix added depends on the radioactivity of ¹⁷⁷Lu used in the batch.

The formulation matrix is prepared from the injections grade solution of ascorbic acid that is diluted to 50 mg/ml concentration using injections grade water. The ethanol concentration is adjusted to 3.8% (v/v) to match the concentration of the synthesis bulk product regardless of dilution rate.

The synthesis is a one-step labelling process with C18 purification using injections grade ethanol and water as the only solvents, therefore no residual solvents are present.

Radiochemical impurities are quantified by chromatographical methods (HPLC and TLC). Radiochemical purity determined by HPLC must not be less than 95.0%

Depending on the total produced radioactivity the bulk product is diluted to a fixed radioactivity concentration of about 500 MBq/ml.

The solution is filtered through a 0.22 μm membrane filter into sterile product vials. In addition to patient doses, sample vials are also dispensed from each production batch (chemical QC sample, microbiological QC sample, and a reference sample for retention). The final product is dispensed in a Grade A controlled environment. The integrity of the filters is tested after filtration by performing a bubble point test prior to product release. The fill weight/volume and radioactivity is checked for dispensed patient vials. The solution is ready for use after pre-release quality control and QP release.

Radioactivity is monitored with a dose calibrator after the labelling process in order to ensure successful labelling and during dispensing to verify dispensed doses.

TABLE 2A
Radiolabeling Process

| | Process 1 | | Process 2 | | Process 3 | |
|---|---|---|---|---|---|---|
| Lu-177 | 40 GBq/ml in 0.04M HCl | | 40 GBq/ml in 0.04M HCl | | ~40 GBq/ml in 0.05M HCl | |
| PSMA-I&T precursor | 1 000 | μg/ml | 463 | μg/ml | 463 | μg/ml |
| Reaction Buffer | 4 ml 0.4M Sodium Acetate | | 4 ml 0.4M Sodium Acetate | | 4 ml 0.4M Sodium Acetate | |
| | 2 ml 0.04M HCl (0.08 | | 2 ml 0.04M HCl (0.08 | | 1.6 ml 0.05M HCl (0.08 | |
| | mmol HCl) | | mmol HCl) | | mmol HCl) | |
| | 150 μl 20% L-ascorbic | | 150 μl 20% L-ascorbic | | 150 μl 20% L-ascorbic | |
| | acid | | acid | | acid | |
| Reaction Volume | 6-8 | ml | 6-8 | ml | 6-8 | ml |

| | | | |
|---|---|---|---|
| Heating | Setpoint 110° C., 15 min | Setpoint 110° C., 15 min | Setpoint 110° C., 15 min |
| Purification | Sep Pak C18 | Sep Pak C18 | Sep Pak C18 |
| Elution | 1.5 ml Ethanol-Water 1:1 | 1.5 ml Ethanol-Water 1:1 | 1.5 ml Ethanol-Water 1:1 |
| Cassette flush | 8.5 ml Ascorbic acid 50 | 8.5 ml Ascorbic acid 50 | 8.5 ml Ascorbic acid 50 |
| | mg/ml (pH 5.5-7.0) | mg/ml (pH 5.5-7.0) | mg/ml, pH 4.5 |
| Formulation buffer | 42.5 mg/ml ascorbic acid, | 42.5 mg/ml ascorbic acid, | 31 mg/ml ascorbic acid, |
| | 7.5% Ethanol (v/v %), pH | 7.5% Ethanol (v/v %), pH | 3.8% Ethanol (v/v %), pH |
| | 5.5-7.0 | 5.5-7.0 | 4.5 |

The resulting Compositions 1-3 from Processes 1-3 are provided below in Table 2B, which provides the compositions for both a 1 ml volume and a vial of 10 ml or 20 ml for each composition.

TABLE 2B
¹⁷⁷Lu-PSMA I&T compositions 1-3.

| | Compositions 1 and 2 | | | | Composition 3 | | | | |
|---|---|---|---|---|---|---|---|---|---|
| | | | Quantity/10 mL | | | | Quantity/20 mL | | |
| Component | Quantity per mL | | vial | | Quantity per mL | | vial | | Function |
| ¹⁷⁷Lu-PSMA I&T | 1 GBq (27 | | 10 GBq (270 | | 0.5 GBq (13.5 | | 10 GBq (270 | | Radioactive |
| | mCi) | | mCi) | | mCi) | | mCi) | | pharmaceutical |
| | | | | | | | | | ingredient (exact |
| | | | | | | | | | activity/volume |
| | | | | | | | | | adjusted according |
| | | | | | | | | | to dose strength) |
| Lu-PSMA I&T | <12 | μg | <120 | μg | <6 | μg | <120 | μg | Mass of |
| | | | | | | | | | pharmaceutical |
| | | | | | | | | | ingredient |
| Ascorbic acid | 42.5 | mg | 425 | mg | 31 | mg | 620 | mg | Antioxidant |
| Disodium EDTA | 21 | μg | 210 | μg | 15.5 | μg | 310 | μg | Metal Ion chelator |

| Sodium | A sufficient | A sufficient | A sufficient | | A sufficient | | Present in |
|---|---|---|---|---|---|---|---|
| Bicarbonate | quantity | quantity | quantity | | quantity | | formulated Ascorbic |
| NaOH | A sufficient | A sufficient | A sufficient | | A sufficient | | acid Injection in |
| | quantity | quantity | quantity | | quantity | | sufficient amounts to |
| | | | | | | | control pH to 5.5-7.0 |
| Hydrochloric | None | None | 1.7 | mg/ml * | 34 | mg/ml * | *Theoretical amount. |

| | | | | | |
|---|---|---|---|---|---|
| acid | | | | | Added to adjust final |
| | | | | | formulation pH to 4.5 |
| Ethanol | 75 μL (58.9 mg) | 750 μL (589 mg) | 37.5 μL (29.5 mg) | 750 μL (589 mg) | Vehicle/stabilizing |
| | | | | | agent |
| Water for | Sufficient to make | Sufficient to make | Sufficient to make | Sufficient to make | Vehicle |
| Injection | 1 mL | 10 mL | 1 mL | 20 mL | |

### Example 2: Stability of the Radiochemical Compositions

Stability of ¹⁷⁷Lu-PSMA I&T composition 1 was tested and the radiochemical purity and chemical properties were shown to provide adequate stability of 48 hours from the end-of-synthesis time for samples stored at +5° C., +20° C. and +40° C., Table 3.

Stability studies show that ¹⁷⁷Lu-PSMA I&T composition 3 (Tables 4A-H) comprising ascorbic acid 31 mg/ml and ethanol 3.8% (v/v) at a pH of 4.5 had improved stability and extended shelf life compared to ¹⁷⁷Lu-PSMA I&T composition 1.

The radiochemical purity and chemical properties (pH, impurities, visual properties) of ¹⁷⁷Lu-PSMA I&T in the formulation composition 3 were tested on the seven batches over a time span of 70 to 72 hours from the end-of-synthesis time. Stability samples of typical therapeutic dose radioactivity and volume were stored in different conditions covering typical storage, shipment and usage of the product, including temperatures ranging from +5° C. to +40° C.

Final radioactivity concentration in the sample solutions varied from 497 MBq/ml to 642 MBq/ml at the end of dispensing.

All stability samples met the set acceptance criteria. Radiochemical purity was ≥95.7% after 70 hours or 72 hours after end of synthesis time in all samples analyzed.

Based on the results ¹⁷⁷Lu-PSMA I&T solution in formulation composition 3 was not found sensitive to the different storage conditions tested.

TABLE 3
Stability Data of ¹⁷⁷Lu-PSMA I&T composition 1.

| Stability criteria | | Stability data at 48 h after end of synthesis | | | | |
|---|---|---|---|---|---|---|
| Test | Specification | Batch #1 | Batch #2 | Batch #3, +5° C. | Batch #3, +20° C. | Batch #3, +40° C. |
| Visual | Clear, | Pass | Pass | Pass | Pass | Pass |
| inspection | colorless | | | | | |
| | or slightly | | | | | |
| | yellow liquid, | | | | | |
| | no visible | | | | | |
| | particles | | | | | |
| pH | 5.0-8.0 | 6.0 | 5.5 | 6.0 | 6.0 | 5.5 |
| Radiochemical | ≥95.0% as | 97.4% | 96.2% | 95.6% | 96.1 | 95.1% |
| Purity (HPLC), | ¹⁷⁷Lu-PSMA | | | | | |
| % | I&T | | | | | |
| Radiochemical | ¹⁷⁷Lu-colloid ≤5.0% | 0% | 0% | 0% | 0% | 0% |
| Purity by TLC, % | of radioactivity | | | | | |
| Chemical purity | Lu-PSMA I&T - | 1.6 μg/ml | 2.2 μg/ml | 1.9 μg/ml | 1.8 μg/ml | 1.8 μg/ml |
| Lu-PSMA I&T | concentration ≤12.0 | | | | | |
| | μg/ml | | | | | |

TABLE 4A
Chemical Quality in the Validation Batches of ¹⁷⁷Lu-PSMA I&T composition 3.

| QC TESTING | | | | |
|---|---|---|---|---|
| Validation criteria | | VALIDATION BATCH # | | |
| Test | Specification | 1 | 2 | 3 |
| Visual inspection | Clear, colorless | Pass | Pass | Pass |
| | to yellow | | | |
| | solution, no | | | |
| | visible particles | | | |
| Radiochemical | Co-elutes with | Pass | Pass | Pass |
| Identity | reference | | | |
| | standard ^{†} | | | |
| pH | 4.0-5.0 | 4.5 | 4.5 | 4.5 |
| Radionuclidic | Gamma ray | 113 keV and | 113 keV and | 113 keV and |
| Identification keV | energy | 209 keV | 209 keV | 209 keV |
| | 113 ± 2 keV | no other | no other | no other |
| | 208 ± 4 keV | significant | significant | significant |
| | (HPGe-detector) | peaks | peaks | peaks |
| | No other | detected | detected | detected |
| | significant peaks | | | |
| | with gamma | | | |
| | energy >100 keV | | | |
| | are detected | | | |
| Radiochemical Purity | ≥95.0% as | 98.2% | 98.2% | 97.9% |
| (HPLC), % | ¹⁷⁷Lu-PSMA I&T | | | |
| Radiochemical Purity | ¹⁷⁷Lu-colloid ≤5.0% | 0% | 0% | 0% |
| by TLC, % | of radioactivity | | | |
| Ethanol content, % | 2.5-4.5% | 3.5% | 3.5% | 3.2% |
| (v/v) (GC) | | | | |
| Chemical purity | Lu-PSMA I&T - | 1.2 | 1.2 | 1.2 |
| Lu-PSMA I&T | concentration ≤6.0 | μg/ml | μg/ml | μg/ml |
| | μg/ml | | | |
| ¹⁷⁷Lu-PSMA-I&T | Reported | 271 | 275 | 191 |
| Specific activity | | GBq/μmol | GBq/μmol | GBq/μmol |
| Ascorbic acid | 15-35 | 25 | 25 | 25 |
| concentration | mg/ml | mg/ml | mg/ml | mg/ml |
| mg/ml | | | | |
| Radionuclidic purity | ≥99.9% of the | 1000% | 100.0% | 100.0% |
| | total activity of | | | |
| | ¹⁷⁷Lu | | | |
| Bacterial endotoxins | <175 EU/20 ml | <5.00 | <5.00 | <5.00 |
| EU/ml | (measured <8.75 | EU/ml | EU/ml | EU/ml |
| | (EU/ml)) | | | |
| Filter integrity test | ≥3.32 bar tested | Pass | Pass | Pass |
| | with water | | | |
| Sterility | Sterile | Pass | Pass | Pass |

^{†} The radiometric RT of the test sample = UV RT of Reference Standard ± 5%.

TABLE 4B
Stability Data of ¹⁷⁷Lu-PSMA I&T composition 3.

| STABILITY BATCH #1 | 70 h post End of Synthesis | | | | | | | |
|---|---|---|---|---|---|---|---|---|
| VIAL # | 1 | | 2 | | 3 | | 4 | |
| Radioactivity | 9430 | MBq | 9440 | MBq | 9440 | MBq | 9430 | MBq |
| Fill Volume | 18.04 | ml | 18.10 | ml | 18.07 | ml | 18.03 | ml |

| | | | | | | | |
|---|---|---|---|---|---|---|---|
| Storage Condition | +22.5° | C. | +32.5° | C. | +22.5° C./ | +22.5° | C. |

Inverted

| QC TESTING | | | | | |
|---|---|---|---|---|---|
| Validation | | | | | |
| criteria | | | | | |
| Test | Specification | ZZZZZ | ZZZZZ | ZZZZZ | ZZZZZ |
| Visual | Clear, colorless | Pass | Pass | Pass | Pass |
| inspection | to yellow | | | | |
| | solution, no | | | | |
| | visible particles | | | | |
| Radiochemical | Co-elutes with | Pass | Pass | Pass | Pass |
| Identity | reference | | | | |
| | standard ^{†} | | | | |
| pH | 4.0-5.0 | 4.5 | 4.5 | 4.5 | 4.5 |
| Radionuclidic | Gamma ray | 113 keV and | 113 keV and | 113 keV and | 113 keV and |
| Identification | energy | 209 keV | 209 keV | 209 keV | 209 keV |
| keV | 113 ± 2 keV | no other | no other | no other | no other |
| | 208 ± 4 keV | significant | significant | significant | significant |
| | (HPGe- | peaks | peaks | peaks | peaks |
| | detector) | detected | detected | detected | detected |
| | No other | | | | |
| | significant | | | | |
| | peaks with | | | | |
| | gamma energy >100 | | | | |
| | keV are detected | | | | |
| Radiochemical | ≥95.0% as | 96.7% | 96.2% | 95.9% | 96.0% |
| Purity (HPLC), | ¹⁷⁷Lu-PSMA | | | | |
| % | I&T | | | | |
| Radiochemical | ¹⁷⁷Lu-colloid ≤5.0% | 0% | 0% | 0% | 0% |
| Purity by TLC, | of radioactivity | | | | |
| % | | | | | |
| Ethanol | 2.5-4.5% | N/A | N/A | N/A | N/A |
| content, % | | | | | |
| (v/v) (GC) | | | | | |
| Chemical | Lu-PSMA I&T - | 1.3 | 1.3 | 1.2 | 1.3 |
| purity | concentration ≤6.0 | μg/ml | μg/ml | μg/ml | μg/ml |
| Lu-PSMA I&T | μg/ml | | | | |
| Ascorbic acid | 15-35 | 25 | 25 | 25 | 25 |
| concentration | mg/ml | mg/ml | mg/ml | mg/ml | mg/ml |
| mg/ml | | ZZZZZ | ZZZZZ | ZZZZZ | ZZZZZ |

^{†} The radiometric RT of the test sample = UV RT of Reference Standard ± 5%.

TABLE 4C
Stability Data of ¹⁷⁷Lu-PSMA I&T composition 3.

| STABILITY BATCH #2 | 72 h post End of Synthesis | | | | | | | |
|---|---|---|---|---|---|---|---|---|
| VIAL # | 1 | | 2 | | 3 | | 4 | |
| Radioactivity | 9650 | MBq | 9550 | MBq | 9540 | MBq | 9540 | MBq |
| Fill Volume | 15.02 | ml | 14.86 | ml | 14.86 | ml | 14.87 | ml |

| | | | | | | | |
|---|---|---|---|---|---|---|---|
| Storage Condition | +22.5° | C. | +32.5° | C. | +22.5° C./ | +22.5° | C. |

Inverted

| QC TESTING | | | | | |
|---|---|---|---|---|---|
| Validation | | | | | |
| criteria | | | | | |
| Test | Specification | ZZZZZ | ZZZZZ | ZZZZZ | ZZZZZ |
| Visual | Clear, colorless | Pass | Pass | Pass | Pass |
| inspection | to yellow | | | | |
| | solution, no | | | | |
| | visible particles | | | | |
| Radiochemical | Co-elutes with | Pass | Pass | Pass | Pass |
| Identity | reference | | | | |
| | standard ^{†} | | | | |
| pH | 4.0-5.0 | 4.5 | 4.5 | 4.5 | 4.5 |
| Radionuclidic | Gamma ray | 113 keV and | 113 keV and | 113 keV and | 113 keV and |
| Identification | energy | 209 keV | 209 keV | 209 keV | 209 keV |
| keV | 113 ± 2 keV | no other | no other | no other | no other |
| | 208 ± 4 keV | significant | significant | significant | significant |
| | (HPGe- | peaks | peaks | peaks | peaks |
| | detector) | detected | detected | detected | detected |
| | No other | | | | |
| | significant | | | | |
| | peaks with | | | | |
| | gamma energy >100 | | | | |
| | keV are detected | | | | |
| Radiochemical | ≥95.0% as | 96.0% | 95.7% | 961.% | 96.1% |
| Purity (HPLC), | ¹⁷⁷Lu-PSMA | | | | |
| % | I&T | | | | |
| Radiochemical | ¹⁷⁷Lu-colloid ≤5.0% | 0% | 0% | 0% | 0% |
| Purity by TLC, | of | | | | |
| % | radioactivity | | | | |
| Ethanol | 2.5-4.5% | N/A | N/A | N/A | N/A |
| content, % | | | | | |
| (v/v) (GC) | | | | | |
| Chemical | Lu-PSMA I&T - | 1.2 | 1.2 | 1.2 | 1.1 |
| purity | concentration ≤6.0 | μg/ml | μg/ml | μg/ml | μg/ml |
| Lu-PSMA I&T | μg/ml | | | | |
| Ascorbic acid | 15-35 | 25 | 25 | 25 | 25 |
| concentration | mg/ml | mg/ml | mg/ml | mg/ml | mg/ml |
| mg/ml | | | | | |

^{†} The radiometric RT of the test sample = UV RT of Reference Standard ± 5%.

TABLE 4D
Stability Data of ¹⁷⁷Lu-PSMA I&T composition 3.

| STABILITY BATCH #3 | 72 h post End of Synthesis | | | | | | | |
|---|---|---|---|---|---|---|---|---|
| VIAL # | 1 | | 2 | | 3 | | 4 | |
| Radioactivity | 9840 | MBq | 9540 | MBq | 9530 | MBq | 9530 | MBq |
| Fill Volume | 17.51 | ml | 17.01 | ml | 16.99 | ml | 16.98 | ml |

| | | | | | | | |
|---|---|---|---|---|---|---|---|
| Storage Condition | +22.5° | C. | +32.5° | C. | ≥75% RH | +5° | C. |

(Ambient)

| QC TESTING | | | | | |
|---|---|---|---|---|---|
| Validation | | | | | |
| criteria | | | | | |
| Test | Specification | ZZZZZ | ZZZZZ | ZZZZZ | ZZZZZ |
| Visual | Clear, colorless | Pass | Pass | Pass | Pass |
| inspection | to yellow | | | | |
| | solution, no | | | | |
| | visible particles | | | | |
| Radiochemical | Co-elutes with | Pass | Pass | Pass | Pass |
| Identity | reference | | | | |
| | standard ^{†} | | | | |
| pH | 4.0-5.0 | 4.5 | 4.5 | 4.5 | 4.5 |
| Radionuclidic | Gamma ray | 113 keV and | 113 keV and | 113 keV and | 113 keV and |
| Identification | energy | 209 keV | 209 keV | 209 keV | 209 keV |
| keV | 113 ± 2 keV | no other | no other | no other | no other |
| | 208 ± 4 keV | significant | significant | significant | significant |
| | (HPGe- | peaks | peaks | peaks | peaks |
| | detector) | detected | detected | detected | detected |
| | No other | | | | |
| | significant | | | | |
| | peaks with | | | | |
| | gamma energy >100 | | | | |
| | keV are detected | | | | |
| Radiochemical | ≥95.0% as | 95.8% | 96.0% | 96.1% | 96.0% |
| Purity (HPLC), | ¹⁷⁷Lu-PSMA | | | | |
| % | I&T | | | | |
| Radiochemical | ¹⁷⁷Lu-colloid ≤5.0% | 0% | 0% | 0% | 0% |
| Purity by TLC, | of | | | | |
| % | radioactivity | | | | |
| Ethanol | 2.5-4.5% | N/A | N/A | N/A | N/A |
| content, % | | | | | |
| (v/v) (GC) | | | | | |
| Chemical | Lu-PSMA I&T - | 1.0 | 0.9 | 1.0 | 1.0 |
| purity | concentration ≤6.0 | μg/ml | μg/ml | μg/ml | μg/ml |
| Lu-PSMA I&T | μg/ml | | | | |
| Ascorbic acid | 15-35 | 15 | 15 | 15 | 15 |
| concentration | mg/ml | mg/ml | mg/ml | mg/ml | mg/ml |
| mg/ml | | | | | |

^{†} The radiometric RT of the test sample = UV RT of Reference Standard ± 5%.

TABLE 4E
Stability Data of ¹⁷⁷Lu-PSMA I&T composition 3.
STABILITY BATCH #4 72 h post End of Synthesis

| | | VIAL # | | | | | | | | | |
|---|---|---|---|---|---|---|---|---|---|---|---|
| | | 1 | | 2 | | 3 | | 4 | | 5 | |
| Radioactivity | | 9490 | MBq | 9600 | MBq | 9600 | MBq | 9600 | MBq | 9590 | MBq |
| Fill Volume | | 17.19 | ml | 17.42 | ml | 17.45 | ml | 17.44 | ml | 17.42 | ml |
| Storage Condition | | +22.5° | C. | +32.5° | C. | +40° | C. | +5° | C. | +5° | C. |
| QC TESTING | | | | | | | | | | | |
| Validation | | | | | | | | | | | |
| criteria | | | | | | | | | | | |
| Test | Specification | | | | | | | | | | |
| Visual | Clear, | Pass | | Pass | | Pass | | Pass | | Pass | |
| inspection | colorless to | | | | | | | | | | |
| | yellow | | | | | | | | | | |
| | solution, no | | | | | | | | | | |
| | visible | | | | | | | | | | |
| | particles | | | | | | | | | | |
| Radiochemical | Co-elutes | Pass | | Pass | | Pass | | Pass | | Pass | |
| Identity | with | | | | | | | | | | |
| | reference | | | | | | | | | | |
| | standard ^{†} | | | | | | | | | | |
| pH | 4.0-5.0 | 4.5 | | 4.5 | | 4.5 | | 4.5 | | 4.5 | |
| Radionuclidic | Gamma ray | 113 keV | | 113 keV | | 113 keV | | 113 keV | | 113 keV | |
| Identification | energy | and | | and | | and | | and | | and | |
| keV | 113 ± 2 keV | 209 keV | | 209 keV | | 209 keV | | 209 keV | | 209 keV | |
| | 208 ± 4 keV | no other | | no other | | no other | | no other | | no other | |
| | (HPGe- | significant | | significant | | significant | | significant | | significant | |
| | detector) | peaks | | peaks | | peaks | | peaks | | peaks | |
| | No other | detected | | detected | | detected | | detected | | detected | |
| | significant | | | | | | | | | | |
| | peaks with | | | | | | | | | | |
| | gamma | | | | | | | | | | |
| | energy >100 | | | | | | | | | | |
| | keV are | | | | | | | | | | |
| | detected | | | | | | | | | | |
| Radiochemical | ≥95.0% as | 96.5% | | 95.9% | | 95.8% | | 96.3% | | 96.6% | |
| Purity (HPLC), % | ¹⁷⁷Lu-PSMA I&T | | | | | | | | | | |
| Radiochemical | ¹⁷⁷Lu-colloid ≤5.0% | 0% | | 0% | | 0% | | 0% | | 0% | |
| Purity by TLC, % | of radioactivity | | | | | | | | | | |
| Ethanol | 2.5-4.5% | N/A | | N/A | | N/A | | N/A | | N/A | |
| content, % | | | | | | | | | | | |
| (v/v) (GC) | | | | | | | | | | | |
| Chemical | Lu-PSMA I&T - | 1.0 | μg/ml | 1.0 | μg/ml | 1.0 | μg/ml | 1.0 | μg/ml | 1.0 | μg/ml |
| purity | concentration ≤6.0 | | | | | | | | | | |
| Lu-PSMA I&T | μg/ml | | | | | | | | | | |
| Ascorbic acid | 15-35 mg/ml | 25 | mg/ml | 25 | mg/ml | 25 | mg/ml | 25 | mg/ml | 25 | mg/ml |

concentration
mg/ml

^{†} The radiometric RT of the test sample = UV RT of Reference Standard ± 5%.

TABLE 4F
Stability Data of ¹⁷⁷Lu-PSMA I&T composition 3.

| STABILITY BATCH #5 | 72 h post End of Synthesis | | | | | | | |
|---|---|---|---|---|---|---|---|---|
| VIAL # | 1 | | 2 | | 3 | | 4 | |
| Radioactivity | 9930 | MBq | 9800 | MBq | 9800 | MBq | 9810 | MBq |
| Fill Volume | 19.91 | ml | 19.69 | ml | 19.68 | ml | 19.73 | ml |
| Storage Condition | +22.5° | C. | +32.5° | C. | +5° | C. | +5° | C. |

| QC TESTING | | | | | |
|---|---|---|---|---|---|
| Validation | | | | | |
| criteria | | | | | |
| Test | Specification | ZZZZZ | ZZZZZ | ZZZZZ | ZZZZZ |
| Visual | Clear, colorless | Pass | Pass | Pass | Pass |
| inspection | to yellow | | | | |
| | solution, no | | | | |
| | visible particles | | | | |
| Radiochemical | Co-elutes with | Pass | Pass | Pass | Pass |
| Identity | reference | | | | |
| | standard ^{†} | | | | |
| pH | 4.0-5.0 | 4.5 | 4.5 | 4.5 | 4.5 |
| Radionuclidic | Gamma ray | 113 keV and | 113 keV and | 113 keV and | 113 keV and |
| Identification | energy | 209 keV | 209 keV | 209 keV | 209 keV |
| keV | 113 ± 2 keV | no other | no other | no other | no other |
| | 208 ± 4 keV | significant | significant | significant | significant |
| | (HPGe-detector) | peaks | peaks | peaks | peaks |
| | No other | detected | detected | detected | detected |
| | significant peaks | | | | |
| | with gamma | | | | |
| | energy >100 | | | | |
| | keV are detected | | | | |
| Radiochemical | ≥95.0% as | 97.0% | 97.0% | 96.8% | 97.3% |
| Purity (HPLC), | ¹⁷⁷Lu-PSMA I&T | | | | |
| % | | | | | |
| Radiochemical | ¹⁷⁷Lu-colloid ≤5.0% | 0% | 0% | 0% | 0% |
| Purity by TLC, | of | | | | |
| % | radioactivity | | | | |
| Ethanol | 2.5-4.5% | N/A | N/A | N/A | N/A |
| content, % (v/v) | | | | | |
| (GC) | | | | | |
| Chemical | Lu-PSMA I&T - | 0.9 | 0.9 | 0.9 | 0.9 |
| purity | concentration ≤6.0 | μg/ml | μg/ml | μg/ml | μg/ml |
| Lu-PSMA I&T | μg/ml | | | | |
| Ascorbic acid | 15-35 | 25 | 25 | 25 | 25 |
| concentration | mg/ml | mg/ml | mg/ml | mg/ml | mg/ml |
| mg/ml | | | | | |

^{†} The radiometric RT of the test sample = UV RT of Reference Standard ± 5%.

TABLE 4G
Stability Data of ¹⁷⁷Lu-PSMA I&T composition 3.

| STABILITY BATCH #6 | 72 h post End of Synthesis | | |
|---|---|---|---|
| VIAL # | | 1 | 4 * |

| | | | | |
|---|---|---|---|---|
| Radioactivity | 9960 | MBq | 9800 | MBq |
| Fill Volume | 19.69 | ml | 19.40 | ml |
| Storage Condition | +22.5° | C. | +40° | C. |

| QC TESTING | | | |
|---|---|---|---|
| Validation criteria | | | |
| Test | Specification | ZZZZZ | ZZZZZ |
| Visual inspection | Clear, colorless to yellow | Pass | Pass |
| | solution, no visible | | |
| | particles | | |
| Radiochemical Identity | Co-elutes with | Pass | Pass |
| | reference standard ^{†} | | |
| pH | 4.0-5.0 | 4.5 | 4.5 |
| Radionuclidic | Gamma ray energy | 113 keV and | 113 keV and |
| Identification | 113 ± 2 keV | 209 keV | 209 keV |
| keV | 208 ± 4 keV | no other significant | no other significant |
| | (HPGe-detector) | peaks detected | peaks detected |
| | No other significant | | |
| | peaks with gamma | | |
| | energy >100 keV are | | |
| | detected | | |
| Radiochemical Purity | ≥95.0% as ¹⁷⁷Lu-PSMA | 96.7% | 95.8% |
| (HPLC), % | I&T | | |
| Radiochemical Purity | ¹⁷⁷Lu-colloid ≤5.0% of | 0% | 0% |
| by TLC, % | radioactivity | | |
| Ethanol content, % | 2.5-4.5% | N/A | N/A |
| (v/v) (GC) | | | |
| Chemical purity | Lu-PSMA I&T - | 1.0 | 1.0 |
| Lu-PSMA I&T | concentration ≤6.0 μg/ml | μg/ml | μg/ml |
| Ascorbic acid | 15-35 | 25 | 25 |
| concentration | mg/ml | mg/ml | mg/ml |
| mg/ml | | | |

^{†} The radiometric RT of the test sample = UV RT of Reference Standard ± 5%.

TABLE 4H
Stability Data of ¹⁷⁷Lu-PSMA I&T composition 3.

| STABILITY BATCH #7 | 72 h post End of Synthesis | | | |
|---|---|---|---|---|
| VIAL # | | 1 | 2 | 3 |

| | | | | | | |
|---|---|---|---|---|---|---|
| Radioactivity | 9870 | MBq | 9880 | MBq | 9910 | MBq |
| Fill Volume | 19.31 | ml | 19.34 | ml | 19.43 | ml |

| | | | |
|---|---|---|---|
| Storage Condition | +22.5° C./ | +32.5° C./ | +5° C./ |
| | Inverted | Inverted | Inverted |

| QC TESTING | | | | |
|---|---|---|---|---|
| Validation criteria | | | | |
| Test | Specification | ZZZZZ | ZZZZZ | ZZZZZ |
| Visual inspection | Clear, colorless to | Pass | Pass | Pass |
| | yellow solution, no | | | |
| | visible particles | | | |
| Radiochemical | Co-elutes with | Pass | Pass | Pass |
| Identity | reference standard ^{†} | | | |
| pH | 4.0-5.0 | 4.5 | 4.5 | 4.5 |
| Radionuclidic | Gamma ray energy | 113 keV and | 113 keV and | 113 keV and |
| Identification | 113 ± 2 keV | 209 keV | 209 keV | 209 keV |
| keV | 208 ± 4 keV | no other | no other | no other |
| | (HPGe-detector) | significant peaks | significant peaks | significant peaks |
| | No other significant | detected | detected | detected |
| | peaks with gamma | | | |
| | energy >100 keV | | | |
| | are detected | | | |
| Radiochemical | ≥95.0% as ¹⁷⁷Lu- | 96.5% | 96.4% | 96.3% |
| Purity (HPLC), % | PSMA I&T | | | |
| Radiochemical | ¹⁷⁷Lu-colloid ≤5.0% | 0% | 0% | 0% |
| Purity by TLC, % | of radioactivity | | | |
| Ethanol content, | 2.5-4.5% | N/A | N/A | N/A |
| % (v/v) (GC) | | | | |
| Chemical purity | Lu-PSMA I&T - | 1.0 | 0.9 | 1.0 |
| Lu-PSMA I&T | concentration ≤6.0 | μg/ml | μg/ml | μg/ml |
| | μg/ml | | | |
| Ascorbic acid | 15-35 | 25 | 25 | 25 |
| concentration | mg/ml | mg/ml | mg/ml | mg/ml |
| mg/ml | | | | |

^{†} The radiometric RT of the test sample = UV RT of Reference Standard ± 5%.

Specifications for the ¹⁷⁷Lu-PSMA A&T solution are presented in the Table 5 below. The specifications listed were used as release parameters except for sterility testing. Sterility was tested on all batches post-release.

TABLE 5
Specifications for ¹⁷⁷Lu-PSMA I&T

| | Specification | Specification |
|---|---|---|
| Characteristics | (Composition 1) | (Composition 3) |
| Appearance | Clear, colorless or | Clear, colorless or |
| | slightly yellow liquid, | slightly yellow liquid, |
| | no visible particles | no visible particles |
| pH | 5.0-8.0 | 4.0-5.0 |
| Ascorbic Acid | 20-70 mg/ml | 15-35 mg/ml |
| Ethanol | 5.0% to 8.0% | 2.5% to 4.5% |
| Radionuclidic | Gamma ray energy | Gamma ray energy |
| identification | 113 ± 10 KeV and | 113 ± 10 KeV and |
| | 208 ± 12 KeV | 208 ± 12 KeV |
| Radiochemical Identity | The radiometric RT | The radiometric RT |
| | of the test sample = | of the test sample = |
| | UV RT of Reference | UV RT of Reference |
| | Standard ±5%. | Standard ±5%. |
| Radiochemical purity | ≥95.0% | ≥95.0% |
| by HPLC method | | |
| Radiochemical purity | ¹⁷⁷Lu-colloid ≤5.0% | ¹⁷⁷Lu-colloid ≤5.0% |
| by TLC method | | |
| Chemical purity | Lu-PSMA I&T - | Lu-PSMA I&T - |
| | concentration ≤12.0 | concentration ≤12.0 |
| | μg/mL | μg/mL |
| Radionuclidic purity | ≥99.9% of the total | ≥99.9% of the total |
| | activity of ¹⁷⁷ Lu at | activity of ¹⁷⁷Lu at |
| | the end of the | the end of the |
| | operating life | operating life |
| ¹⁷⁷Lu-PSMA-I&T | Reported | Reported |
| Specific activity | | |
| Endotoxins | <17.5 IU/ml | <17.5 IU/ml |
| Radioactive content | 90%-110% at the | 90%-110% at the |
| | date and time | date and time stated |
| | stated on the label | on the label |
| Sterility | Sterile | Sterile |

### Example 3: Radiochemical Purity of ¹⁷⁷Lu-PSMA I&T in Different Formulations Compositions

The example demonstrates radiochemical stability of ¹⁷⁷Lu-PSMA I&T in formulation compositions at different pH values. The shelf life of ¹⁷⁷Lu-PSMA I&T is restricted by high rate of radiolysis during preparation and storage resulting in decomposition of ¹⁷⁷Lu-PSMA I&T and formation of radiochemical impurities. This eventually results in radiochemical purity of ¹⁷⁷Lu-PSMA I&T solution to fall below acceptance limit of 95.0%.

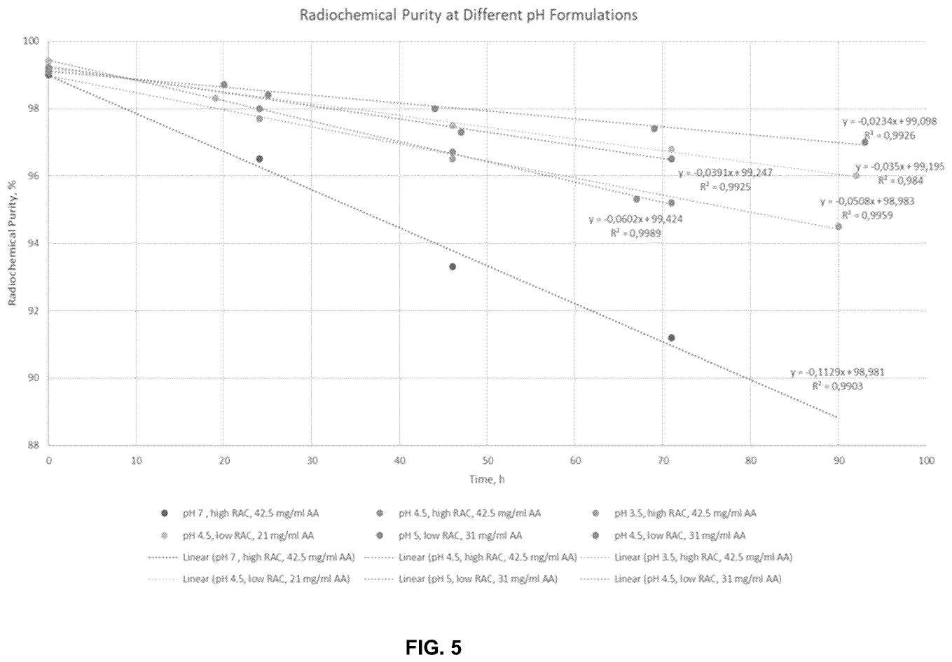

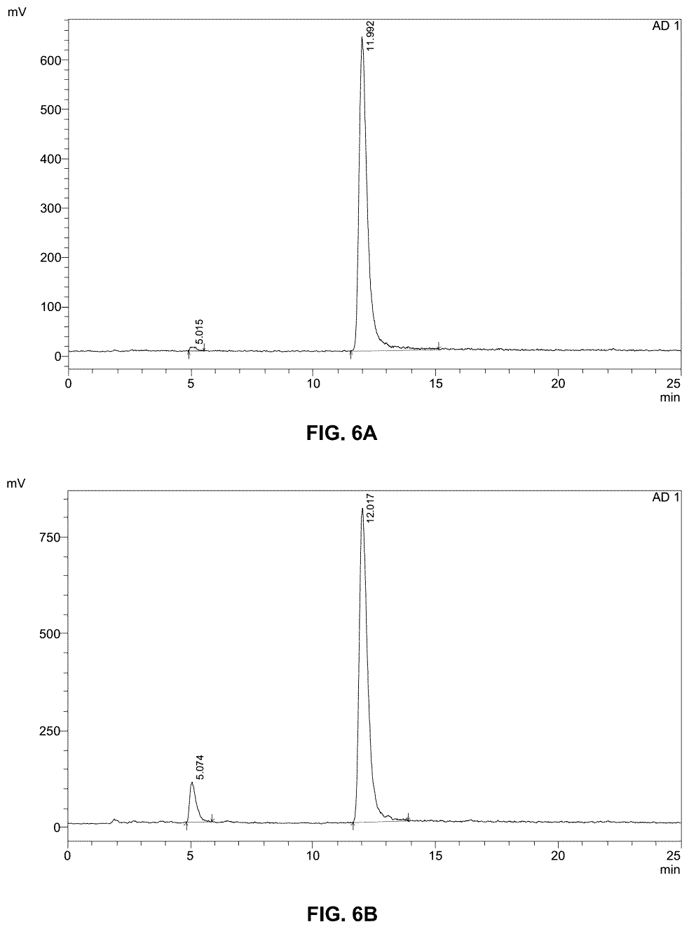

Formation of a particular radiochemical impurity of ¹⁷⁷Lu-PSMA I&T have been observed having retention time of about 5.2 minutes by HPLC with Phenomenex Luna C18 column (3 μm, 150 mm×4.6 mm) using 0.1% trifluoroacetic acid in water (Mobile phase A) and 0.1% trifluoroacetic acid in water:acetonitrile (10:90% v/v) (Mobile phase B) and isocratic method of 23% Mobile phase B at temperature of 40° C. The impurity referred herein having retention time of about 5.2 minutes is exemplified in the chromatograms in FIGS. 6A-11B.

In experiments conducted previously, lowering formulation radioactivity concentration was not found adequate reducing formation of the impurity eluting at about 5.2 minutes and maintaining radiochemical stability of ¹⁷⁷Lu-PSMA I&T solutions above 95.0% for 72 hours.

In this example, six experiments were conducted where ¹⁷⁷Lu-PSMA I&T was prepared in different formulation compositions with varying ascorbic acid concentration, pH and radioactivity concentration. Product formulation details are described in Table 6.

TABLE 6
¹⁷⁷Lu-PSMA I&T formulation compositions evaluated

| Experiment | Formulation | | Sample | Sample | Radioactivity | Storage |
|---|---|---|---|---|---|---|
| no. | composition | pH | Radioactivity | Volume | Concentration | Temperature |
| 1 | high RAC, 42.5 | 7 | 12 810 MBq | 10 ml | 1281 MBq/ml | 22.5° C. |
| | mg/ml Ascorbic acid | | | | | |
| 2 | high RAC, 42.5 | 4.5 | 12 780 MBq | 10 ml | 1278 MBq/ml | 22.5° C. |
| | mg/ml Ascorbic acid | | | | | |
| 3 | high RAC, 42.5 | 3.5 | 13 110 MBq | 10 ml | 1311 MBq/ml | 22.5° C. |
| | mg/ml Ascorbic acid | | | | | |
| 4 | low RAC, 31 mg/ml | 4.5 | 11 580 MBq | 20 ml | 579 MBq/ml | 22.5° C. |
| | Ascorbic acid | | | | | |
| 5 | low RAC, 31 mg/ml | 5 | 12 520 MBq | 20 ml | 626 MBq/ml | 22.5° C. |
| | Ascorbic acid | | | | | |
| 6 | low RAC, 31 mg/ml | 4.5 | 11 770 MBq | 20 ml | 589 MBq/ml | 22.5° C. |
| | Ascorbic acid | | | | | |

High radioactivity concentration (high RAC) in sample solutions were 1278 MBq/ml, 1281 MBq/ml and 1311 MBq/ml measured at the end of production. Low radioactivity concentration (low RAC) in sample solutions were 579 MBq/ml, 589 MBq/ml and 626 MBq/ml measured at the end of production. Radiochemical purity of each solution was followed by HPLC up to 71-93 hours post-radiolabeling. All solutions were stored at 22.5° C.

FIG. 5 shows results of radiochemical purity analyses at different time points determined by HPLC.

FIG. 6A and FIG. 6B show HPLC radio-chromatograms of experiment 1 at 0 and 71 hours post EOS, respectively.

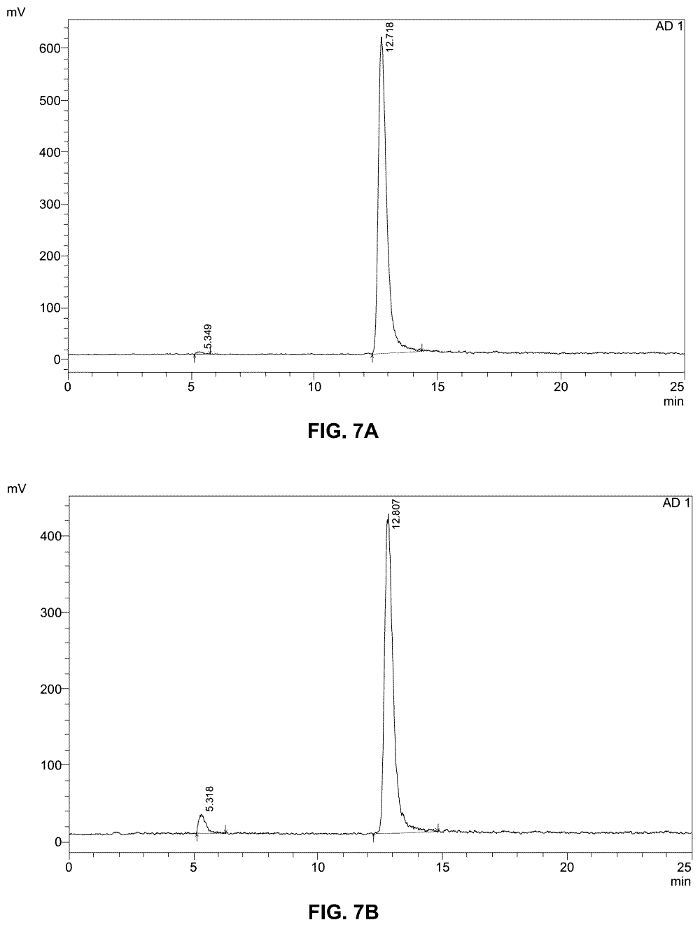

FIG. 7A and FIG. 7B show HPLC radio-chromatograms of experiment 2 at 0 and 71 hours post EOS, respectively.

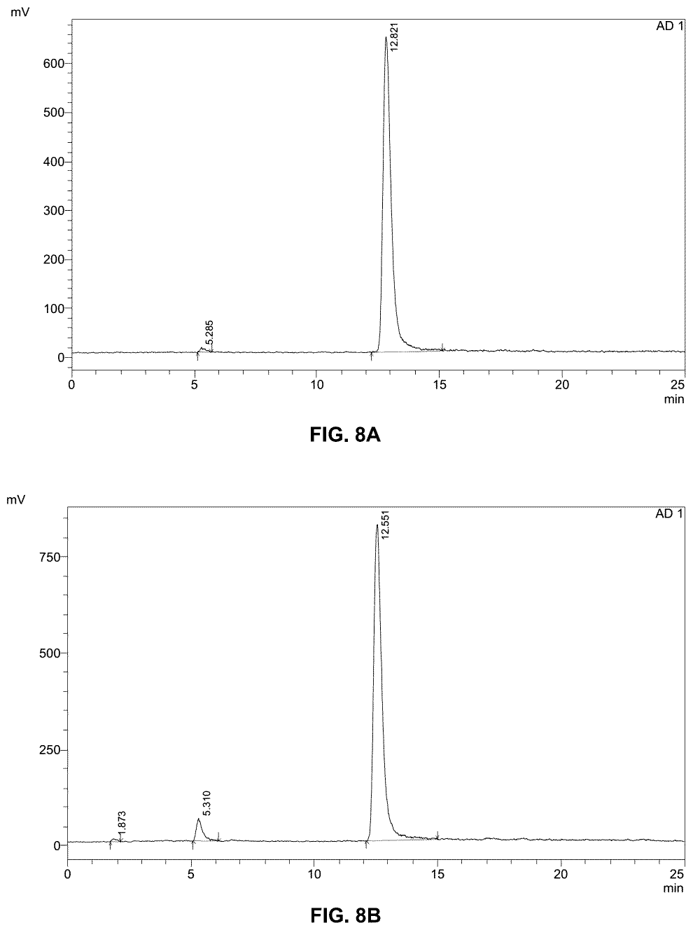

FIG. 8A and FIG. 8B show HPLC radio-chromatograms of experiment 3 at 0 and 90 hours post EOS, respectively.

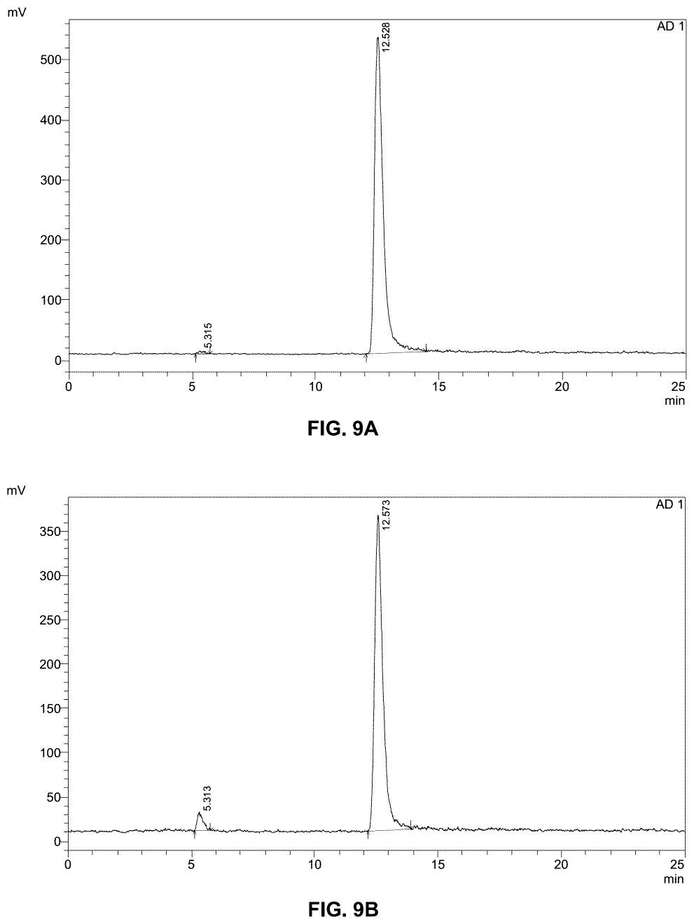

FIG. 9A and FIG. 9B show HPLC radio-chromatograms of experiment 4 at 0 and 92 hours post EOS, respectively.

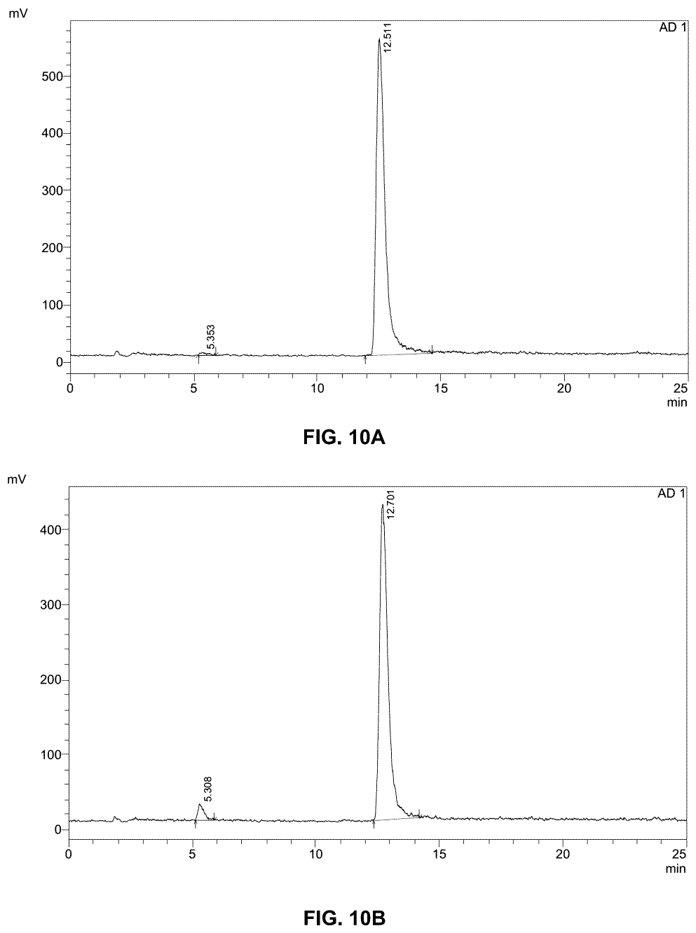

FIG. 10A and FIG. 10B show HPLC radio-chromatograms of experiment 5 at 0 and 71 hours post EOS, respectively.

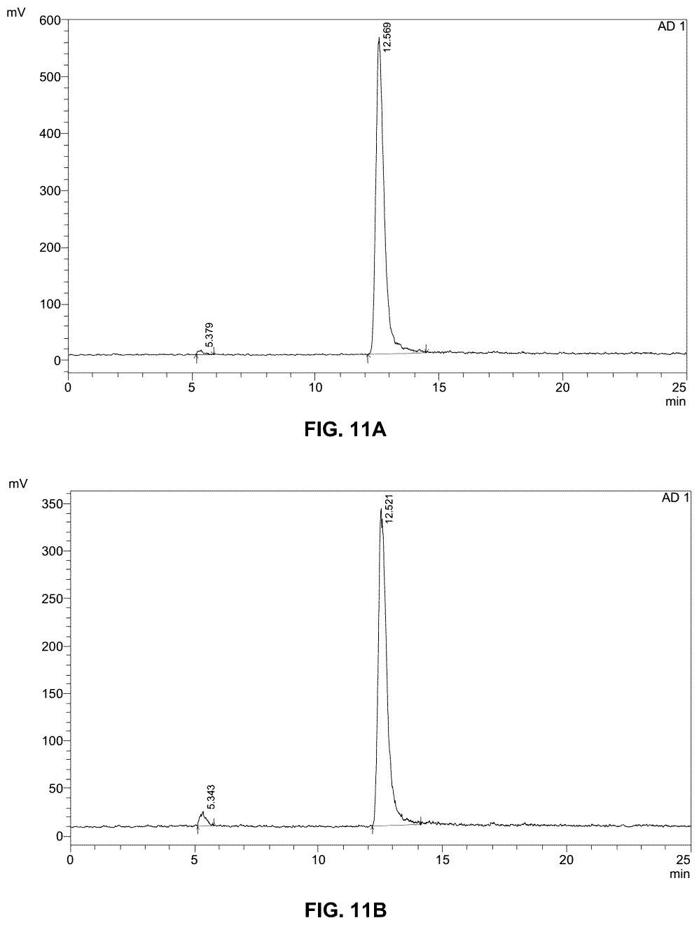

FIG. 11A and FIG. 11B show HPLC radio-chromatograms of experiment 6 at 0 and 93 hours post EOS, respectively.

The radiochemical stability results for each experiment at different time points are provided in Tables 7-12.

TABLE 7
Experiment 1
Radiochemical purity by HPLC (hours post EOS)

| T = 0 h | T = 24 h | T = 46 h | T = 64 h | T = 71 h |
|---|---|---|---|---|
| 99.0% | 96.5% | 93.3% | N/A | 91.2% |

TABLE 8
Experiment 2
Radiochemical purity by HPLC (hours post EOS)

| T = 0 h | T = 24 h | T = 46 h | T = 67 h | T = 71 h |
|---|---|---|---|---|
| 99.4% | 98.0% | 96.7% | 95.3% | 95.2% |

TABLE 9
Experiment 3
Radiochemical purity by HPLC (hours post EOS)

| T = 0 h | T = 24 h | T = 46 h | T = 64 h | T = 72 h | T = 90 h |
|---|---|---|---|---|---|
| 99.1% | 97.7% | 96.5% | N/A | N/A | 94.5% |

TABLE 10
Experiment 4
Radiochemical purity by HPLC (hours post EOS)

| T = 0 h | T = 19 h | T = 46 h | T = 71 h | T = 92 h |
|---|---|---|---|---|
| 99.4% | 98.3% | 97.5% | 96.8% | 96.0% |

TABLE 11
Experiment 5
Radiochemical purity by HPLC (hours post EOS)

| T = 0 h | T = 25 h | T = 47 h | T = 71 h |
|---|---|---|---|
| 99.2% | 98.4% | 97.3% | 96.5% |

TABLE 12
Experiment 6
Radiochemical purity by HPLC (hours post EOS)

| T = 0 h | T = 20 h | T = 44 h | T = 69 h | T = 93 h |
|---|---|---|---|---|
| 99.1% | 98.7% | 98.0% | 97.4% | 97.0% |

In the example, formulation composition pH had a considerable effect on the radiochemical stability of ¹⁷⁷Lu-PSMA I&T and more specifically in the formation of radiochemical impurity eluting at about 5.2 minutes as exemplified in FIGS. 6A-11B. In high RAC solutions, the decrease in radiochemical purity over time at pH 4.5 was two times slower than at pH 7.

Further decrease of formulation pH to 3.5 did not show measurable improvement in radiochemical stability compared to pH 4.5 solution. It is possible that ascorbic acid solution at pH 4.5, being near pKa value of ascorbic acid, already possesses sufficient quantity of protons to serve as inhibitors against radiolysis of ¹⁷⁷Lu-PSMA I&T and reduce formation of the radiochemical impurity eluting at about 5.2 minutes.

Incorporation of solution pH of 4.5 in the lower RAC formulation further improved radiochemical stability of ¹⁷⁷Lu-PSMA I&T. In the lower RAC formulation, change in formulation pH from 5 to 4.5 had similar effect on radiochemical stability as increasing ascorbic acid concentration from 21 mg/ml to 31 mg/ml.

The radiochemical purity of a 10 ml high RAC ¹⁷⁷Lu-PSMA I&T formulation composition comprising 42.5 mg/ml ascorbic acid is at least about 99% at 0 hours post EOS and at least about 93.3% at 46 hours post EOS, as measured by HPLC. The radiochemical purity decreases as the pH of the formulation is increased from pH 4.5.

The radiochemical purity of a 20 ml low RAC ¹⁷⁷Lu-PSMA I&T formulation comprising 31 mg/ml ascorbic acid is at least about 99.1% at 0 hours post EOS as measured by HPLC. The rate in which the radiochemical purity decreases over time is slower for low RAC ¹⁷⁷Lu-PSMA-I&T formulations having a pH of 4.5 compared to a pH of 5.

The results demonstrate that, formulation composition pH of 5 or below can substantially reduce formation of radiochemical impurity eluting at about 5.2 minutes and therefore enhance radiochemical stability of ¹⁷⁷Lu-PSMA I&T compared to formulation compositions at pH above 5. In addition, radiochemical stability of ¹⁷⁷Lu-PSMA I&T can further be improved by incorporation of lower solution RAC. In the example, ¹⁷⁷Lu-PSMA I&T solution showed highest radiochemical stability in low RAC solution of pH 4.5 and ascorbic acid concentration of 31 mg/ml. This formulation is considered a preferred composition to minimize formation of radiochemical impurities and to maintain radiochemical stability of ¹⁷⁷Lu-PSMA I&T above 95.0% for 72 hours or longer.

### Example 4: Effects of Treatment with Both ¹⁷⁷Lu-PSMA-617 and ¹⁷⁷Lu-PSMA-I&T Compared to Third Line Treatment

### Dosimetry for ¹⁷⁷Lu-PSMA I&T

There was no major difference in absorbed dose estimates between ¹⁷⁷Lu-PSMA I&T and ¹⁷⁷Lu-PSMA-617. The specific known dosimetry for ¹⁷⁷Lu-PSMA I&T is presented below.

For normal organs, the mean whole-body effective dose for all cycles was 0.41±0.18 Sv (0.06 Sv/GBq). The mean absorbed organ doses were 5.3±1.6 Gy (0.72 Gy/GBq) for the kidneys; 0.89±0.42 Gy (0.12 Gy/GBq) for the liver; and 4.0±1.1 Gy (0.55 Gy/GBq) for the parotid, 4.8±2.8 Gy (0.64 Gy/GBq) for the submandibular, and 27±10 Gy (3.8 Gy/GBq) for the lacrimal glands.

No substantial difference for absorbed doses of normal organs were observed when comparing them with respect to cycle number (Table 13). The mean organ masses underlying these absorbed dose estimates were 1.595±307 g (range, 1,165-2,373 g) for the liver, 153±29.9 g (range, 88.4-218.7 g) for the kidneys, 19.1±5.7 g (range, 8.0-35.6 g) for the parotid, 8.2±1.9 g (range, 4.2-14.3 g) for the submandibular, and 0.45±0.12 g (range, 0.25-0.78 g) for the lacrimal glands. For paired organs, masses from both sides were summed.

For tumor lesions, all lesions received a mean dose per cycle of 23±20 Gy (3.3 Gy/GBq). Mean absorbed doses for bone, lymph node, liver, and lung metastases were 26±20 Gy (3.4 Gy/GBq), 24±16 Gy (3.2 Gy/GBq), 8.5±4.7 Gy (1.28 Gy/GBq), and 13±7.4 Gy (1.7 Gy/GBq).

The values (mean, SD, and ranges) for the corresponding absorbed doses per GBq for normal organs and tumor lesions are presented in the tables below, respectively.

TABLE 13
Effective Dose for Whole Body in Sv/GBq and Absorbed Doses for NormalOrgans in Gy/GBq.

| | | | | Parotid | Submandibular | Lacrimal |
|---|---|---|---|---|---|---|
| Cycles investigated | Whole body | Kidneys* | Liver | glands* | glands* | glands* |
| Overall (n = 34) | | | | | | |
| Mean ± SD | 0.06 ± 0.03 | 0.72 ± 0.21 | 0.12 ± 0.06 | 0.55 ± 0.14 | 0.64 ± 0.40 | 3.8 ± 1.4 |
| Range | 0.02-0.11 | 0.33-1.22 | 0.05-0.26 | 0.25-0.84 | 0.24-1.70 | 1.68-7.03 |
| First cycle (n = 15) | | | | | | |
| Mean ± SD | 0.05 ± 0.03 | 0.71 ± 0.25 | 0.12 ± 0.07 | 0.56 ± 0.17 | 0.54 ± 0.29 | 3.8 ± 1.5 |
| Range | 0.02-0.11 | 0.33-1.22 | 0.05-0.26 | 0.25-0.84 | 0.25-1.35 | 1.68-7.03 |
| Second cycle (n = 9) | | | | | | |
| Mean ± SD | 0.06 ± 0.02 | 0.75 ± 0.19 | 0.13 ± 0.05 | 0.59 ± 0.13 | 0.71 ± 0.42 | 4.1 ± 1.1 |
| Range | 0.03-0.11 | 0.44-1.01 | 0.08-0.21 | 0.47-0.80 | 0.40-1.70 | 2.44-5.56 |
| Third cycle (n = 5) | | | | | | |
| Mean ± SD | 0.06 ± 0.03 | 0.73 ± 0.22 | 0.12 ± 0.06 | 0.49 ± 0.11 | 0.83 ± 0.59 | 3.5 ± 1.4 |
| Range | 0.03-0.10 | 0.42-0.93 | 0.07-0.21 | 0.32-0.64 | 0.33-1.68 | 2.41-5.04 |
| Fourth cycle (n = 5) | | | | | | |
| Mean ± SD | 0.05 ± 0.03 | 0.69 ± 0.18 | 0.11 ± 0.04 | 0.50 ± 0.11 | 0.66 ± 0.53 | 3.4 ± 1.7 |
| Range | 0.03-0.11 | 0.45-0.95 | 0.07-0.17 | 0.32-0.58 | 0.24-1.58 | 1.75-5.35 |

TABLE 14
Absorbed Doses for Tumor Lesions in Gy/GBq

| | All | Bone | Lymph node | Liver | Lung |
|---|---|---|---|---|---|
| Cycles investigated | metastases | metastases | metastases | metastases | metastases |
| Overall (n) | 93 | 74 | 8 | 8 | 3 |
| Mean ± SD | 3.2 ± 2.6 | 3.4 ± 2.7 | 3.2 ± 2.2 | 1.2 ± 0.67 | 1.75 ± 0.92 |
| Range | 0.22-12.03 | 0.22-12.03 | 1.63-8.46 | 0.47-2.59 | 0.94-2.68 |
| First cycle (n) | 41 | 33 | 5 | 2 | 1 |
| Mean ± SD | 3.5 ± 2.9 | 3.8 ± 3.1 | 2.6 ± 0.89 | 1.7 | 2.7 |
| Range | 0.22-12.03 | 0.22-12.03 | 1.63-3.76 | 0.85-2.59 | |
| Second cycle (n) | 26 | 21 | 2 | 2 | 1 |
| Mean ± SD | 3.3 ± 2.5 | 3.4 ± 2.4 | 5.2 | 0.94 | 1.3 |
| Range | 0.70-8.46 | 1.03-9.59 | 1.98-8.46 | 0.70-1.17 | |
| Third cycle (n) | 14 | 10 | 1 | 2 | 1 |
| Mean ± SD | 2.7 ± 2.3 | 3.2 ± 2.5 | 2.6 | 0.95 | 0.94 |
| Range | 0.94-7.99 | 1.11-7.99 | 18.87 | 0.47-1.42 | |
| Fourth cycle (n) | 12 | 10 | | 2 | |
| Mean ± SD | 2.4 ± 2.2 | 2.7 ± 2.3 | | 1.13 | |
| Range | 0.74-7.60 | 1.04-7.60 | | 0.74-1.51 | |

There is a clear trend toward a lower absorbed dose with an increasing number of the cycle. Mean absorbed dose per lesion was 26±21 Gy (3.5 Gy/GBq) for the first, 24±19 Gy (3.3 Gy/GBq) for the second, 20±18 Gy (2.7 Gy/GBq) for the third, and 18±17 Gy (2.4 Gy/GBq) for the fourth cycle. A similar trend can be seen for the subgroup of bone metastases. No reliable comparison is possible for lymph node, liver, and lung metastases because of a low sample number. See the below table for the effective half-lives and mean absorbed doses of ¹⁷⁷Lu-PSMA I&T.

¹⁷⁷Lu-PSMA RLT, the study included both ¹⁷⁷Lu-PSMA-617 and ¹⁷⁷Lu-PSMA I&T, had better effects and caused less adverse effects than third-line treatment. Twelve studies including 669 patients reported ¹⁷⁷Lu-PSMA RLT. Overall, 44% of the patients had a maximum decline of PSA of >50% following treatment with ¹⁷⁷Lu-PSMA RLT. The treatment with ¹⁷⁷Lu-PSMA-617 and ¹⁷⁷Lu-PSMA for imaging and therapy (I&T) had mainly transient adverse effects. Sixteen studies including 1338 patients reported third-line treatment. Overall, 21% of the patients had a best decline of PSA of >50% following third-line treatment. After third-line treatment with enzalutamide and cabazitaxel, adverse effects caused discontinuation of treatment for 10% to 23% of the patients. ¹⁷⁷Lu-PSMA RLT gave a best PSA decline >50% more often than third-line treatment (mean 44% versus 22%, p=0.0002, t-test). ¹⁷⁷Lu-PSMA RLT gave objective remission more often than third-line treatment (overall 31 of 109 patients versus 43 of 275 patients, p=0.004, X2 test). Median survival was longer after ¹⁷⁷Lu-PSMA RLT than after third-line treatment, but the difference was not statistically significant (mean 14 months versus 12 months, p=0.32, t-test). Adverse effects caused discontinuation of treatment more often for third-line treatment than for ¹⁷⁷Lu-PSMA RLT (22 of 66 patients versus 0 of 469 patients, p<0.001, χ2 test).

The aim of this investigational medical product dossier (IMPD) is to provide a scientific and ethical platform for this useful treatment with ¹⁷⁷Lu-PSMA I&T to initially pursue its application, preferably in academic centers and under controlled investigational protocols. There are not existing guidelines except for ¹⁷⁷Lu-PSMA-617. The information in this IMPD is based on the newest literature and existing best experience from the nuclear medicine centers who have been treating PC patients with this modality.

The synthesis is a one-step labelling process with injections grade ethanol and water used as the only solvents. Therefore, no residual solvents are present. Radiochemical impurities are quantified by chromatographical methods (HPLC and TLC). Radiochemical purity may not be less than 95.0%.

### Example 5: A Multi-Center, Open-Label, Randomized Phase 3 Trial Comparing the Safety and Efficacy of ¹⁷⁷Lu-PSMA I&T

A Multi-Center, Open-Label, Randomized Phase 3 Trial was conducted comparing the safety and efficacy of the composition comprising ¹⁷⁷Lu-PSMA I&T versus hormone therapy in patients with Metastatic Castration-Resistant Prostate Cancer.

This study aims to identify and characterize the safety and efficacy of using ¹⁷⁷Lu-PSMA I&T in the treatment of adult male human patients (men) with metastatic Castration-Resistant Prostate Cancer (mCRPC) who progress despite treatment with one course of standard of care hormone therapy.

¹⁷⁷Lu-PSMA I&T is a radioactive therapeutic agent that specifically targets the prostate specific membrane antigen proteins that are expressed on metastatic prostate cancer cells.

In this study, ¹⁷⁷Lu-PSMA I&T is provided as a sterile, filtered radiopharmaceutical solution that contains a microdose of ¹⁷⁷Lu-PSMA I&T in an aqueous ascorbic acid and ethanol solution.

Patients randomized to be treated in accordance with the standard of care hormone therapy for mCRPC will be treated either with abiraterone acetate in combination with prednisone, or enzalutamide based on the Investigator's independent medical judgment.

Abiraterone acetate is indicated in combination with prednisone for the treatment of patients with metastatic castration-resistant prostate cancer (mCRPC) or metastatic high-risk castration-sensitive prostate cancer (mCSPC). Abiraterone acetate is converted in vivo to abiraterone, an androgen biosynthesis inhibitor, that inhibits 17 α-hydroxylase/C17,20-lyase (CYP17). This enzyme is expressed in testicular, adrenal, and prostatic tumor tissues and is required for androgen biosynthesis.

Enzalutamide is an androgen receptor inhibitor indicated for the treatment of patients with CRPC or mCSPC (Metastatic Castration-Sensitive Prostate Cancer). Enzalutamide has been shown to competitively inhibit androgen binding to androgen receptors and, consequently, inhibits nuclear translocation of androgen receptors and their interaction with DNA.

### A. Study Objectives and Endpoints

The primary objective of this study is to prospectively assess the efficacy of ¹⁷⁷Lu-PSMA I&T on the improvement of radiographic progression-free survival (rPFS) as determined by PCWG3-modified RECIST 1.1 in men with metastatic Castration-Resistant Prostate Cancer (mCRPC) compared to standard of care hormone therapy. The endpoint of this objective is the time from randomization to radiographic progression as determined by Prostate Cancer Working Group 3 (PCWG3) criteria as assessed by blinded independent central review.

The secondary objective is to assess if ¹⁷⁷Lu-PSMA I&T improves overall survival (OS) in patients with mCRPC compared to those treated with standard of care hormone therapy. The endpoint of this objective is the time from randomization to death by any cause.

Other secondary objectives and endpoints include:
Objective: evaluate the improvement in overall survival (OS) in men with mCRPC treated with ¹⁷⁷Lu-PSMA I&T compared to hormone therapy; Endpoint: time from randomization to the second radiographic progression as determined by PCWG3 or RECIST 1.1 by BICR (Blinded Independent Central Review) after crossover.
Objective: assess changes in the time to second radiographic progression for patients who crossover from the standard of care hormone therapy arm to the ¹⁷⁷Lu-PSMA I&T treatment arm; Endpoint: time from randomization to the second radiographic progression as determined by PCWG3 or RECIST 1.1 by BICR after crossover,
Objective: identify changes in progression-free survival (PFS, composite) following ¹⁷⁷Lu-PSMA I&T radioligand therapy compared to standard of care hormone therapy; Endpoint: time from randomization to progression (PFS, composite) based on the following events, whichever occurs first: PCWG3 or RECIST progression, clinical/symptomatic progression and/or pain progression, or death due to any cause as determined by investigator.
Objective: identify changes in progression-free survival 2 (PFS2, composite) following ¹⁷⁷Lu-PSMA I&T radioligand therapy compared to standard of care hormone therapy; Endpoint: time from randomization to the second progression (PFS, composite) based on the following events, whichever occurs first: PCWG3 or RECIST progression, clinical/symptomatic progression and/or pain progression, or death due to any cause as determined by investigator,
Objective: assess changes in PSA50 (Response Rate of Patients with a reduction of PSA 50% from Baseline) response rate following ¹⁷⁷Lu-PSMA I&T radioligand therapy compared to standard of care hormone therapy; Endpoint: PSA50 response rate, defined as a confirmed reduction of PSA from baseline of 50%,
Objective: determine the impact of ¹⁷⁷Lu-PSMA I&T compared to standard of care hormone therapy on skeletal symptoms; Endpoint: time from randomization to first symptomatic skeletal event (SSE-free survival),
Objective: determine the impact of ¹⁷⁷Lu-PSMA I&T compared to standard of care hormone therapy on radiographic soft-tissue progression; Endpoint: time from randomization to radiographic soft tissue progression (rSTP) as measured by RECIST 1.1 by BICR,
Objective: assess changes in use of chemotherapy following ¹⁷⁷Lu-PSMA I&T compared to standard of care hormone therapy; Endpoint: time from randomization to first use of chemotherapy, and
Objective: evaluate the impact on quality-of-life following ¹⁷⁷Lu-PSMA I&T radioligand therapy to hormone therapy; Endpoint: Quality of Life improvement based on EORTC QLQ-C30 questionnaire.

Exploratory objectives and endpoints are:
Objective: evaluate any differences in objective response rate and disease control rate with ¹⁷⁷Lu-PSMA I&T compared to standard of care hormone therapy; Endpoint: objective response rate based on PCWG3 criteria and disease control rate (DCR=complete/partial response and stable disease),
Objective: evaluate changes in time to PSA progression following ¹⁷⁷Lu-PSMA I&T radioligand therapy compared to standard of care; Endpoint: time from randomization to PSA progression, defined as a rise in PSA of ≥25% from the post-treatment nadir, and
Objective: assess the duration of response (DoR) in patients who achieve complete or partial response following ¹⁷⁷Lu-PSMA I&T radioligand therapy compared to hormone therapy; Endpoint: the time from complete or partial response to radiographic progression.
   B. study design

(i) Overview and Rationale

This is an open-label, randomized, multicenter Phase 3 study of ¹⁷⁷Lu-PSMA I&T radioligand therapy compared to hormone therapy in men with mCRPC who have been previously treated with androgen receptor (AR)-directed therapy. The hormone therapy regimens for this study are enzalutamide or abiraterone with prednisone based on NCCN guidelines. Selection of the specific regimen will be based on a switch from the patient's previous ADRT for patients randomized to receive standard of care. Based on the published literature, ¹⁷⁷Lu-PSMA I&T radioligand therapy has encouraging anti-tumor activity in men with mCRPC and is associated with a favorable safety profile.

The study consists of a Screening Phase, a Treatment Phase, and a Post-Treatment Follow-up Phase. The study employs a 2:1 randomization into the following treatment groups: (1)¹⁷⁷Lu-PSMA I&T radioligand therapy, or (2) Standard of Care hormone therapy. Standard of care hormone treatment options are abiraterone with prednisone or enzalutamide, with the specific choice based on the Investigator's clinical judgment. Patients randomized to the hormone therapy arm will be given the option to crossover to the radioligand therapy arm upon documentation of radiographic progression.

Patients will be followed for safety and efficacy as per the schedule of activities and will remain on study treatment until documented radiographic progression occurs as assessed by a blinded independent central review (BICR) or the development of unacceptable toxicity. Patients discontinuing treatment due to documented radiographic progression will enter the Long-term Follow-up Phase. Patients discontinuing treatment prior to documented radiographic progression will continue to have scheduled disease assessments every three months until documented radiographic progression.

In addition, a sub-study will be conducted under this protocol in patients randomized to receive ¹⁷⁷Lu-PSMA I&T radioligand therapy to evaluate the pharmacokinetic and radiation dosimetry (as discussed in Example 6).

Due to the nature of the treatments, the identity of the test and control treatments will be known to the Investigators, research staff, and patients. Blinding of this study is not feasible. Immediately following Investigator determination of progressive disease for progressing patients and following the completion of the study for non-progressing patients, blinded radiographic images will be read and interpreted by a panel of up to three trained independent radiologists without access to clinical information or treatment groups for assessing the overall response rate to treatment.

The severity of AEs and SAEs will be graded based upon the subject's symptoms in accordance with the Common Terminology Criteria for Adverse Events (CTCAE), Version 5.0. AEs that are not defined in the CTCAE current version should be evaluated for severity in accordance with the following scale:
Grade 1=Mild: transient or mild discomfort, no limitation in activity, no medical intervention/therapy required
Grade 2=Moderate: mild to moderate limitation in activity, some assistance may be needed, no or minimal medical intervention/therapy required
Grade 3=Severe: marked limitation in activity, some assistance usually required, medical intervention/therapy required, hospitalization possible
Grade 4=Life-threatening: extreme limitation in activity, significant assistance required, significant medical intervention/therapy required, hospitalization or hospice care probable
Grade 5=Death: the event results in death

It is important to distinguish between serious and severe AEs. Severity is a measure of intensity, whereas seriousness is defined by the criteria outlined in Section 10.3. An AE of severe intensity may not be considered serious. Seriousness, not severity, serves as a guide for defining regulatory obligations.

(ii) Selection of the Primary Endpoint

Metastatic castration-resistant prostate cancer is generally considered a late stage in the natural progression of prostate cancer. Although mCRPC is usually associated with an unfavorable prognosis, many patients experience a more indolent disease progression resulting in a larger range of overall survival with approximately 15% of men with mCRPC surviving beyond 5 years (Moreira et al., Clin Genitourin Cancer, 2017; 15(1): 60-66).

In consideration of the relatively large range of overall survival in men with mCRPC, radiographic progression-free survival using RECIST 1.1 (soft tissue lesion status), and Prostate Cancer Working Group 3 (PCWG3) criteria (bone lesion status), as assessed by blinded independent central review, was chosen as the primary endpoint for this study. Overall survival is a secondary endpoint of the study, and patients will be followed for overall survival for a period of 5 years after enrollment.

(iii) Study Outcomes

The primary efficacy outcome is radiographic progression-free survival. Once radiographic progression is confirmed by the BIRC, study treatments will be discontinued. Patients will then enter the Follow-up Phase of the trial to assess overall survival for a 5-year follow-up period from the date of enrollment in the study.

Safety will be assessed until the end of treatment and one-month follow-up period by assessing the following safety parameters: adverse events, vital signs, changes in concomitant medications/therapies, changes in physical examination, and clinical laboratory measurements.

C. patient selection

### (i) Study Population

The study population will include patients with mCRPC with progressive disease based on PCWG3 modified RECIST 1.1 criteria.

(ii) Inclusion Criteria Include:
1. Male 18 years or older.
2. Histologically or pathologically confirmed prostate adenocarcinoma without predominant small cell component.
3. Progressive disease by one or more of the following criteria:
   a. Serum/plasma PSA progression defined as 2 consecutive increases in PSA over a previous reference value measured at least 1 week apart with a minimum start value of >2 ng/mL.
   b. Progression of measurable disease (RECIST 1.1) or presence of at least two new bone lesions (PCWG3 criteria)
4. Previous treatment with next-generation androgen receptor (AR)-directed therapy (e.g. abiraterone, enzalutamide, apalutamide, darolutamide).
   a. Must have received no more than one previous AR-directed therapy.
   b. Must have been administered ARAT (abiraterone, enzalutamide, darolutamide, or apalutamide) in the castration-sensitive or castration-resistant setting.
   c. Must have progressed while on ARAT.
5. PSMA-PET scan (e.g., [⁶⁸Ga]Ga-PSMA-11 or [¹⁸F]DCFPyL) positive as determined by central reader.
6. Effective castration with serum testosterone level of <50 ng/dL and plan to continue with chronic medical or surgical castration.
7. Patients with HIV that are healthy and with a low risk of acquired immune deficiency syndrome related outcomes may participate in the study at the investigators' discretion.
8. Patients with HBV and HCV may also participate if symptoms are sufficiently managed.
9. Life expectancy of at least 6 months as assessed by investigator.
10. Willing to initiate ARAT therapy determined by investigator.

(iii) Exclusion Criteria Include:
1. Prior treatment with radioligand therapy including other lutetium-labeled compounds.
2. Prior treatment with radium-223 (Xofigo) within the past 12 weeks.
3. Prior chemotherapy treatment for castration-sensitive or castration-resistant prostate cancer (docetaxel or cabazitaxel).
4. Eastern Cooperative Oncology Group (ECOG) performance status (PS) ≥2.
5. Patients with known HRR (Haploid relative risk) gene-mutation who have not been previously treated with olaparib or rucaparib.
6. Other concurrent cytotoxic chemotherapy, immunotherapy, radioligand therapy, or investigational therapy.
7. Inadequate organ and bone marrow function as evidenced by:
   a. Hemoglobin <8 g/dL.
   b. Absolute neutrophil count <1.5×109/L.
   c. Platelet count <100×109/L.
   d. AST/SGOT and/or ALT/SGPT >3.0×ULN (wherein: “AST” is Aspartate aminotransferase, “SGOT” is Serum glutamic-oxaloacetic transaminase, “SGPT” is Serum glutamic-pyruvic transaminase, and “ALT” is Alanine aminotransferase).
   e. Total bilirubin >2×ULN (Upper Limit of Normal) unless patient has known Gilbert's syndrome and then may be 3×ULN.
   f. Creatinine clearance (CrCl)<50 mL/min based on the Cockcroft-Gault equation.
   g. Albumin 2.75 g/dL
8. Patients who undergo a transfusion for the sole purpose of meeting eligibility for this study.
9. Use of an investigational therapeutic drug within the last 4 weeks prior to start of study treatment or scheduled to receive one during the study period.
10. Known CNS metastasis unless received therapy, asymptomatic and neurologically stable.
11. Patients receiving zoledronic acid for bone-targeted therapy must be on stable dose for 4 weeks prior to randomization.
12. Patients with active significant cardiac disease 13. Participants with symptomatic cord compression or clinical/radiological findings indicating impending spinal cord compression.
14. Patients with a superscan seen on baseline bone scan as determined by investigator.
15. Active malignancy other than low-grade non-muscle-invasive bladder cancer and non-melanoma skin cancer.
16. Previous use of G-CSF (Granulocyte-colony stimulating factor) for persistent neutropenia after standard of care treatment.
17. Participants with active Covid19. Recovered patients may be included when completely recovered (no symptoms at least 28 days before study medication and a negative Covid test within 72 hours).

### D. Investigational Product Dosage, Route of Administration, and Dosing Schedule

(i)¹⁷⁷Lu-PSMA I&T

The medicinal product is a sterile filtered radiopharmaceutical solution that contains a microdose of ¹⁷⁷Lu-PSMA I&T formulated in an aqueous solution containing ascorbic acid and ethanol. The product contains a standard activity of approximately 200 mCi at the time of expiry with a standard concentration of approximately 27 mCi/mL at end of production; therefore, the final volume of the dose vial is adjusted to between 7.0 and 10.0 mL in order to provide the required amount of radioactivity at the date and time of infusion. ¹⁷⁷Lu-PSMA I&T injection is supplied as a sterile solution in a single-dose vial. The septum is sealed with a crimped aluminum cap. The glass vial containing the radiopharmaceutical is kept in a lead shielded container until use. ¹⁷⁷Lu-PSMA I&T is stored at 25° C.; excursions between 15° C.-30° C. are permitted. The shield label will state the expiration date and time for each vial shipped to the clinical sites.

(ii) Standard of Care Hormone Treatments

Standard of care hormone treatments are:
Abiraterone Acetate with Prednisone: Abiraterone acetate is a CYP 17 inhibitor used in combination with prednisone or methylprednisone. Prednisone is a glucocorticoid. Glucocorticoids are adrenocortical steroids that are readily absorbed from the gastrointestinal tract.
Enzalutamide is an androgen receptor inhibitor.

(iii) Preparation

All infusion solutions will be prepared and dispensed by the site prior to administration. The preparation of solutions should be performed under aseptic conditions, and final solutions should be inspected visually for particulate matter. If an insoluble precipitate is observed, the solution should be discarded.

¹⁷⁷Lu-PSMA I&T injection solution is administered as supplied. The radioactivity in the vial should be measured in a calibrated radiation dose calibrator prior to, and after administration to the patient. The administered dose should then be calculated and recorded.

(iv) Dosing and Administration

Patients will be randomized 2:1 to receive either ¹⁷⁷Lu-PSMA I&T radioligand therapy or standard of care hormone therapy.

¹⁷⁷Lu-PSMA I&T Administration: Patients randomized to receive radioligand therapy will receive a single intravenous radioactive dose of 200 mCi (7.4 GBq)±10% of ¹⁷⁷Lu-PSMA I&T at the beginning of each treatment cycle.

An intravenous line should be established prior to administration of ¹⁷⁷Lu-PSMA I&T. The ¹⁷⁷Lu-PSMA I&T will be injected as a slow bolus over a minimum of 10 to 15 minutes using the site's standard radioligand therapy administration procedures. The patient should be encouraged to void as frequently as possible and drink two liters of liquid daily for two days following ¹⁷⁷Lu-PSMA I&T administration.

Cooling of the patients' salivary glands should be performed by placing ice packs over the parotid and submandibular glands for 30 minutes prior to and up to 4 hours after the injection of ¹⁷⁷Lu-PSMA I&T to reduce the risk of salivary gland radiation injuries.

A six-week treatment cycle will be used for injection of ¹⁷⁷Lu-PSMA I&T 4 treatment cycles or until radiographic progression of disease is determined based on the BIRC assessment of radiographic images (maximum 18 weeks of treatment). Alternatively an eight-week treatment cycle may be used for injection of ¹⁷⁷Lu-PSMA I&T 6 treatment cycles or until radiographic progression of disease is determined based on the BIRC assessment of radiographic images (maximum 18 weeks of treatment).

The dosing cycle for ¹⁷⁷Lu-PSMA I&T may be extended based on evaluation of dose-limiting toxicity experienced by a patient. Dose-limiting toxicity is defined as grade 3 or 4 bone marrow toxicity or grade 2 or greater salivary gland toxicity. For grade 3 or 4 bone marrow toxicity, dosing may resume when improvement to grade 2 or better is observed. For grade 2 or greater salivary gland toxicity, dosing may resume upon improvement to grade 1 toxicity.

In addition, the ¹⁷⁷Lu-PSMA I&T dose should be held and/or reduced to 160 mCi (5.9 GBq)±10% if dose-limiting toxicity is noted. The following dose hold/reductions should be implemented accordingly:
For grade ≥3 Anemia: Hold dose until return to baseline or Grade ≤2 and then reduce to 160 mCi (5.9 GBq) for next cycle
For grade ≥2 Neutropenia: Hold until return to baseline or Grade ≤1
For grade ≥2 Thrombocytopenia: Hold until return to baseline or Grade ≤1 and reduce dose to 160 mCi (5.9 GBq) for next cycle
For grade ≥3 Non-platelet hematological toxicity: Hold until return to baseline or Grade ≤2 and reduce dose to 160 mCi (5.9 GBq) for the next cycle

For cases of acute renal toxicity that is Grade 3 or higher, dose should be held for next cycle. Dose may be reinstated upon return to baseline or Grade 52 but reduced to 160 mCi (5.9 GBq) for all remaining doses.

Dosing cycle extensions of an additional six weeks and dose reduction modifications to 160 mCi (5.9 GBq) for remaining cycles should be initiated for any other Grade 3 or higher non-hematological toxicity related to ¹⁷⁷Lu-PSMA I&T as determined by the Investigator. Dosing cycle and dose level may be returned to every six weeks when toxicity returns to a Grade 2 or below.

For all grade 3 and 4 AEs, patients will only be able to have a 1-time reduction in dose. If the event persists, the patient would need to permanently discontinue from study treatment.

For grade 2 AEs, only 2 dose reductions are permitted. If the event persists the patient would need to permanently discontinue from study treatment.

Abiraterone Acetate with Prednisone: The dose of abiraterone acetate should be administered per package insert. Per Abiraterone package insert, for patients with baseline moderate hepatic impairment, the starting dose of abiraterone should be reduced to 250 mg daily. For patients who develop hepatoxicity during treatment, dosing with abiraterone acetate should be suspended until recovery. Retreatment may be initiated at a reduced dose. Abiraterone acetate treatment should be discontinued in patients that develop severe hepatotoxicity.

Enzalutamide: The dose of enzalutamide is 160 mg (four 40 mg capsules) administered orally once daily. Capsules should be swallowed whole and can be taken with or without food. Per enzalutamide package insert, if a patient experiences a ≥Grade 3 toxicity or an intolerable side effect, dosing should be withheld for one week or until symptoms improve to ≤Grade 2, then resumed at the same or a reduced dose (120 or 80 mg) if warranted.

(v) Duration of Study Treatment

Patients will be treated until radiographic progressive disease, clinical/symptomatic progression, unacceptable/unmanageable toxicity, or patient's refusal of further study treatment (i.e., withdrawal of consent). All patients will be followed while on study treatment and after completion of study treatment during follow-up period: until death, the study cut-off date (which is a minimum of 22 weeks post-enrollment), or withdrawal of consent, whichever comes first. Long-term follow-up with all patients will be five years from enrollment or until death or loss to follow-up.

### E. Study Procedures and Evaluations

Trial evaluations and time points are summarized in the Schedule of Activities in Tables 15 and 16.

TABLE 15
Schedule of Events for ¹⁷⁷Lu-PSMA-I&T Arm.

| | | Treatment Phase | | | | | | | | Post-treatment Phase | | |
|---|---|---|---|---|---|---|---|---|---|---|---|---|
| | Screening | Cycle 1 | | Cycle 2 | | Cycle 3 | | Cycle 4 | | EOT | LTFU | |
| Week (±7 days) | −4 to 0 | 0 | 4 | 6 | 8 | 12 | 16 | 18 | 20 | 22 | 24-256 | US |
| Informed Consent | X | | | | | | | | | | | |
| Demographics | X | | | | | | | | | | | |
| Inclusion/Exclusion | X | | | | | | | | | | | |
| Criteria | | | | | | | | | | | | |
| Medical History | X | | | | | | | | | | | |
| PSMA PET | X | | | | | | | | | | | |
| CT and Bone Scan^{a} | X | | | | X | | X | | | | X^{a} | |
| ¹⁷⁷Lu-PSMA-I&T | | X | | X | | X | | X | | | | |
| Treatment | | | | | | | | | | | | |
| Hematology | X | X | X | | X | X | X | | X | X | | |
| Blood Chemistry | X | X | X | | X | X | X | | X | X | | |
| PSA | X | X | | | X | | X | | | X | | |
| ECOG | X | X | | | | | | | | | | |
| Physical Exam | X | X | X | | | | | | | X | | |
| Vital Signs | X | X | X | | | | | | | X | | |
| 12-Lead ECG | X | X | | | | | | | | | | |
| Concomitant | X | X | X | X | X | X | X | X | X | X | X | |
| Medications | | | | | | | | | | | | |
| EORTC | | X | X | | X | X | X | | X | X | | |
| FACT-P | | X | X | | X | X | X | | X | X | | |
| BPI-SF | | X | X | | X | X | X | | X | X | | |
| Adverse Events | X | X | X | X | X | X | X | X | X | X | X | X |
| Progression^{b} | | | | | X | | X | | X | | X | |
| Survival | | | | | | | | | | | X | |

EOT = End of Treatment;
LTFU = Long-Term Follow Up;
US = Unscheduled
^{a}CT/Bone scan will be performed every 8 weeks throughout treatment until week 24, and then, if no progression, every 12 weeks continued in LTFU.
^{b}Per section 8.5, progression assessments to be conducted every 12 weeks throughout LTFU until radiographic evidence of disease progression is noted

TABLE 16
Schedule of Events for Standard of Care Hormone Therapy Arm.

| | Screening | Treatment Phase | | | | | | EOT | LTFU | |
|---|---|---|---|---|---|---|---|---|---|---|
| Week (±7 days) | −4 to 0 | 0 | 4 | 8 | 12 | 16 | 20 | 22 | 24-256 | US |
| Informed Consent | X | | | | | | | | | |
| Demographics | X | | | | | | | | | |
| Inclusion/Exclusion | X | | | | | | | | | |
| Criteria | | | | | | | | | | |
| Medical History | X | | | | | | | | | |
| PSMA PET | X | | | | | | | | | |
| CT and Bone Scan^{a} | X | | | X | | X | | | X^{a} | |
| Abiraterone or | | Daily | | | | | | | | |
| Enzalutamide | | | | | | | | | | |
| Treatment | | | | | | | | | | |
| Hematology | X | X | X | X | X | X | X | X | | |
| Blood Chemistry | X | X | X | X | X | X | X | X | | |
| PSA | X | X | | X | | X | | X | | |
| ECOG | X | X | | | | | | | | |
| Physical Exam | X | X | X | | | | | X | | |
| Vital Signs | X | X | X | | | | | X | | |
| 12-Lead ECG | X | | | | | | | | | |
| Concomitant | X | X | X | X | X | X | X | X | X | |
| Medications | | | | | | | | | | |
| EORTC | | X | X | X | X | X | X | X | | |
| FACT-P | | X | X | X | X | X | X | X | | |
| BPI-SF | | X | X | X | X | X | X | X | | |
| Adverse Events | X | X | X | X | X | X | X | X | X | X |
| Progression^{b} | | | | X | | X | X | | X | |
| Survival | | | | | | | | | X | |

EOT = End of Treatment;
LTFU = Long-Term Follow Up;
US = Unscheduled
^{a}CT/Bone scan will be performed every 8 weeks throughout treatment until week 24, and then, if no progression, every 12 weeks continued through LTFU.
^{b}Progression assessments to be conducted every 12 weeks throughout LTFU until radiographic evidence of disease progression is noted

(i) Clinical Assessments

Clinical assessments include demographics, medical history, physical examination, vital signs, performance status, adverse events, concomitant medications/therapies, tumor assessments, and Blinded Independent Central Review (BICR).

For performance status, the Eastern Cooperative Oncology Group (ECOG) performance status scale will be used and will be assessed at Screening and every subsequent clinic visit as shown below in Table 17:

TABLE 17
Eastern Cooperative Oncology Group
(ECOG) performance status scale/

| Grade | Activity Level |
|---|---|
| 0 | Fully active, able to carry on all pre-disease performance |
| | without restriction. |
| 1 | Restricted in physically strenuous activity but ambulatory |
| | and able to carry out work of light or sedentary nature (e.g., |
| | light housework, office work). |
| 2 | Ambulatory and capable of all self-care but unable to carry |
| | out any work activities, up and about more than 50% of waking |
| | hours. |
| 3 | Capable of only limited self-care, confined to bed or chair |
| | more than 50% of waking hours. |
| 4 | Completely disabled cannot carry on any self-care, totally |
| | confined to bed or chair. |
| 5 | Dead |

Tumor assessments will be performed according to the assessment calendar, regardless of treatment delays resulting from toxicity. Care must be taken in scheduling tumor assessments to prevent the introduction of bias based on treatment delays.

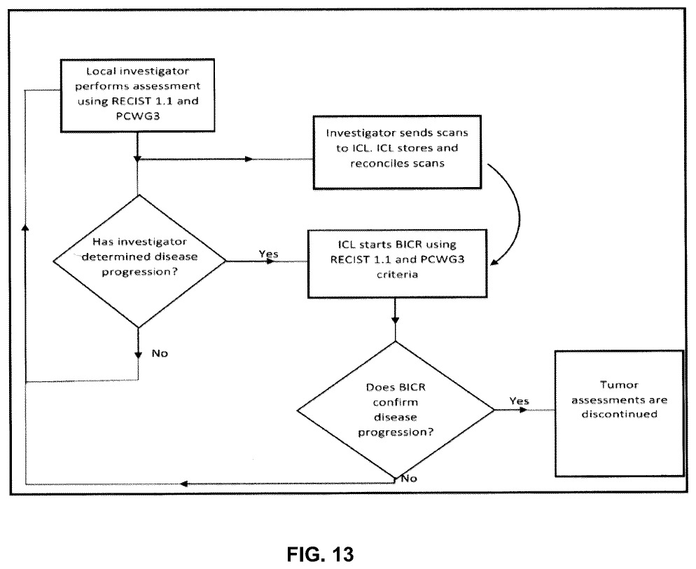

Patients will be evaluated for tumor response by CT imaging plus a bone scan at Screening and every 8 weeks (±1 week) following the start of treatment through week 24 of the study. Thereafter in patients without radiographic progression noted during the 24-week period, CT imaging and bone scans will be performed every 12 weeks (±1 week) until radiographic progression is determined. The schedule for scans will be based on calendar and not the start of a treatment cycle. Evaluations will include CT scans of chest, abdomen, pelvis, and brain (only as clinically warranted based on symptoms/findings). The Investigator, sub-Investigator, or qualified Site personnel will read the bone scans and CT images to assess whether radiographic progression has been noted. If the Investigator determines a patient's metastatic prostate cancer has progressed, the bone scans and CT images for the patient will immediately be transmitted to the Imaging Core Laboratory (ICL) for review by the Blinded Independent Central Review (BICR) to confirm radiographic progression as shown in FIG. 13.

No change in treatment should be made by the Investigator until after confirmation of disease status is received from the BICR.

Blinded Independent Central Review (BICR)
Screening: All scans (CT, bone, and PSMA PET), will be submitted to a third-party imaging core lab (ICL) for independent review of patient eligibility (within 3 days of receipt of imaging scans that pass quality assessment). Following confirmation by the BICR, the patient can be randomized into the study provided all other eligibility criteria have been met.
Radiographic Disease Progression: The investigator will assess the CT and bone scans to assess disease progression based upon RECIST 1.1 and PCWG3 criteria. If the investigator determines the disease progression has occurred, the BIRC, consisting of a panel two independent radiologists qualified to assess bone scans and CT images, will independently assess disease progression in accordance with RECIST 1.1 and PCWG3 criteria. Confirmation of radiographic disease progression requires agreement between the two blinded readers. If agreement between the two readers does not occur, a third reader will be utilized to adjudicate. The BICR will complete the confirmation of disease progression whenever possible within 72 hours of receipt of the image set from the Investigator demonstrative of radiographic progression. No alteration in the clinical management of the patient should be initiated by the Investigator prior to receipt of the BICR confirmation of radiographic progression. FIG. 13 presents the baseline and on-treatment disease status evaluation and treatment decisions.

(ii) Patient-Reported Outcomes

Patient-reported outcomes will be determined using the European Organization for Research and Treatment of Cancer Quality of Life Questionnaire (EORTC QLQ-C30), the Functional Assessment of Cancer Therapy-Prostate (FACT-P) questionnaire, and the Brief Pain Inventory-Short Form (BPI-SF) questionnaire.

The EORTC QLQ-C30 is a questionnaire of thirty quality of life (QoL) questions developed to assess the QoL of cancer patients. The QoL questionnaire has been included in over 3,000 Phase 3 cancer clinical trials as an efficacy endpoint. The EORTC questionnaire will be administered to the patients at baseline, during treatment as provided in the Schedule of Events in Table 15, and at the end of treatment.

The Functional Assessment of Cancer Therapy-Prostate (FACT-P) is a health-related Quality of Life questionnaire with 39 prostate cancer-specific questions that evaluates: Physical Well-Being, Functional Well-Being, Emotional Well-Being, Social Well-Being, and Additional Concerns or Prostate Cancer Subscale specific to prostate cancer. Higher FACT-P scores correspond to better quality of life. The FACT-P questionnaire will be administered to the patients at baseline, during treatment, and at the end of treatment.

The Brief Pain Inventory-Short Form (BPI-SF) is a short survey to assess overall pain and symptoms experienced by the participant. The BPI-SF questionnaire will be administered to the patients at baseline, during treatment, and at the end of treatment.

### F. Study Assessments by Visit

(i) Screening

Screening must be completed within 28 days prior to randomization into the study. The screening visit includes (a) drawing blood samples and submit to central laboratory for determining PSA level and baseline clinical laboratory assessments, (b) obtaining CT of the brain, chest, abdomen, and pelvis plus bone scan and submit to BICR within 72 hours to confirm patient eligibility, (c) obtaining PSMA-PET scan with FDA-approved radiotracer ([⁶⁸Ga]Ga-PSMA-11 or [¹⁸F]DCFPyL). PSMA-PET positivity is required for inclusion in the trial. PSMA-PET positivity is defined as PSMA-PET uptake greater than that of liver in one or more metastatic lesions of any size in any organ system, (d) recording medical history, including history of prostate cancer, diagnosis date, and prior treatments, (e) recording concomitant medications, (f) performing a complete physical examination, (g) performing and record vital signs and ECOG performance status grade, (h) performing 12-lead ECG, and (i) if patient is confirmed eligible by the Medical Monitor for enrollment in the study, randomizing patient in accordance with IVRS system and proceed to Cycle 1, Day 1 visit.

(ii) Treatment Day 1

The following assessments will be conducted on Treatment Day 1:
administering EORTC QLQ-C30, FACT-P, and BPI-SF questionnaires prior to treatment for baseline determination.
recording any changes to concomitant medications, and any adverse events noted from time of screening.
performing abbreviated physical examination
performing and recording vital signs and ECOG performance status.
collecting blood for clinical laboratory assessments.
administering study drug in the clinic. For patients enrolled into the standard of care arm of the study, initiate the Investigator's assigned standard of care hormone treatment:
   Abiraterone acetate with prednisone: The initial dose for abiraterone acetate is 1000 mg (four 250 mg tablets) administered once daily. The first dose of abiraterone acetate in combination with prednisone will be administered at the clinic. The time and date of the initiation of abiraterone therapy will be recorded.
   Enzalutamide: The initial dose for enzalutamide is 160 mg (four 40 mg capsules) administered once daily. The first dose of enzalutamide will be administered at the clinic. The time and date of the initiation of enzalutamide therapy will be recorded.
Patients enrolled in the radioligand therapy arm should be administered an initial radioactive dose of 200 mCi (7.4 GBq) of ¹⁷⁷Lu-PSMA-I&T infused over a minimum of 10 to 15 minutes. The dose and time period for ¹⁷⁷Lu-PSMA-I&T infusion will be recorded.
performing 12-lead ECG for patients enrolled in the ¹⁷⁷Lu-PSMA-I&T after first dose.

(iii) On-Study Treatments

¹⁷⁷Lu-PSMA I&T: Patients randomized into the radioligand therapy arm of the study will receive ¹⁷⁷Lu-PSMA I&T infusions on a 6-week infusion cycle at a dose of 200 mCi (7.4 GBq) until radiographic progression is confirmed by the BICR or until the patient experiences toxicity requiring discontinuation of treatment or withdraws their consent to participate in the study. A maximum 4 cycles of ¹⁷⁷Lu-PSMA I&T infusions may be given to a patient in this study.

Abiraterone and Enzalutamide Standard of Care Arms: Patients randomized to standard of care and being treated with either abiraterone or enzalutamide will be treated daily with the standard of care dose regimen for these drugs according to their prescribing information. Patients in this treatment group will continue receiving the standard of care treatment until radiographic progression is confirmed by the BICR or until the patient experiences toxicity requiring discontinuation of treatment or withdraws their consent to participate in the study.

(iv) Ongoing Assessments

Ongoing assessments will be conducted for all patients enrolled in the study every 4 weeks for patients randomized to receive ¹⁷⁷Lu-PSMA I&T and patients receiving abiraterone or enzalutamide standard of care.

(v) Progression Assessments

Assessments will be conducted on all patients enrolled in the study starting 8 weeks+1 week from the initial treatment and continuing through Week 24, and thereafter every 12 weeks until radiographic evidence of disease progression is noted. The assessments consist of (i) obtaining CT chest, abdomen, and pelvis images plus bone scan and submit to ICL (Imaging Core Laboratory) for BICR to evaluate, and (ii) obtaining plasma samples for PSA and other clinical laboratory assessments.

(vi) End of Treatment Visit

The End of Treatment (EOT) visit will be conducted at one-month (±7 days) after the last infusion of ¹⁷⁷Lu-PSMA I&T. Patients receiving abiraterone acetate or enzalutamide can continue receiving daily treatment up until the EOT visit. The following assessments will be conducted: administering EORTC QLQ-C30, FACT-P, and BPI-SF questionnaires; recording any changes to concomitant medications; recording any adverse events noted from time of screening; performing abbreviated physical examination; performing and recording vital signs and ECOG performance status; and collecting blood for clinical laboratory assessments including PSA and other clinical laboratory assessments.

(vii) Crossover

Patients in the standard of care hormone therapy arm may crossover to receive ¹⁷⁷Lu-PSMA I&T based on the following criteria:
Must have documented radiographic progression by PCWG3-modified RECIST 1.1 by BICR while on the standard of care hormone therapy
Must not have started any other anti-cancer medications or therapy.
Participants with inadequate organ and bone marrow function as defined below will not be eligible for crossover:
   Absolute neutrophil count <1.5×109/L
   Platelet count <100×109/L.
   Hemoglobin <8 g/dL.
   AST/SGOT and/or ALT/SGPT >3.0×ULN.
   Total bilirubin >2×ULN unless patient has known Gilbert's syndrome and then may be 3×ULN.
   Creatinine clearance (CrCl)<50 mL/min based on the Cockcroft-Gault equation.
   Albumin ≥2.75 g/dL

If patients are not eligible for crossover, then they should instead complete the End-of-Study Visit and go into Long Term Follow-up.

(viii) Long-Term Follow-Up

Long-term patient follow-up will continue up to 5 years after the initial treatment in this study or until patient death or loss to follow-up. The following information will be collected:
Survival status, development of symptomatic disease progression, initiation of any new systemic anti-cancer therapies, progression on first subsequent therapy, and medical resource utilization every 4 months.
In addition, if patients discontinue study treatment prior to documented disease progression, obtain CT of the chest, abdomen, and pelvis plus bone scan and submit to BICR to evaluate disease progression every 12 weeks until documentation of radiographic disease progression.

### G. Statistical Considerations and Analytical Plan

(i) Sample Size

It is hypothesized that treatment of patients with ¹⁷⁷Lu-PSMA I&T will increase the radiographic Progression Free Survival (rPFS) from 6 months in the standard of care group to 10 months. Thus, a target Hazard Ratio (HR) under the alternative hypothesis of 0.60 is reasonable to expect for this Phase 3 study. Utilizing a 2:1 randomization, an estimated 237 progression events across both treatment arms will provide a 95% power to detect a statistically significant treatment effect using a two-sided log-rank test at an overall significance level of α=0.05. Given the anticipated accrual rates and follow times, these 237 progression events will occur in an estimated 269 patients.

It is also hypothesized that the treatment of patients with ¹⁷⁷LuPSMA I&T will increase the Overall Survival (OS) from 18 months in the standard of care group to 25 months. Thus, a target Hazard Ratio (HR) under the alternative hypothesis of 0.70 is reasonable to expect for this Phase 3 study.

Utilizing the larger HR (associated with OS) and a 2:1 randomization, an estimated 352 events across both treatment arms will provide a 95% power to detect a statistically significant treatment effect using a two-sided log-rank test at an overall significance level of α=0.0. Given the anticipated accrual rates and follow times, these 352 deaths will occur in an estimated 400 patients.

(ii) Planned Interim Analysis

The secondary outcome variable Overall Survival (OS) will have two interim analyses and a final analysis conducted. The first interim analyses will be conducted after approximately 25% (90) of the deaths have been observed and the second interim analysis will be conducted after approximately 75% (264) deaths have been observed. Then final analysis will be conducted after all planned 352 deaths are observed. It is anticipated the first interim analysis for OS will be conducted at the time when all 237 progression events will have been observed for the primary endpoint analysis.

The interim analyses and the final analysis for OS will be conducted using a two-sided log-rank test at a nominal significance level adjusted utilizing the standard O'Brien-Fleming spending function for a. The first interim analysis will have α=0.0006, the second interim analysis will have α=0.0151 and the final analysis of OS will be using a nominal significance level of α=0.047. These p-values satisfy the O'Brien-Fleming spending function for a cumulative α=0.05.

If the test of OS meets the nominal significance level at any of the interim analyses, the study will be deemed to be positive and patient accrual may stop, following review and approval by the Data Monitoring Committee (DMC).

(iii) Analysis Populations

A total of 400 patients are planned for this study.
Intention-to-treat (ITT) population: all randomized patients classified according to the treatment arms into which they were randomized, regardless of the actual treatment received.
Safety Population: all treated patients classified according to the actual treatment received, regardless of random assignment.
Cross over Population: All patients in the ITT population but where the analysis utilizes the information the patients were crossed over on treatment from the standard of care to ¹⁷⁷Lu-PSMA I&T. This will be utilized for the final OS analysis.

Other efficacy datasets (per protocol, evaluable, etc.) may be defined in the SAP (Statistical Analysis Plan), but the primary analyses of efficacy will be on the ITT population.

(iv) Analysis of Efficacy

The primary analysis of efficacy will use the ITT population. Unless stated otherwise, the analysis of secondary endpoints will be based on data collected during the randomized treatment period.

a. Radiographic Progression-Free Survival

rPFS is time from randomization to the first documented radiographic progressive disease or death due to any cause. The rPFS time of any living patient with no radiographic documented progression, or any patient starting other anti-cancer systemic therapies, will be censored at the date of last evaluable disease assessment on-study. The rPFS time of patients with no evaluable disease assessment on-study will be censored at randomization. The frequency of rPFS will be performed approximately every 8 weeks from time of first treatment through Week 24 and then every 12 weeks thereafter.

Distributions of rPFS times will be estimated using the Kaplan-Meier product-limit method. The median rPFS times with two-sided 95% CIs will be estimated for each treatment group. A log-rank test will be used as the primary analysis for comparison of treatment effects. A Cox proportional hazards model will be used to estimate the HR, and its two-sided 95% CI (Confidence interval).

b. Overall Survival

OS time is from randomization to death due to any cause. OS will be followed for 5 years post-enrollment in the study or until death or loss to follow-up. Patient data for the standard of care group will not be censored at the time of crossover.

Distributions of OS times will be estimated using the Kaplan-Meier product-limit method. The median OS times with two-sided 95% CIs will be estimated for each treatment group. A log-rank test will be used as the primary analysis for comparison of treatment effects. A Cox proportional hazards model will be used to estimate the hazard ratio, and its two-sided 95% CI.

Supplementary analyses may be performed if a sufficient proportion of patients crossover to ¹⁷⁷Lu-PSMA-I&T. The Rank Preserving Structural Failure Time (RPSFT) (Robins et al 1991) methodology will be utilized in this analysis.

c. Second Radiographic Progression Free Survival

rPFS2 is time from randomization to the second documented radiographic progressive disease or death due to any cause. rPFS2 is) defined as the time from randomization to the second radiographic progression (using PCWG3 criteria as assessed by blinded independent central review [BICR]) or death in participants who crossover from the standard of care hormone therapy arm to treatment with ¹⁷⁷Lu-PSMA I&T.

Distributions of rPFS2 times will be estimated using the Kaplan-Meier product-limit method. The median rPFS 2 times with two-sided 95% CIs will be estimated for each treatment group. A log-rank test will be used as the primary analysis for comparison of treatment effects. A Cox proportional hazards model will be used to estimate the HR, and its two-sided 95% CI.

d. Progression Free Survival

Progression is defined as the first occurrence of PCWG3 progression, clinical/symptomatic progression and/or pain progression, or death due to any cause. The time to progression will be assessed for all patients. Any patient with no documented progression, or any patient starting other anti-cancer systemic therapies, will be censored at the date of last evaluable disease assessment on-study.

Distributions of PFS will also be estimated using the Kaplan-Meier product-limit method. The median PFS times with two-sided 95% CIs will be estimated for each treatment group. A log-rank test will be used as the primary analysis for comparison of treatment effects. A Cox proportional hazards model will be used to estimate the HR (Hazard ratio) and its two-sided 95% CI.

Supplementary analyses may be performed if a sufficient proportion of patients crossover to ¹⁷⁷Lu-PSMA-I&T. The Rank Preserving Structural Failure Time methodology will be utilized in this analysis.

e. Second Progression-Free Survival

Progression-free survival 2, is the time from randomization to progression based on the following events, whichever occurs first: RECIST 1.1 progression, PCWG3 progression, clinical/symptomatic progression and/or pain progression, or death due to any cause as assessed by the Investigator.

Distributions of PFS2 will also be estimated using the Kaplan-Meier product-limit method. The median PFS2 times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients' crossover to ¹⁷⁷Lu-PSMA I&T. The Rank Preserving Structural Failure Time methodology will be utilized in this analysis.

f. PSA50 Response Rate

PSA50 is defined as the response rate of patients who achieve a reduction of ≥50% in PSA from the baseline PSA assessment. The PSA50 in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

g. Time to First Symptomatic Skeletal Event

The Time to First Symptomatic Skeletal (or SSE-free survival) event is defined as the occurrence of either bone-directed radiotherapy to relieve bony pain, new symptomatic pathologic fractures, spinal cord compression, or tumor-related orthopedic surgery. The time to SSE will be assessed for all patients. Any patient with no documented event, or any patient starting other anti-cancer systemic therapies, will be censored at the date of last evaluable disease assessment on-study.

Distributions of SSE will also be estimated using the Kaplan-Meier product-limit method. The median SSE times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

h. Time to Soft Tissue Progression

The Time to first radiographic soft tissue progression (STP) is defined as the occurrence of radiographic progression in soft tissue noted by PCWG3 modified RECIST 1.1. The time to STP will be assessed for all patients. Any patient with no documented event, or any patient starting other anti-cancer systemic therapies, will be censored at the date of last evaluable disease assessment on-study.

Distributions of STP will also be estimated using the Kaplan-Meier product-limit method. The median STP times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

i. Time to Chemotherapy

The Time to Chemotherapy (TTC) is defined as time from randomization to the initiation of chemotherapy or death, whichever comes first. The time to Chemotherapy will be assessed for all patients. Any patient with no documented event, or any patient starting other anti-cancer systemic therapies, will be censored at the date of last evaluable disease assessment on-study.

Distributions of TTC will also be estimated using the Kaplan-Meier product-limit method. The median TTC times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to 177Lu-PSMA I&T. The Rank Preserving Structural Failure Time methodology will be utilized in this analysis.

j. Quality of Life

The EORTC QLQ-C30 is a questionnaire of 30 QoL questions developed to assess the QoL of cancer patients. It has been translated and validated into 81 languages including English and Mandarin Chinese. Version 3.0 of the QLQ-C30 will be used in this Phase 3 study. The QoL questionnaire has been used as an efficacy endpoint in over 3,000 Phase 3 cancer clinical trials.

The EORTC questionnaires will be given to the patients at baseline, Day 8 of each cycle, and at the EOT. There are numerous statistical techniques that have been presented in the literature to analyze QoL data. The statistical methods to be used will be included in the SAP.

k. Objective Response Rate

Objective response rate (ORR) to protocol treatment will be evaluated by RECIST version 1.1, and the best overall response will be classified as CR, PR, stable disease, progressive disease (PD), and not evaluable (NE). ORR is defined as the proportion of patients who achieve either a CR or a PR. CT, and bone scans as applicable, at baseline and during study will be reviewed by independent radiologists at a central imaging laboratory to determine objective response, date of response, and progression.

The ORR in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

A further analysis of the ORR may be performed utilizing the Cross over population.

I. Disease Control Rate

Disease Control Rate (DCR) to protocol treatment will be evaluated by RECIST version 1.1, and the best overall response will be classified as CR (Complete response), PR (Partial response), stable disease, progressive disease (PD), and not evaluable (NE). DCR is defined as the proportion of patients who achieve disease control.

The DCR in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

A further analysis of the DRR may be performed utilizing the Cross over population.

m. Duration of Response

In patients who achieve the best objective response of CR or PR, DoR is time from the first observation of CR or PR (whichever status occurs first) to the first documented progressive disease. DoR of any patient with no documented progression, or any patient starting other anti-cancer systemic therapies, will be censored at the date of last evaluable disease assessment on-study.

Distributions of DoR will also be estimated using the Kaplan-Meier product-limit method. The median DoR times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients' crossover to ¹⁷⁷Lu-PSMA I&T. The Rank Preserving Structural Failure Time methodology will be utilized in this analysis.

n. Time to PSA Progression

The time to PSA progression as assessed by a ≥25% increase in PSA from the post-treatment nadir or, if no nadir, the baseline PSA assessment. The time to PSA progression of any patient with no documented progression, or any patient starting other anti-cancer systemic therapies, will be censored at the date of last evaluable disease assessment on-study.

Distributions of time to PSA progression will also be estimated using the Kaplan-Meier product-limit method. The median time to PSA progression with two-sided 95% CIs will be estimated for each treatment group. A log-rank test will be used as the primary analysis for comparison of treatment effects. A Cox proportional hazards model will be used to estimate the HR, and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

(vi) Clinical Laboratory Evaluations

Clinical laboratory results may be collected pretreatment through 28 days after the last dose of any study therapy. All clinically significant laboratory abnormalities namely tests that result in treatment modification and/or require intervention, may be recorded as AEs.

### H. End of Trial

Patients may remain on study treatment until BICR-confirmed disease progression, development of unacceptable toxicity, or withdrawal of consent. Patients discontinuing study treatment may enter the Long-term Follow-up Phase and remain on study until death, loss of follow-up, or withdrawal of consent, whichever comes first.

With an estimated accrual duration of 12-24 months, it is assumed that human patients may be followed for a minimum of approximately 34 weeks beyond Last Patient In (LPI) for the primary endpoint of radiographic progression free survival, to 5 years beyond LPI for the secondary endpoint of OS. This corresponds to total projected study duration of approximately 6-7 years.

### Example 6: Pharmacokinetics and Radiation Dosimetry Sub-Study

A. Objectives

The objective of this study is to evaluate the plasma pharmacokinetic and radiation dosimetry of ¹⁷Lu-PSMA &T radioligand therapy in a subset of 30 patients.

This sub-study is designed to evaluate the plasma pharmacokinetic profile and radiation dosimetry of ¹⁷⁷Lu-PSMA I&T radioligand therapy over 4 treatment cycles.

B. Study Design and Patient Population

Representative batch certification of the batches of ¹⁷Lu-PSMA I&T used in the study is provided in Table 18.

TABLE 18
Representative certificate of analysis of ¹⁷⁷Lu-PSMA I&T.

| | | |
|---|---|---|
| Characteristics | Specification | Result |

Release Tests

| Appearance | Clear, colorless or slightly yellow liquid, | | Pass |
|---|---|---|---|
| | no visible particles | | |
| pH | 4.0-5.0 ± 0.5 | | 4.5 ± 0.5 |
| Ascorbic acid concentration | 15-35 mg/ml ± 5 mg/ml | | Pass |
| Radionuclidic identification | Gamma ray energy | | 113 keV ± 10 keV |
| | HPGe | Nal | 209 keV ± 12 keV |
| | 113 keV ± 2 keV | 113 keV ± 10 keV | |
| | 208 keV ± 4 keV | 208 keV ± 12 keV | |

| | | No other significant peaks with gamma | Pass |
|---|---|---|---|
| | | energy >100 keV are detected | |
| Radiochemical Identity | | The radiometric RT of the test sample | Pass |
| | | corresponds to UV RT of Reference | |
| | | Standard ±5.0%. | |
| Radiochemical | by HPLC | ≥95.0% as ¹⁷⁷Lu-PSMA | 98.2% ± 1.8% mg/ml |
| purity | by TLC | ¹⁷⁷Lu-colloid ≤5.0% | 0% |

| | | |
|---|---|---|
| Chemical purity | Lu-PSMA-I&T concentration | 1.4 μg/ml ± 0.8 μg/ml |
| | ≤6.0 μg/ml | (i.e., ≤3.0 μg/ml) |
| Ethanol content (w/w) | 2.5%-4.5% ± 1.5% | 3.7% ± 0.7% |
| ¹⁷⁷LU-PSMA-I&T | Reported | 168 GBq/μmol ± |
| Specific activity | | 50 GBq/μmol |
| Endotoxins | <175 EU/20 mL vial | Pass |

Post Release Tests

| | | |
|---|---|---|
| Sterility | Sterile | Pass |
| Radionuclidic purity | ≥99.9% of the total activity of 177Lu | Pass |
| | at the end of the operating life | |

The plasma pharmacokinetic profile and radiation absorbed dose of ¹⁷⁷Lu-PSMA I&T radioligand therapy will be assessed in a subset of 30 patients enrolled that will be participating in the study of Example 5. The subset of 30 patients will undergo the same screening procedures as the main protocol of Example 5 in order to be randomized into the study, following the same Inclusion/Exclusion criteria of Example 5, with the following additional enrollment criteria:

Inclusion Criteria
Enrollment in the main study (Example 5).
Separate informed consent for participation in the sub-study.
Willing to undergo planar and/or SPECT/CT imaging at 4 hours, 24 hours, 48 hours, and 6-8 days after each ¹⁷⁷Lu-PSMA I&T treatment cycle (Main study Cycles 1-6).

Exclusion Criteria
Unable to undergo SPECT/CT imaging as required in the sub-study protocol.

C. Methods

The plasma pharmacokinetic profile will be determined in the subset of 30 patients enrolled in this study by acquiring plasma samples prior to and at approximately 1, 4, 24, and 48 hours and 6-8 days after the completion of each ¹⁷⁷Lu-PSMA I&T infusion. The time of completion of ¹⁷⁷Lu-PSMA I&T infusion and the actual times of sample collection will be recorded. Radioactivity will be assayed in plasma samples using a calibrated well counter, and the percent of the injected dose will be calculated after correcting the radioactive decay half-life of 6.647 days for ¹⁷⁷Lu.

Planar whole body scintigraphic images will be acquired at approximately 4, 24, and 48 hours and at 6-8 days following completion of each ¹⁷⁷Lu-PSMA I&T infusion. SPECT/CT images of the upper abdomen, kidneys, and salivary glands will be acquired at approximately 24 hours and 6-8 days following each ¹⁷⁷Lu-PSMA I&T infusion. The actual start time and date for all scintigraphic imaging will be recorded on the CRF.

Imaging data will be submitted to a core imaging lab for processing. For tumor dosimetry calculations, regions of interests (ROIs) showing negligible overlap with high physiologic uptake or other positive lesions on scintigraphy will be selected. Background ROIs will be drawn from outside of the body. At a minimum, ROIs will be selected for the whole body, kidneys, liver, parotid glands, submandibular glands, and lacrimal glands, as well as tumor ROIs and other organs demonstrating significant uptake of the ¹⁷⁷Lu-PSMA I&T.

Following the administration/infusion of a dose of a ¹⁷⁷Lu-PSMA I&T solution, the human patient(s) may undergo single photon emission computed tomography (SPECT)/computed tomography (CT) imaging, e.g., at four timepoints (4 h, 24 h, 48 h 168 h). Image data may be analyzed to compute time integrated activity coefficients (TIAC) in each organ of interest and/or with appreciable and meaningful 177Lu-PSMA I&T activity above background which included kidneys, bladder, liver, lumbar vertebrae L2-L4, lacrimal glands, salivary glands, intestine, whole body, and combinations thereof. To compute subject-specific organ doses, organ-level TIAC data may be entered into Organ Level Internal Dose Assessment, OLINDA 2.2.3 and resulting organ doses for each target organ (e.g., kidney) and whole body effective dose may be collated and averaged.

D. Schedule of Activities Specific to the Pharmacokinetic and Radiation Dosimetry Sub-Study

(i) Screening
Verify inclusion/exclusion criteria are met
Obtain informed consent for participation in sub-study

(ii)¹⁷⁷Lu-PSMA I&T Infusion Cycles 1-4
Obtain plasma samples prior to and at approximately 1, 4, 24, and 48 hours and 6-8 days after the completion of each ¹⁷⁷Lu-PSMA I&T infusion as shown in Table 19.
Obtain planar whole body scintigraphic images at approximately 4, 24, and 48 hours and 6-8 days following completion of each ¹⁷⁷Lu-PSMA I&T infusion.
Obtain SPECT/CT images at approximately 24 hours and 6-8 days following completion of each ¹⁷⁷Lu-PSMA I&T infusion.

TABLE 19
Schedule of time points to take samples.

Activities

| Specific to Sub- | | Time After Each ¹⁷⁷Lu-PSMA I&T Infusion (Cycles 1-4) | | | | | |
|---|---|---|---|---|---|---|---|
| study | Screening | Prior to | 1 hr | 4 hr | 24 hr | 48 hr | 6-8 days |
| Informed consent | X | | | | | | |
| Plasma PK | | X | X | X | X | X | X |
| sampling | | | | | | | |
| Whole Body | | | | X | X | X | X |
| Planar Imaging | | | | | | | |
| SPECT/CT Imaging | | | | | X | | X |

E. Statistical Methods

(i) Pharmacokinetic Parameters

The following PK parameters derived from whole blood radiation counts of ¹⁷⁷Lu-PSMA-I&T infusion will determined as appropriate:
AUC_{0-24; N-last}: area under the whole blood radiation counts time curve (0 to 24 hours, 0 to last) calculated by use of the trapezoidal rule
AUC_{0-∞}: area under the whole blood radiation counts time curve from AUC₀₋₂₄ or AUC₀₋ₗₐₛₜ (calculated by use of the trapezoidal rule)+area under the curve estimated by Cpₙ/kₑₗ, where:
   Cpₙ=last observed whole blood radiation count at time n
   kₑₗ=elimination rate constant, calculated from the log-linear terminal portion of the whole blood radiation counts time curve
Cₘₐₓ: observed maximum whole blood radiation count
Tₘₐₓ: time to Cₘₐₓ
kₑₗ: elimination rate constant, calculated from the log-linear terminal portion of the whole blood radiation counts time curve
t_{1/2}: apparent elimination half-life, calculated as ln2/kₑₗ
CL: total body clearance for extravascular administration at steady-state, calculated as Dose/AUC₀₋₂₄
V_{d}: volume of distribution

(ii)¹⁷⁷Lu-PSMA I&T infusion Radiation Counts
C(t): The radiation counts measured at time=t were tabulated for time and each patient. Whole blood radiation counts below the limit of quantitation were treated as zero (0).

(iii) Area Under the Curve

The following characteristics will be calculated for all complete profiles, if not stated otherwise:
AUC_{0-24, 0-last}: The area under the whole blood radiation counts C(t) from administration will be calculated by use of the trapezoidal rule.

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention.

### Example 7: Comparative Study for Extension of Product Expiration from 48 Hours to 74 Hours for ¹⁷⁷Lu-PSMA I&T

The objective of this study was to extend the expiry period of ¹⁷⁷Lu-PSMA I&T Injection from 48 hours to 72 hours. The 72-hour expiry was achieved by modifying the drug product composition, by reducing the strength of the ¹⁷⁷Lu-PSMA I&T from 1 GBq/mL to 0.5 GBq/mL and maintaining a more restrictive pH of the formulated drug product in the range of 4 to 5. The total patient dose was unchanged, which remains at 7.4 GBq ¹⁷⁷Lu-PSMA I&T. In order to deliver the desired radioactive dose, the volume of drug was increased from 8-10 mL to 15-20 mL. Thus, the primary container closure system was changed from a 10 mL to a 20 mL vial, while maintaining the same glass quality, the same elastomeric closure, and the same aluminum crimp seal (Table 19).

In this study, the stability of “Drug Product B” was studied and resulted in extended expiration period of ¹⁷⁷Lu-PSMA I&T Injection of “Drug Product A”.

A. Description and Composition of the Drug product

TABLE 20
Description of Drug Product

| Drug Product A | Drug Product B |
|---|---|
| The drug product was packaged | The drug product was packaged |
| in a 10 mL sterile glass vial | in a 20 mL sterile glass vial |
| with a Fluorotec coated 4432.50 | with a Fluorotec coated 4432.50 |
| chlorobutyl pharmaceutical-grade | chlorobutyl pharmaceutical-grade |
| rubber stopper secured in place | rubber stopper secured in place |
| with an aluminum crimp seal. | with aluminum crimp seal. |

Table 20 provides a listing of the qualitative and quantitative composition of ¹⁷⁷Lu-PSMA I&T Injection, which had undergone a composition change to increase the shelf-life, which was achieved by reducing the strength (radioactive concentration) from 1 GBq/mL to 0.5 to 0.6 GBq/mL, using a more stringent pH, and increasing the fill volume to −20 mL/vial. The number of vials produced per batch was adjusted in order to produce the number of doses required for therapeutic administration. The drug product qualitative and quantitative composition of ⁷⁷Lu-PSMA I&T are provided in Table 21.

TABLE 21
Drug Product Qualitative and Quantitative Composition of ¹⁷⁷Lu-PSMA I&T

| | Drug Product A | | Drug Product B | | |
|---|---|---|---|---|---|
| | Quantity | In 1 dose 7.4 | Quantity | In 1 dose 7.4 | |
| Component | per mL | GBq (10 mL) | per mL | GBq (20 mL) | Function |
| ¹⁷⁷Lu-PSMA I&T | 1 | | 0.5 | | Radioactive |
| (GBq) | | | | | pharmaceutical |
| | | | | | ingredient |
| Typical volume for | | 8-9 (Max | | 15 (15-20 | |
| 1 dose (mL) | | 10 mL) | | 10 mL)** | |
| ¹⁷⁷Lu-PSMA I&T | <12 | <120 | <6 | <120 | |
| and PSMA-I&T (μg) | | | | | |
| Ascorbic acid (mg) | 42.5 | Formulation: | 31 | Formulation: | Antioxident |
| | (20-70) | 340-425 | (15-35) | 340-425 | |
| | | Specification: | | Specification: | |
| | | 200-700 | | 200-700* | |
| Disodium EDTA | 21 | 168-210 | 15.5 | <310 | Metal ion chelator, |
| (μg) | | | | | present as |
| | | | | | excipient in |
| | | | | | formulated |
| | | | | | ascorbic acid |
| | | | | | injection |
| Sodium | A | QS | A | | Present as |
| Bicarbonate | sufficient | | sufficient | | excipient in |
| | quantity | | quantity | | formulated |
| | | | | | ascorbic acid |
| | | | | | injection |
| NaOH | A | QS | A | | Present as |
| | sufficient | | sufficient | | excipient in |
| | quantity | | quantity | | formulated |
| | | | | | ascorbic acid |
| | | | | | injection |
| Hydrochloric | N/A | N/A | A | N/A | pH adjustment |
| acid | | | sufficient | | |
| (Max addition of | | | quantity | | |
| 30% HC1 is | | | | | |
| 468 μ1/100 m1 final | | | | | |
| formulation, i.e. | | | | | |
| should not have | | | | | |
| any relevant impact | | | | | |
| on chloride ion | | | | | |
| quantities) | | | | | |
| Ethanol (μL) | 50-80 (5- | Formulation: | | Formulation: | Vehicle/Stabilizing |
| | 8% v/v) | 600-750 | | 563-750 | agent (radiolysis) |
| | | Specification: | | Specification: | |
| | | 500-800 | | 375-900 (2.5 | |
| | | (5-8% v/v) | | to 4.5% v/v) | |
| Water for Injection | Sufficient | QS | Sufficient | QS | Vehicle |
| | to make 1 | | to make 1 | | |
| | mL | | mL | | |
| pH*** | | 5-8 | | 4-5 | |

*North American medical authorities have set a maximum adult dose of 2,000 mg per day, so 700 mg is acceptable.
**The recommended dose of ¹⁷⁷Lu-PSMA I&T is 7.4 GBq (200 mCi)/administration. Depending on the actual radioactive strength of each batch, the volume dispensed into the 20 mL vial may vary between ~15-20 mL.
***The updated pH range is within the range is within the range of other FDA-approved USP grade injectable solutions (ex: 5% dextrose injection solution).

The changes associated with the expiration extension did not change the identity, quality, or purity of any of the components of the drug substance or drug product. It did change the strength of the API in the final drug product, established a narrower control range for pH, and modified the concentration of the two main excipients, ascorbic acid and ethanol. These changes were made in order to extend the expiry period of the drug product.

With these changes associated with the expiration extension, the batch size was scaled to provide the number of therapy doses, plus the overage needed to provide quality control test samples for both microbiological and chemistry tests and to provide a reserve sample for each batch. In this context, all components and excipients were scaled to the measured amount of radioactive Lu-177 precursor labeling solution.

B. Manufacturing Process

This expiration extension study did not involve any substantive changes to the manufacturing process, equipment, or reagents used. The reformulated product was formulated to a lower radioactive concentration (strength), more stringent pH limits applied, and the same excipients used, but in different strengths from the original formulation. As the manufacturing chemistry of the drug substance remained unchanged, the changes only applied to the formulation of the final drug product solution.

The pH was the only critical step that was updated. The pH of the final drug product solution was controlled to the narrower range of 4.0 to 5.0 from 5.0 to 8.0. This was accomplished by the controlled addition of hydrochloric acid.

The stability of the product was evaluated over the assigned shelf life at storage temperatures ranging from 2° C.-40° C. In addition, microbial bioburden studies were performed on three other batches of ¹⁷⁷Lu-PSMA I&T injection. All batches met pre-determined acceptance criteria through the extended 72 hour expiration time point. The product composition was changed, to decrease the target strength from −1 GBq/mL to between 0.5 and 0.6 GBq per mL. The pH range was also controlled to the narrower range of 4.0 to 5.0 from 5.0 to 8.0, and due to the lower strength formulation, the dispensed volume was increased from 10 to 20 mL/vial. In addition, the strength of ascorbic acid and ethanol was decreased. This lower strength product was subjected to process validation studies that confirmed that the 72-hour expiration date had been achieved.

A media fill study was successfully executed on three test batches to support aseptic fill operations corresponding to changes in drug product volume (increase) and vial size.

There are no changes to the excipients utilized in the drug product formulation, which remain as (Ethanol, Ascorbic Acid, and Water for Injection). With drug product composition change, it did not result in the creation of any new impurities or an increase in existing impurities. Only the concentrations/volume of existing excipients were adjusted to achieve a larger volume.

Table 22 below lists Drug Product A and Drug Product B tests and acceptance criteria.

TABLE 22
Tests and Acceptance Criteria for Drug Product A
and Drug Product B of ¹⁷⁷Lu-PSMA-I&T injection.

| | Acceptance Requirement | |
|---|---|---|
| Test | Drug Product A* | Drug Product B* |
| Appearance | Clear and colorless to | Clear and colorless to |
| | yellow solution, no | yellow solution, no |
| | visible particles | visible particles |
| pH | 5.0-8.0 | 4.0-5.0 |
| Ascorbic Acid | 20-70 mg/mL | 15-35 mg/mL |
| Radionuclidic identification | Energy from ¹⁷⁷Lu main | Energy from ¹⁷⁷Lu main |
| | peaks: 113 ± 10 keV and | peaks: 113 ± 10 keV and |
| | 208 ± 12 keV No other | 208 ± 12 keV No other |
| | significant peaks with | significant peaks with |
| | gamma energy >100 keV | gamma energy >100 keV |
| | are detected | are detected |
| Chemical purity | Lu-PSMA I&T - | Lu-PSMA I&T - |
| | concentration ≤12.0 | concentration ≤6.0 |
| | μg/mL | μg/mL |
| Radiochemical Identity | The radiometric RT of the | The radiometric RT of the |
| | test sample = UV RT of | test sample = UV RT of |
| | Reference Standard ±5%. | Reference Standard ±5%. |
| Radiochemical purity by | ≥95.0% | ≥95.0% |
| HPLC method | | |
| Radiochemical purity by | ¹⁷⁷Lu-colloid ≤5.0% | ¹⁷⁷Lu-colloid ≤5.0% |
| TLC method | | |
| Radionuclidic purity | ≥99.9% of the total | ≥99.9% of the total |
| | activity of ¹⁷⁷Lu at the end | activity of ¹⁷⁷Lu at the end |
| | of the operating life | of the operating life |
| Endotoxin | <175 EU/10 mL vial* | <175 EU/10 mL vial* |
| | | <8.75 EU/mL |
| Ethanol | 5.0% to 8.0% (v/v) | 2.5% to 4.5% (v/v) |
| Sterile filter integrity | ≥3.45 bar (Millex GV) | ≥3.32 bar (Pall, Mini |
| | | Kleepak) ** |
| Sterility*** | Sterile | Sterile |

*Dose = maximum volume to be administered per dose is defined as 10 mL (200 mCi) for original formulation and 20 mL for the new formulation (200 mCi)
**There was no change to the sterilizing filter.
***The sterility test samples are initiated post-release.

C. Analytical Procedures

Due to the decrease in strength (radioactive concentration) of the product (per mL), the sample injection volume was increased from 50 μL to 100 μL for some of the drug product tests. Due to this change, the following method verification studies were performed:
Determination of Colloidal Impurities in Lu-177-PSMA Injection by TLC
Bioburden—Microbial Growth
Endotoxin Testing
Sterility Testing
Determination of Ethanol by Gas Chromatography

The decrease in strength (radioactive concentration) resulted in a slower rate of radiolysis, such that the radiochemical purity at 72 hours post-end-of-synthesis remained above 95%. In effect, this did not result in the creation of any new impurities or an increase in existing impurities

D. Container Closure System

The vial in the primary container closure system was changed from a 10 mL glass pharmaceutical-grade injection vial to a 20 mL vial of the same pharmaceutical grade and neck finish. It was sealed with the current fluoro coated bromobutyl elastomeric closure secured in position with an open-top aluminum crimp. Neither the elastomeric closure nor crimp was changed (Table 23).

TABLE 23
Container Closure Comparison of
Drug Product A and Drug Product B

| Component | Drug Product A | Drug Product B |
|---|---|---|
| Vial, 10 mL | Schott (Glass Vial) | NA |
| | Adelphi (Sterile, | |
| | air-filled) | |
| Vial, 20 mL | NA | Curium/US code 5935 |
| | | 2703 Wagner Place |
| | | St. Louis, Missouri |
| | | 63042, USA |
| Elastomeric Closure | West Pharmaceutical | West Pharmaceutical |
| | Services 1028 Innovation | Services 1028 Innovation |
| | Way Kinston, NC 28504 | Way Kinston, NC 28504 |
| Aluminum Crimp | GI Pharma | GI Pharma |

E. Stability

The radiochemical purity and chemical properties of the 0.5 GBq/mL ¹⁷⁷Lu PSMA-I&T injection solution (inclusive of pH, impurities, and visual properties) were tested on three process validation and four separate stability batches over a time span of 72 hours from the end-of-synthesis time. All batches utilized the ¹⁷⁷Lu precursor labeling solution. Stability samples from all batches of ¹⁷⁷Lu-PSMA-I&T injection drug product were stored at room temperature, with selected samples also stored inverted and some samples stored at elevated temperatures. Initial tests were generally completed within 7 hours post end of synthesis, and stability-indicating tests were repeated at 24, 48, and −72 hours post EOS. No deviations from specification acceptance criteria were observed for samples tested up to and including 72 hours for samples formulated at a strength of −0.5 GBq/mL. Based on these supportive stability data, an expiration time of 72 hours post-end-of-synthesis was assigned. The changes in the Shelf life of the drug product is provided in Table 24.

TABLE 24
Shelf Life comparison of Drug Product A and Drug Product B

| Attribute | Drug Product A | Drug Product B |
|---|---|---|
| Shelf-Life (Expiry) | 48 Hrs Post End of | 72 Hrs Post End of |
| | Synthesis | Synthesis |

F. Conclusion

The 72-hour expiry was achieved by modifying the drug product composition, by reducing the strength of the ¹⁷⁷Lu PSMA I&T from 1 GBq/mL to 0.5 GBq/mL and maintaining a more restrictive pH of the formulated drug product in the range of 4 to 5.

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention.

Numerous examples are provided herein to enhance the understanding of the present disclosure. A specific set of statements are provided as follows.
Statement 1: A radiopharmaceutical composition comprising ¹⁷⁷Lu-PSMA I&T, wherein the composition is formulated as a solution for injection and the solution is suitable for administration more than 72 hours after formulation.
Statement 2: The radiopharmaceutical composition of statement 1, wherein the solution is suitable for administration up to 3 days after formulation.
Statement 3: The radiopharmaceutical composition of statement 1, wherein the solution is suitable for administration up to 4 days after formulation.
Statement 4: The radiopharmaceutical composition of statement 1, further comprising an antioxidant.
Statement 5: The radiopharmaceutical composition of statement 4, wherein the antioxidant is ascorbic acid.
Statement 6: The radiopharmaceutical composition of statement 5, wherein the solution comprises 21 mg/ml to 31 mg/ml ascorbic acid.
Statement 7: The radiopharmaceutical composition of statement 5, wherein the solution comprises 42.5 mg/ml ascorbic acid.
Statement 8: The radiopharmaceutical composition of statement 1, wherein the solution further comprises hydrochloric acid.
Statement 9: The radiopharmaceutical composition of statement 1, wherein the solution comprises 1.7 mg/ml to 34 mg/ml hydrochloric acid.
Statement 10: The radiopharmaceutical composition of statement 1, wherein the solution has a pH of 4.5 or below.
Statement 11: The radiopharmaceutical composition of statement 1, wherein the solution has a pH of 5 or below.
Statement 12: The radiopharmaceutical composition of statement 1, wherein the solution has a radioactivity of less than 635 MBq/ml.
Statement 13: The radiopharmaceutical composition of statement 12, wherein the solution has a radioactivity of 579 MBq/ml to 626 MBq/ml.
Statement 14: The radiopharmaceutical composition of statement 1, wherein the solution has a radiochemical purity of more than 95% at 46 to 48 hours after formulation.
Statement 15: The radiopharmaceutical composition of statement 14, wherein the solution has a radiochemical purity of more than 96% at 46 to 48 hours after formulation.
Statement 16: The radiopharmaceutical composition of statement 14, wherein the solution has a radiochemical purity of more than 97% at 46 to 48 hours after formulation.
Statement 17: The radiopharmaceutical composition of statement 1, wherein the solution has a radiochemical purity of more than 95% at 69 to 71 hours after formulation.
Statement 18: The radiopharmaceutical composition of statement 17, wherein the solution has a radiochemical purity of more than 96% at 69 to 71 hours after formulation.
Statement 19: The radiopharmaceutical composition of statement 17, wherein the solution has a radiochemical purity of more than 97% at 69 to 71 hours after formulation.
Statement 20: The radiopharmaceutical composition of statement 1, wherein the solution has a radiochemical purity of more than 95% at 90 to 93 hours after formulation.
Statement 21: The radiopharmaceutical composition of statement 20, wherein the solution has a radiochemical purity of more than 96% at 90 to 93 hours after formulation.
Statement 22: The radiopharmaceutical composition of statement 20, wherein the solution has a radiochemical purity of more than 97% at 90 to 93 hours after formulation.
Statement 23: The radiopharmaceutical composition of statement 1, wherein the solution comprises less than 6 μg/ml Lu-PSMA I&T.
Statement 24: The radiopharmaceutical composition of statement 1, further comprising a metal ion chelator.
Statement 25: The radiopharmaceutical composition of statement 24, wherein the metal ion chelator is disodium EDTA.
Statement 26: The radiopharmaceutical composition of statement 25, wherein the solution comprises about 15.5 μg/ml disodium EDTA.
Statement 27: The radiopharmaceutical composition of statement 1, further comprising a stabilizing agent.
Statement 28: The radiopharmaceutical composition of statement 27, wherein the stabilizing agent is ethanol. The radiopharmaceutical composition of statement 27, wherein the stabilizing agent is not ethanol.
Statement 29: The radiopharmaceutical composition of statement 28, wherein the solution comprises about 37.5 μl/ml ethanol.
Statement 30: The radiopharmaceutical composition of statement 1, further comprising 31 mg/ml ascorbic acid and an amount of hydrochloric acid adjusted such that the solution has a pH of 4.5.
Statement 31: The radiopharmaceutical composition of statement 30, further comprising 15.5 μg/ml disodium EDTA and 37.5 μL/ml ethanol.
Statement 32: The radiopharmaceutical composition of statements 30 or 31, further comprising sodium bicarbonate and NaOH in sufficient amounts to control pH to 4.5.
Statement 33: The radiopharmaceutical composition of statement 30, wherein the solution has a radioactivity of 588.5 MBq/ml.
Statement 34: The radiopharmaceutical composition of statement 30, wherein the solution has a radiochemical purity of at least 98% at 44 hours after formulation, at least 97% at 69 hours after formulation, and/or at least 97% at 93 hours after formulation.
Statement 35: A radiopharmaceutical composition comprising ¹⁷⁷Lu-PSMA I&T and ascorbic acid at a pH of 3.5 to 4.5 in solution, wherein the composition is suitable for administration to a human patient in need thereof at least 90 hours after formulation, and wherein the composition has a radiochemical purity of 95% or greater at administration.
Statement 36: The radiopharmaceutical composition of statement 35, wherein the composition comprises <6 μg of Lu-PSMA &T per mL of solution.
Statement 37: The radiopharmaceutical composition of statement 35, wherein the composition comprises about 13 μg to about 18 μg disodium EDTA per mL of solution.
Statement 38: The radiopharmaceutical composition of statement 35, wherein the composition comprises about 35 μL to about 40 μL ethanol per mL of solution.
Statement 39: The radiopharmaceutical composition of statement 35, wherein the composition comprises a radioactivity of about 0.5 GBq or about 13.5 mCi per mL of solution.
Statement 40: The radiopharmaceutical composition of statement 35, wherein the composition comprises about 31 mg/ml ascorbic acid.
Statement 41: The radiopharmaceutical composition of statement 35, wherein the composition comprises about 21 mg/ml to about 31 mg/ml ascorbic acid.
Statement 42: The radiopharmaceutical composition of statement 35, wherein the composition comprises about 31 mg/ml to about 42.5 mg/ml ascorbic acid.
Statement 43: A radiopharmaceutical composition comprising ¹⁷⁷Lu, about 463 μg/mL PSMA &T precursor, about 4 ml 0.4 M sodium acetate, about 1.6 mL 0.05 M hydrochloric acid, about 150 μl 20% L-ascorbic acid, and a specific activity ≤61 GBq in a total of 6 to 8 mL of solution.
Statement 44: The radiopharmaceutical composition of statement 43, wherein the ascorbic acid has a pH of 4.5.
Statement 45: The radiopharmaceutical composition of statement 43, further comprising 1.5 ml ethanol-water in a 1:1 (v/v) ratio.
Statement 46: A radiopharmaceutical composition comprising ¹⁷⁷Lu-PSMA &T and ascorbic acid at a pH of 4.5 in solution, wherein the composition is suitable for administration to a human patient in need thereof at least 93 hours after formulation, and wherein the composition has a radiochemical purity of 97.0% or greater at administration.
Statement 47: A radiopharmaceutical composition comprising ¹⁷⁷Lu-PSMA I&T and ascorbic acid at a pH of 4.5 in solution, wherein the composition is suitable for administration to a human patient in need thereof at least 92 hours after formulation, and wherein the composition has a radiochemical purity of 96.0% or greater at administration.
Statement 48: A radiopharmaceutical composition comprising ¹⁷⁷Lu-PSMA I&T, wherein the composition is formulated as a solution suitable for injection and the solution has a pH of 4.5.
Statement 49: A radiopharmaceutical composition comprising ¹⁷⁷Lu-PSMA I&T, wherein the composition is formulated as a solution suitable for injection and the solution has a radiochemical purity of more than 96% more than 48 hours after formulation.
Statement 50: A radiopharmaceutical composition comprising ¹⁷⁷Lu-PSMA I&T, wherein the composition is formulated as a solution suitable for injection and the solution has a radiochemical purity of more than 96% more than 71 hours after formulation.
Statement 51: A radiopharmaceutical composition of any of the preceding statements, wherein upon administration of the composition to a subject, the subject maintains low levels of hematotoxic and nephrotoxic toxicity, and wherein the prostate-specific antigen decline is more than about 50%.
Statement 52: A method of preparing ¹⁷⁷Lu-PSMA I&T comprising heating the composition of statement 43 up to 95° C. for 15 minutes.
Statement 53: The method of statement 52, further comprising adjusting the solution pH to 3.5 to 4.5.
Statement 54: A method of administering a radiopharmaceutical solution, the method comprising injecting the radiopharmaceutical solution more than 48 hours after formulation, wherein the radiopharmaceutical solution comprises ¹⁷⁷Lu-PSMA I&T.
Statement 55: A method of administering a radiopharmaceutical solution, the method comprising injecting the radiopharmaceutical solution comprising ¹⁷⁷Lu-PSMA I&T, wherein the solution has a pH of 4.5.
Statement 56: A method of administering a radiopharmaceutical solution, the method comprising injecting the radiopharmaceutical solution comprising ¹⁷⁷Lu-PSMA I&T, wherein the solution has a radiochemical purity of more than 96% more than 48 hours after formulation.
Statement 57: A method of administering a radiopharmaceutical solution, the method comprising injecting the radiopharmaceutical solution comprising ¹⁷⁷Lu-PSMA I&T, wherein the solution has a radiochemical purity of more than 96% more than 71 hours after formulation.
Statement 58: A radiopharmaceutical composition comprising ¹⁷⁷Lu-PSMA I&T, about 31 mg/ml ascorbic acid, about 13 μg/ml to about 18 μg/ml disodium EDTA, and about 35 μl/ml to about 40 μl/ml ethanol in a solution having a pH of 3.5 to 4.5, wherein the solution is suitable for administration more than 48 hours after formulation and has a radiochemical purity of more than 96% when administered.
Statement 59: A method of administering a radiopharmaceutical composition, the method comprising injecting the radiopharmaceutical composition into a patient in need thereof more than 48 hours after formulation, the radiopharmaceutical composition comprising ¹⁷⁷Lu-PSMA I&T and ascorbic acid in a solution having a pH of 3.5 to 4.5, wherein the solution has a radiochemical purity of more than 96% when administered.
Statement 60: The method of statement 59, wherein the pH is 3.5 to 4.2.
Statement 61: The statement of claim 59, wherein the composition comprises <6 μg of Lu-PSMA I&T per mL of solution.
Statement 62: The method of statement 59, wherein the composition comprises about 13 μg to about 18 μg disodium EDTA per mL of solution.
Statement 63: The method of statement 59, wherein the composition comprises about 35 μL to about 40 μL ethanol per mL of solution.
Statement 64: The method of statement 59, wherein the composition comprises a radioactivity of about 0.5 GBq or about 13.5 mCi per mL of solution.
Statement 65: The method of statement 59, wherein the composition comprises about 31 mg/ml ascorbic acid.
Statement 66: The method of statement 59, wherein the solution has a radiochemical purity of at least 98% at 44 hours after formulation, at least 97% at 69 hours after formulation, and/or at least 97% at 93 hours after formulation.
Statement 67: The method of statement 59, wherein upon administration of the composition to a patient, the patient maintains low levels of hematotoxic and nephrotoxic toxicity, and wherein the prostate-specific antigen decline is more than about 50%.
Statement 68: A method of treating a patient with mCRP in need thereof, the method comprising: administering a radiopharmaceutical composition comprising ¹⁷⁷Lu-PSMA &T and ascorbic acid in a solution having a pH of 3.5 to 4.5 more than 48 hours after formulation, wherein the solution has a radiochemical purity of more than 96% when administered.
Statement 69: The method of statement 68, further comprising imaging the patient using PSMA-PET to document and confirm the patient is mCRPC positive prior to administering the radiopharmaceutical composition.
Statement 70: The method of statement 68, wherein the patient has improved radiographic progression free survival (rPFS).
Statement 71: The method of statement 70, wherein the patient has an rPFS of about 6 months to 12 months.
Statement 72: The method of statement 68, wherein the patient has improved overall survival (OS).
Statement 73: The method of statement 72, wherein the patient has an OS of about 18 months to 25 months.
Statement 74: The method of statement 68, wherein the patient has improved second radiographic progression free survival (rPFS 2).
Statement 75: The method of statement 68, wherein the patient has improved progression free survival.
Statement 76: The method of statement 68, wherein the patient has improved second progression-free survival.
Statement 77: The method of statement 68, wherein the patient has an improved PSA₅₀ response rate.
Statement 78: The method of statement 68, wherein the patient has an improved time to first symptomatic skeletal event (SSE).
Statement 79: The method of statement 68, wherein the patient has an improved time to soft tissue progression (STP).
Statement 80: The method of statement 68, wherein the patient has an improved time to chemotherapy (TTC).
Statement 81: The method of statement 68, wherein the patient has improved results on a Quality of Life Questionnaire.
Statement 82: The method of statement 68, wherein the pH is 3.5 to 4.2.
Statement 83: The method of statement 68, wherein the composition comprises <6 μg of Lu-PSMA-I&T per mL of solution.
Statement 84: The method of statement 68, wherein the composition comprises about 13 μg to about 18 μg disodium EDTA per mL of solution, about 35 μL to about 40 μL ethanol per mL of solution, and about 31 mg/ml ascorbic acid.
Statement 85: The method of statement 68, wherein the composition comprises a radioactivity of about 0.5 GBq or about 13.5 mCi per mL of solution.
Statement 86: The method of statement 68, wherein the solution has a radiochemical purity of at least 98% at 44 hours after formulation, at least 97% at 69 hours after formulation, and/or at least 97% at 93 hours after formulation.
Statement 87: The method of statement 68, wherein upon administration of the composition to a patient, the patient maintains low levels of hematotoxic and nephrotoxic toxicity, and wherein the prostate-specific antigen decline is more than about 50%.
Statement 88: The radiopharmaceutical composition of statement 68, wherein the composition has a radioactivity of 1,270 MBq/ml to about 1,311 MBq/ml.
Statement 89: A radiopharmaceutical composition comprising ¹⁷⁷Lu-PSMA I&T, about 31 to about 42.5 mg/ml ascorbic acid, about 8 μg/ml to about 21 μg/ml disodium EDTA, and about 35 μl/ml to about 75 μl/ml ethanol in a solution having a pH of 3.5 to 4.5, wherein the solution has a radiochemical purity of more than 95% when administered.
Statement 90: The radiopharmaceutical composition of statement 89, wherein the composition has a high radioactivity of about 1,278 MBq/ml to about 1,311 MBq/ml.
Statement 91: The radiopharmaceutical composition of statement 89, wherein the composition has a low radioactivity of about 579 MBq/ml to about 626 MBq/ml.
Statement 92: The radiopharmaceutical composition of statement 89, wherein Lu-PSMA I&T is present in the composition in a concentration of less than about 12 μg/ml.
Statement 93: The radiopharmaceutical composition of statement 89, wherein the composition has an amount of colloidal ¹⁷⁷Lu of less than about 5% of radioactivity.
Statement 94: The radiopharmaceutical composition of statement 89, wherein the composition has less than about 17.5 EU/ml bacterial endotoxins.
Statement 95: The radiopharmaceutical composition of statement 89, wherein the composition has a shelf life of more than 24 hours after formulation.
Statement 96: The radiopharmaceutical composition of statement 95, wherein the composition has a shelf life of more than 48 hours after formulation.
Statement 97: The radiopharmaceutical composition of statement 96, wherein the composition has a shelf life of more than 72 hours after formulation.
Statement 98: A radiopharmaceutical composition comprising a ¹⁷⁷Lu-PSMA I&T solution for injection.
Statement 99: The radiopharmaceutical composition of statement 98, wherein the ¹⁷⁷Lu-PSMA I&T solution for injection comprises ¹⁷⁷Lu-PSMA I&T, ascorbic acid, and ethanol, wherein the ¹⁷⁷Lu-PSMA I&T is in sufficient amounts of radioactivity for intended use, wherein the total amount of ascorbic acid in the solution is about 210-700 mg and the total amount of ethanol in the solution is about 274-706 mg, wherein pH of the solution is about 5 or below, wherein upon administration of the composition to a subject, the subject maintains low levels of hematotoxic and nephrotoxic toxicity; and wherein the prostate-specific antigen decline is more than about 50%.
Statement 100: The radiopharmaceutical composition of statement 98, wherein the ¹⁷⁷Lu-PSMA I&T solution for injection comprises ¹⁷⁷Lu-PSMA-I&T in an amount from about 5 μg/ml to about 15 μg/ml, ascorbic acid in a concentration from about 10 mg/ml to about 50 mg/ml, and ethanol in a concentration of about 1% (v/v) to about 10% (v/v), wherein pH of the solution is between about 3 and about 5, and wherein upon administration of the composition to a subject, the subject maintains low levels of hematotoxic and nephrotoxic toxicity.
Statement 101: The radiopharmaceutical composition of statement 100, wherein the composition has a radioactivity of less than about 300 mCi.
Statement 102: The radiopharmaceutical composition of statement 100, wherein the composition comprises ethanol in an amount of about 1% (v/v), about 2% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v).
Statement 103: The radiopharmaceutical composition of statement 100, wherein the composition comprises ascorbic acid in an concentration of about 10 mg/ml, about 15 mg/ml, about 20 mg/ml, about 25 mg/ml, about 21 mg/ml, about 25 mg/ml, about 30 mg/ml, about 31 mg/ml, about 35 mg/ml, about 40 mg/ml, about 42.5 mg/ml, about 45 mg/ml, about 50 mg/ml, about 55 mg/ml, about 60 mg/ml, about 65 mg/ml, about 70 mg/ml, about 75 mg/ml, or about 80 mg/ml.
Statement 104: The radiopharmaceutical composition of statement 100, wherein the composition has a radioactive content from about 70% to about 130%.
Statement 105: The radiopharmaceutical composition of statement 104, wherein the composition provides a mean whole-body effective dose of about 23±20 Gy (3.3 Gy/GBq) after administration in a subject in need thereof.
Statement 106: The radiopharmaceutical composition of statement 100, wherein the composition is sterile.
Statement 107: The radiopharmaceutical composition of statement 100, wherein the composition has a volume from about 1 ml to about 50 ml.
Statement 108: The radiopharmaceutical composition of statement 100, wherein the composition has a radiochemical purity of at least 97% as measured by HPLC at 0 hour post EOS.
Statement 109: The radiopharmaceutical composition of statement 98, wherein the ¹⁷⁷Lu-PSMA I&T solution for injection comprises ¹⁷⁷Lu-PSMA-I&T in an amount from about 5 μg/ml to about 15 μg/ml, ascorbic acid in a concentration from about 10 mg/ml to about 50 mg/ml, ethanol in a concentration of about 1% (v/v) to about 10% (v/v), and a chelating agent in an amount from about 0.001% to about 0.15% (w/w) of the total weight of the radiopharmaceutical composition, wherein pH of the solution is between about 3 and about 5, and wherein upon administration of the composition to a subject, the subject maintains low levels of hematotoxic and nephrotoxic toxicity.
Statement 110: The radiopharmaceutical composition of statement 109, wherein the composition has a radioactivity of less than about 500 mCi.
Statement 111: The radiopharmaceutical composition of statement 109, wherein the composition comprises ethanol in an amount of about 1% (v/v), about 2% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v).
Statement 112: The radiopharmaceutical composition of statement 109, wherein the composition comprises ascorbic acid in an concentration of about 10 mg/ml, about 15 mg/ml, about 20 mg/ml, about 25 mg/ml, about 21 mg/ml, about 25 mg/ml, about 30 mg/ml, about 31 mg/ml, about 35 mg/ml, about 40 mg/ml, about 42.5 mg/ml, about 45 mg/ml, about 50 mg/ml, about 55 mg/ml, about 60 mg/ml, about 65 mg/ml, about 70 mg/ml, about 75 mg/ml, or about 80 mg/ml.
Statement 113: The radiopharmaceutical composition of statement 109, wherein the chelating agent is present in an amount of about 0.001%, about 0.002%, about 0.003%, about 0.004%, about 0.005%, about 0.01%, about 0.02%, about 0.03%, about 0.04%, about 0.05%, about 0.06%, about 0.07%, about 0.08%, about 0.09%, about 0.10%, about 0.11%, about 0.12%, about 0.13%, about 0.14%, or about 0.15% (w/w) of the total weight of the radiopharmaceutical composition.
Statement 114: The radiopharmaceutical composition of statement 109, wherein the composition has a radioactive content from about 70% to about 130%.
Statement 115: The radiopharmaceutical composition of statement 109, wherein the composition provides a mean whole-body effective dose of about 23±20 Gy (3.3 Gy/GBq) after administration in a subject in need thereof.
Statement 116: The radiopharmaceutical composition of statement 109, wherein the composition is sterile.
Statement 117: The radiopharmaceutical composition of statement 109, wherein the composition has a volume from about 1 ml to about 50 ml.
Statement 118: The radiopharmaceutical composition of statement 109, wherein the composition has a radiochemical purity of at least 97% as measured by HPLC at 0 hour post EOS.
Statement 119: A radiopharmaceutical kit comprising a vial containing at least a single dose of a ¹⁷⁷Lu-PSMA I&T solution for injection to a human patient in need thereof, wherein the injection comprises a 7.4 GBq±0.1 GBq dose.
Statement 120: The radiopharmaceutical kit of statement 119, wherein the vial is a sterile pyrogen free glass vial of Type 1 glass with a fluorocoated bromobutyl rubber septum.
Statement 121: The radiopharmaceutical kit of statement 120, wherein the septum is sealed with a crimped aluminum capsule.
Statement 122: The radiopharmaceutical kit of statement 119, further comprising a lead shielded transport container, wherein the glass vial is kept in a lead shielded container during transportation.
Statement 123: The radiopharmaceutical kit of statement 122, wherein the lead shielded transport container complies to type A requirements (IAEA standards).
Statement 124: The radiopharmaceutical kit of statement 119, wherein the vial contains multiple doses.
Statement 125: The radiopharmaceutical kit of statement 119, wherein the ¹⁷⁷Lu-PSMA &T solution has a volume from about 10 ml to about 20 ml, from about 20 ml to about 30 ml, from about 30 ml to about 40 ml, from about 40 ml to about 50 ml, from about 50 ml to about 60 ml, from about 60 ml to about 70 ml, from about 70 ml to about 80 ml, from about 80 ml to about 90 ml, or from about 90 ml to about 100 ml.
Statement 126: The radiopharmaceutical kit of statement 119, wherein the ¹⁷⁷Lu-PSMA &T solution has a strength of about 0.1 GBq/ml, about 0.2 GBq/ml, about 0.3 GBq/ml, about 0.4 GBq/ml, about 0.5 GBq/ml, about 0.6 GBq/ml, about 0.7 GBq/ml, about 0.8 GBq/ml, about 0.9 GBq/ml, about 1.0 GBq/ml, about 1.1 GBq/ml, about 1.2 GBq/ml, about 1.3 GBq/ml, about 1.4 GBq/ml, about 1.5 GBq/ml, about 1.6 GBq/ml, about 1.7 GBq/ml, about 1.8 GBq/ml, about 1.9 GBq/ml, or about 2.0 GBq/ml.
Statement 127: The radiopharmaceutical kit of statement 119, wherein the ¹⁷⁷Lu-PSMA &T solution is suitable for administration to a human patient in need thereof more than 72 hours after formulation, more than 96 hours after formulation, or more than 100 hours after formulation.
Statement 128: The radiopharmaceutical kit of statement 119, wherein the ¹⁷⁷Lu-PSMA &T solution has a pH of 3.5 to 4.5.
Statement 129: A method of diagnosing or treating a tumor of a patient in need thereof, the method comprising administering by injection a radiopharmaceutical composition comprising ¹⁷⁷Lu-PSMA &T and ascorbic acid at a pH of 3.5 to 4.5 in solution, wherein 20 hours after injection the radiopharmaceutical composition has an activity of at least 20% IA to 30% IA in the whole body.
Statement 130: The method of statement 129, wherein 40 hours after injection the radiopharmaceutical composition has an activity of at least 10% IA to 20% IA in the whole body.
Statement 131: The method of statement 129, wherein 60 hours after injection the radiopharmaceutical composition has an activity of at least 5% IA to 10% IA in the whole body.
Statement 132: A method of diagnosing a tumor of a patient in need thereof, the method comprising administering to the patient by injection a radiopharmaceutical composition comprising ¹⁷⁷Lu-PSMA &T and ascorbic acid at a pH of 3.5 to 4.5 in solution, wherein less than 20 hours after injection the radiopharmaceutical composition has an activity of at least 8% IA to 10% IA in the kidneys.
Statement 133: The method of statement 132, wherein 20 hours after injection the radiopharmaceutical composition has an activity of at least 3% IA to 8% IA in the kidneys.
Statement 134: The method of statement 132, wherein 40 hours after injection the radiopharmaceutical composition has an activity of at least 1% IA to 5% IA in the kidneys.
Statement 135: The method of statement 132, wherein 60 hours after injection the radiopharmaceutical composition has an activity of at least 1% IA to 5% IA in the kidneys.
Statement 136: A method of diagnosing a tumor in a patient in need thereof, the method comprising administering to the patient by injection a radiopharmaceutical composition comprising ¹⁷⁷Lu-PSMA &T and ascorbic acid at a pH of 3.5 to 4.5 in solution, wherein less than 20 hours after injection the radiopharmaceutical composition has an activity of at least 0.7% IA to 1% IA in the parotid glands.
Statement 137: The method of statement 136, wherein 20 hours after injection the radiopharmaceutical composition has an activity of at least 0.3% IA to 0.8% IA in the parotid glands.
Statement 138: The method of statement 136, wherein 40 hours after injection the radiopharmaceutical composition has an activity of at least 0.2% IA to 0.5% IA in the parotid glands.
Statement 139: The method of statement 136, wherein 60 hours after injection the radiopharmaceutical composition has an activity of at least 0.1% IA to 0.3% IA in the parotid glands.
Statement 140: A method of diagnosing a tumor in a patient in need thereof, the method comprising administering to the patient by injection a radiopharmaceutical composition comprising ¹⁷⁷Lu-PSMA &T and ascorbic acid at a pH of 3.5 to 4.5 in solution, wherein less than 20 hours after injection the radiopharmaceutical composition has an activity of at least 0.2% IA to 0.5% IA in lymph node lesions of the patient.
Statement 141: The method of statement 140, wherein 20 hours after injection the radiopharmaceutical composition has an activity of at least 0.1% IA to 0.3% IA in the lymph node lesions.
Statement 142: The method of statement 140, wherein 40 hours after injection the radiopharmaceutical composition has an activity of at least 0.08% IA to 0.2% IA in the lymph node lesions.
Statement 143: The method of statement 140, wherein 60 hours after injection the radiopharmaceutical composition has an activity of at least 0.05% IA to 0.1% IA in the lymph node lesions.
Statement 144: A method of diagnosing a tumor in a patient in need thereof, the method comprising administering to the patient by injection a radiopharmaceutical composition comprising ¹⁷⁷Lu-PSMA &T and ascorbic acid at a pH of 3.5 to 4.5 in solution, wherein less than 20 hours after injection the radiopharmaceutical composition has an activity of at least 0.1% IA to 0.4% IA in bone lesions of the patient.
Statement 145: The method of statement 144, wherein 20 hours after injection the radiopharmaceutical composition has an activity of at least 0.1% IA to 0.2% IA in the bone lesions.
Statement 145: The method of statement 144, wherein 40 hours after injection the radiopharmaceutical composition has an activity of at least 0.05% IA to 0.1% IA in the bone lesions.
Statement 146: The method of statement 144, wherein 60 hours after injection the radiopharmaceutical composition has an activity of at least 0.02% IA to 0.05% IA in the bone lesions.
Statement 147: A method of diagnosing a tumor in a patient in need thereof, the method comprising administering to the patient by injection a radiopharmaceutical composition comprising ¹⁷⁷Lu-PSMA &T and ascorbic acid at a pH of 3.5 to 4.5 in solution, wherein the radiopharmaceutical composition has an effective half-life of about 30 hours to 40 hours in the whole body of the patient.
Statement 148: The method of statement 147, wherein the effective half-life of the radiopharmaceutical composition is about 25 hours to 35 hours in the kidneys of the patient.
Statement 149: The method of statement 147, wherein the effective half-life of the radiopharmaceutical composition is about 20 hours to 30 hours in the parotid glands of the patient.
Statement 150: The method of statement 147, wherein the effective half-life of the radiopharmaceutical composition is about 45 hours to 55 hours in bone lesions of the patient.
Statement 151: The method of statement 147, wherein the effective half-life of the radiopharmaceutical composition is about 35 hours to 45 hours in lymph node lesions of the patient.
Statement 152: A method of diagnosing a tumor in a patient in need thereof, the method comprising administering to the patient by injection a radiopharmaceutical composition comprising ¹⁷⁷Lu-PSMA &T and ascorbic acid at a pH of 3.5 to 4.5 in solution, wherein a mean absorbed dose of the radiopharmaceutical is about 0.01 mGy/MBq to 0.5 mGy/MBq in the whole body of the patient.
Statement 153: The method of statement 152, wherein the mean absorbed dose of the radiopharmaceutical composition is about 0.5 mGy/MBq to 1.0 mGy/MBq in the kidneys of the patient.
Statement 154: The method of statement 152, wherein the mean absorbed dose of the radiopharmaceutical composition is about 1 mGy/MBq to 1.5 mGy/MBq in the parotid glands of the patient.
Statement 155: The method of statement 152, wherein the mean absorbed dose of the radiopharmaceutical composition is about 2.5 mGy/MBq to 3.5 mGy/MBq in bone lesions of the patient.
Statement 156: The method of statement 152, wherein the mean absorbed dose of the radiopharmaceutical composition is about 3.5 mGy/MBq to 4.5 mGy/MBq in lymph node lesions of the patient.
Statement 157: A radiopharmaceutical kit, comprising a vial containing at least a single dose of a ¹⁷⁷Lu-PSMA &T solution for injection to a human patient in need thereof, wherein the injection comprises a ≥7.1 GBq dose.

What is claimed is:

1. A radiopharmaceutical composition comprising ¹⁷⁷Lu-PSMA I&T and ascorbic acid at a pH of 3.5 to 4.5 in solution, wherein the composition is suitable for administration to a human patient in need thereof at least 90 hours after formulation, and wherein the composition has a radiochemical purity of 95% or greater at administration.

2. The radiopharmaceutical composition of claim 1, wherein the composition comprises <6 μg of Lu-PSMA I&T per mL of solution.

3. The radiopharmaceutical composition of claim 1, wherein the composition comprises about 13 μg to about 18 μg disodium EDTA per mL of solution.

4. The radiopharmaceutical composition of claim 1, wherein the composition comprises about 35 μL to about 40 μL ethanol per mL of solution.

5. The radiopharmaceutical composition of claim 1, wherein the composition does not include ethanol.

6. The radiopharmaceutical composition of claim 1, wherein the composition comprises a radioactivity of about 0.5 GBq or about 13.5 mCi per mL of solution.

7. The radiopharmaceutical composition of claim 1, wherein the composition comprises about 21 mg/ml to about 42.5 mg/ml ascorbic acid.

8. A radiopharmaceutical composition comprising ¹⁷⁷Lu, about 463 μg/mL PSMA I&T precursor, about 4 ml 0.4 M sodium acetate, about 1.6 mL 0.05 M hydrochloric acid, about 150 μl 20% L-ascorbic acid, and a specific activity ≤61 GBq in a total of 6 to 8 mL of solution.

9. The radiopharmaceutical composition of claim 8, wherein the ascorbic acid has a pH of 3.5 to 4.5.

10. The radiopharmaceutical composition of claim 8, wherein the solution has a pH of 3.5 to 4.5.

11. The radiopharmaceutical composition of claim 8, further comprising 1.5 ml ethanol-water in a 1:1 (v/v) ratio.

12.-34. (canceled)

35. A radiopharmaceutical composition comprising ¹⁷⁷Lu-PSMA I&T and ascorbic acid, wherein the composition is formulated as a solution for injection having a pH of 3.5 to 4.5 and the solution is suitable for administration more than 48 hours after formulation.

36. A radiopharmaceutical composition comprising:
¹⁷⁷Lu-PSMA I&T solution for injection comprising ¹⁷⁷Lu-PSMA I&T, ascorbic acid, and ethanol;
wherein the ¹⁷⁷Lu-PSMA I&T is in sufficient amounts of radioactivity for intended use;
wherein the total amount of ascorbic acid in the solution is about 210-700 mg and the total amount of ethanol in the solution is about 274-706 mg; wherein pH of the solution is about 5 or below;
wherein upon administration of the composition to a subject, the subject maintains low levels of hematotoxic and nephrotoxic toxicity; and
wherein the prostate-specific antigen decline is more than about 50%.

37. A radiopharmaceutical composition comprising ¹⁷⁷Lu-PSMA I&T solution for injection comprising:
(a)¹⁷⁷Lu-PSMA-I&T in an amount from about 5 μg/ml to about 15 μg/ml;
(b) ascorbic acid in a concentration from about 10 mg/ml to about 50 mg/ml;
(c) optionally ethanol in a concentration of about 1% (v/v) to about 10% (v/v); and
(d) optionally a chelating agent in an amount from about 0.001% to about 0.15% (w/w) of the total weight of the radiopharmaceutical composition;
wherein pH of the solution is between about 3 and about 5;
wherein the composition is sterile;
wherein the composition has a volume from about 1 ml to about 50 ml; and
wherein upon administration of the composition to a subject, the subject maintains low levels of hematotoxic and nephrotoxic toxicity.

38. The radiopharmaceutical composition of claim 37, wherein the composition has a radioactivity of less than about 300 mCi.

39. The radiopharmaceutical composition of claim 37, wherein the composition comprises ascorbic acid in an concentration of about 10 mg/ml, about 15 mg/ml, about 20 mg/ml, about 25 mg/ml, about 21 mg/ml, about 25 mg/ml, about 30 mg/ml, about 31 mg/ml, about 35 mg/ml, about 40 mg/ml, about 42.5 mg/ml, about 45 mg/ml, about 50 mg/ml, about 55 mg/ml, about 60 mg/ml, about 65 mg/ml, about 70 mg/ml, about 75 mg/ml, or about 80 mg/ml.

40. The radiopharmaceutical composition of claim 37, wherein the composition has a radioactive content from about 70% to about 130%.

41. The radiopharmaceutical composition of claim 40, wherein the composition provides a mean whole-body effective dose of about 23±20 Gy (3.3 Gy/GBq) after administration in a subject in need thereof.

42. (canceled)

43. (canceled)

44. The radiopharmaceutical composition of claim 37, wherein the composition has a radiochemical purity of at least 97% as measured by HPLC at 0 hour post EOS.

45. A radiopharmaceutical composition comprising ¹⁷⁷Lu-PSMA I&T solution for injection comprising:
(a) ¹⁷⁷Lu-PSMA I&T in an amount from about 5 μg/ml to about 15 μg/ml;
(b) ascorbic acid in a concentration from about 10 mg/ml to about 50 mg/ml;
(c) ethanol in a concentration of about 1% (v/v) to about 10% (v/v); and
(d) a chelating agent in an amount from about 0.001% to about 0.15% (w/w) of the total weight of the radiopharmaceutical composition;
wherein pH of the solution is between about 3 and about 5; and
wherein upon administration of the composition to a subject, the subject maintains low levels of hematotoxic and nephrotoxic toxicity.

46.-60. (canceled)
